(19) United States
(12) Patent Application Publication
(10) Pub. No.: US 2014/0287433 A1
(43) Pub. Date: Sep. 25, 2014

(54) MEANS AND METHODS FOR DETERMINING CLOSTRIDIAL NEUROTOXINS

(71) Applicants: Oliver Weingart, Zürich (CH); Marc Avondet, Uetendorf (CH); Martin Loessner, Ebmatingen (CH); Andreas Rummel, Hannover (DE); Frank Gessler, Gleichen (DE)

(72) Inventors: Oliver Weingart, Zürich (CH); Marc Avondet, Uetendorf (CH); Martin Loessner, Ebmatingen (CH); Andreas Rummel, Hannover (DE); Frank Gessler, Gleichen (DE)

(73) Assignees: ETH ZÜRICH, Zürich (CH); BUNDESAMT FÜR BEVÖLKERUNGSSCHUTZ UND SPORT IM EIDGENÖSSISCHEN DEPARTEMENT FÜR; VERTEIDIGUNG, BEVÖLKERUNGSSCHUTZ UND SPORT, Bern (CH); TOXOGEN GMBH, Hannover (DE); MIPROLAB GMBH, Göttingen (DE)

(21) Appl. No.: 14/233,740

(22) PCT Filed: Jul. 18, 2012
(86) PCT No.: PCT/EP2012/064068

§ 371 Date: Apr. 9, 2014

(30) Foreign Application Priority Data
Jul. 19, 2011 (EP) 1105915.1

(51) Int. Cl. G01N 33/573

(52) U.S. Cl. CPC G01N 33/573
USPC 435/7.4; 264/4.1; 264/4.3

(57) ABSTRACT

This invention relates to a method of determining presence, amount and/or activity of a clostridial neurotoxin in a sample, the method comprising or consisting of the following steps: (a) bringing said sample into contact with a liposome, said liposome comprising (aa) at least one receptor on its outer surface, said receptor being capable of binding said neurotoxin and comprising or consisting of (i) a glycolipid and (ii) a peptide or protein; and (ab) a substrate in its interior, said substrate (i) being cleavable by the peptidase comprised in said neurotoxin and (ii) generating a detectable signal upon cleavage, said detectable signal preferably being generated by (1) the donor of a FRET pair, said donor exhibiting increased fluorescence upon cleavage by said peptidase, (2) a luminescent compound formed upon said cleavage, or (3) an enzyme formed upon said cleavage; and (b) determining whether an increase in signal occurs as compared to the absence of said sample, wherein such increase is indicative of the presence of said neurotoxin and/or the degree of such increase is indicative of the amount and/or activity of said neurotoxin in said sample.

This invention relates to a method of determining presence, amount and/or activity of a clostridial neurotoxin in a sample, the method comprising or consisting of the following steps: (a) bringing said sample into contact with a liposome, said liposome comprising (aa) at least one receptor on its outer surface, said receptor being capable of binding said neurotoxin and comprising or consisting of (i) a glycolipid and (ii) a peptide or protein; and (ab) a substrate in its interior, said substrate (i) being cleavable by the peptidase comprised in said neurotoxin and (ii) generating a detectable signal upon cleavage, said detectable signal preferably being generated by (1) the donor of a FRET pair, said donor exhibiting increased fluorescence upon cleavage by said peptidase, (2) a luminescent compound formed upon said cleavage, or (3) an enzyme formed upon said cleavage; and (b) determining whether an increase in signal occurs as compared to the absence of said sample, wherein such increase is indicative of the presence of said neurotoxin and/or the degree of such increase is indicative of the amount and/or activity of said neurotoxin in said sample.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Clostridial neurotoxins include various serotypes of botulinum neurotoxin and tetanus neurotoxin.

Botulinum neurotoxin (BoNT) is the substance with the highest toxic activity known to man. Intoxication with BoNT causes botulism, a gradually increasing neuroparalytic disease, which can lead to death by respiratory arrest [1]. Although four of the serotypes (A, B, E, and F) are known to cause human botulism, the majority of human cases are due to serotypes A and B [2]. BoNT consists of two individual subunits, Heavy Chain (HC) and Light Chain (LC), which are connected via a disulphide bond.

In the body, BoNT specifically binds via the C-terminal part of its HC(H_{C}) to receptors on the motoneuron's membrane, typically SV2C and the trisialoganglioside GT1b in case of BoNT/A, and Synaptotagmin-II (SytII) and GT1b in case of BoNT/B [3,4]. The toxin is internalised into the motoneuron via receptor-mediated endocytosis [5]. Following proton influx, the pH in the endosome is typically lowered from pH 7.2 to pH 5.3, and the HC N-terminal part (H_{N}) refolds into a transmembrane channel crossing the endosomal membrane, allowing for translocation of the LC into the cytosol of the motoneuron [6]. There, depending on the toxin's serotype, the LC specifically cleaves distinct SNARE-proteins (soluble N-ethylmaleimide-sensitive fusion protein attachment receptor). For example, the LC of BoNT/A specifically cleaves SNAP-25 (synaptosome associated protein of 25 kDa), and the LC of BoNT/B specifically cleaves Synaptobrevin/VAMP-2 (vesicle associated membrane protein) [7-9]. If one or more SNARE proteins are cleaved, then the SNARE complex cannot assemble anymore, release of neurotransmitter is inhibited, and hence, the following muscle cell is paralysed. The amount of toxin needed to cause severe symptoms of botulism and even death is extremely low. From studies in mice and monkeys it was calculated that already 1 ng per kg body weight via the intravenous route might be lethal in humans [10-12].

In spite of its extreme toxicity, the neurotoxin is used in modern biomedical applications [13-15]. If applied in minute doses, the toxin exerts a locally constrained paralysing effect, which is used for the treatment of a large variety of diseases, such as strabismus, hyperhidrosis, or chronic pain. Moreover, treatment of wrinkles, frown lines, or facial asymmetries and other applications of aesthetical surgery, present a large application area [16]. However, to guarantee patients' safety, it is crucial that the toxic activity in all batches of pharmaceutical and cosmetic BoNT preparations is consistent. Accordingly, strict controls apply, where potency in each batch is measured with the mouse LD50 test. In this test, mice are injected intraperitoneally with defined volumes of different dilutions of BoNT containing preparations and observed for typical botulism symptoms for up to 96 hours. The injected amount where 50% of mice die is considered the LD50, which is defined as 1 Unit of BoNT [17]. Prior to their death by respiratory arrest this test leaves the mice with severe suffering. Due to the enormous demand for BoNT containing pharmaceutical products, it is estimated that in Europe and USA, each year, more than 600,000 mice have to die for BoNT LD50 potency testing performed by pharmaceutical companies [18,19]. Despite intensified efforts, the mouse LD50 test is still the only detection method officially validated and accepted for testing the toxic activity of BoNT in pharmaceutical products [20]. This is because the test is able to assess the complex mode of action of BoNT in the body.

If an alternative assay aims to replace the mouse LD50 test, it would have to assess the following key actions: (1) Binding of the toxin to specific receptors on the membrane of motoneurons, (2) translocation of the LC from the endosome into the cell's cytosol, and (3) LC-mediated cleavage of distinct SNARE-protein(s). Although other methods have been published, none has so far been able to present an adequate alternative to the mouse LD50 test [23]. Most in vitro assays, for instance, are only able to detect one of the key actions of BoNT's biological activity, i.e. the LC cleavage activity or H_{C} binding to nerve cell receptors [24,25]. Although cell-based assays would in theory be ideal to test for all of the toxin's key actions, techniques for differentiation of neuronal stem cells into motoneurons are still in their infancy and primary nerve cell cultures provide only poor sensitivities, if compared with the mouse LD50 test [26-28]. Hence, detection of the key actions of BoNT toxic activity in the body requires a system that resembles that of living organisms but that offers at the same time properties such as reproducibility, easy readout and low maintenance costs.

The following Table 1 provides an overview of the currently available methods for determining botulinum neurotoxins.

TABLE 1

| | | | Determination of | | | | | Exemplary |
|---|---|---|---|---|---|---|---|---|
| Method | Mechanism | Duration | RB¹ | T² | C³ | Advantages | Disadvantages | Reference |
| Immunalogical | directed to | 5-6 h | − | − | − | low detection limit; | not directed to the | [21] |
| detection | epitopes of | | | | | easily reproducible; | mechanism of action of | |
| (ELISA, ECL, | light or heavy | | | | | high throughput | clostridial neurotoxins; | |
| LFA, immuno- | chain | | | | | | each serotype requires | |
| PCR, etc.) | | | | | | | a different specific | |
| | | | | | | | antibody; unknown | |
| | | | | | | | subtypes might escape | |
| | | | | | | | detection | |
| Receptor | detects binding | 2-8 h | + | − | − | low detection limit; | no determination of | [22] |
| binding | of the heavy | | | | | binding is generally | translocation and | |
| | chain to | | | | | independent of the | cleavage | |
| | receptor | | | | | serotype | | |
| Endo- | determines | 3-24 h | − | − | + | applicable to all | no determination of | [29] |
| peptidase | cleavage | | | | | serotypes to the | the activity of the | |
| test | activity of the | | | | | extent the | heavy chain; not very | |
| | light chain | | | | | substrate is known | sensitive | |
| Trans- | determines | n.a. | − | + | − | | so far only subject | [30] |
| location | formation of | | | | | | of basic research; | |
| test | the trans- | | | | | | no determination | |
| | membrane | | | | | | of the activity of | |
| | channel by | | | | | | the light chain | |
| | the heavy | | | | | | | |
| | chain (and | | | | | | | |
| | translocation | | | | | | | |
| | of the light | | | | | | | |
| | chain) | | | | | | | |
| Nerve cell | determining of | 24-72 h | + | + | + | embraces the complete | so far no functioning | [31] |
| cultures | toxicity via | | | | | mechanism of action of | model of the motoric | |
| | measurement of | | | | | botulinum neurotoxin; | endplate (which is the | |
| | the inhibition | | | | | depending on the cell | botulinum neurotoxin | |
| | of neuro- | | | | | line, detection limit | target cell) is | |
| | transmitter | | | | | is in the ng-pg/mL | available; labor and | |
| | release | | | | | range | cost intensive | |
| | | | | | | | (differentiation and | |
| | | | | | | | maintenance of cell | |
| | | | | | | | lines); difficult to | |
| | | | | | | | standardize; lack of | |
| | | | | | | | reproducibility; | |
| | | | | | | | low throughput | |
| Mouse | readout via | 1-3 h | + | + | + | determines complete | low throughput; | [32] |
| diaphragm | decrease of | | | | | mechanism of action of | labor intensive; | |
| preparation | diaphragm | | | | | botulinum neurotoxin | consumes animals | |
| | muscle | | | | | low detection limit | | |
| | contraction | | | | | | | |
| | upon contact | | | | | | | |
| | with botulinum | | | | | | | |
| | neurotoxin | | | | | | | |
| Modified | measuring of | days | + | + | + | determines complete | cost intensive | [33] |
| mouse | the time until | | | | | mechanism of action of | (keeping of | |
| tests | paralysis of | | | | | botulinum neurotoxin; | animals, assay); | |
| | certain muscle | | | | | low detection limit | time intensive | |
| | groups occurs | | | | | | | |
| Mouse | determining | up to | + | + | + | determines complete | disputed from | [34] |
| LD50- | via death of | 96 h | | | | mechanism of action of | an ethical point | |
| assay | the mouse | | | | | botulinum neurotoxin; | of view; time and | |
| | from botulism | | | | | approved for control of | cost intensive | |
| | | | | | | botulinum neurotoxin | | |
| | | | | | | preparations; low limit | | |
| | | | | | | of detection (20 pg/mL) | | |

¹Receptor binding
²Translocation
³Cleavage

A further example of a botulinum neurotoxin assay, which fails to determine the entire mechanism of action of the neurotoxin is described in US 2011/0033866. This document describes neurotoxin substrates suitable for a FRET assay. To the extent use is made of vesicles, the vesicles merely serve as a carrier for such substrate. The vesicle is not equipped with receptors and accordingly does not provide for determining receptor binding and/or translocation. Moreover, to the extent this document refers to assays making use of vesicles, FRET is not used as detection scheme.

The technical problem underlying the present invention can therefore be seen in the provision of alternative or improved means and methods for determining clostridial neurotoxins.

This problem is solved by the subject-matter of the claims. In particular, and in a first aspect, the present invention provides a method of determining presence, amount and/or activity of a clostridial neurotoxin in a sample, the method comprising or consisting of the following steps: (a) bringing said sample into contact with a liposome, said liposome comprising (aa) at least one receptor on its outer surface, said receptor being capable of binding said neurotoxin and comprising or consisting of (i) a glycolipid and (ii) a peptide or protein; and (ab) a substrate in its interior, said substrate (i) being cleavable by the peptidase comprised in said neurotoxin and (ii) generating a detectable signal upon cleavage, said detectable signal preferably being generated by (1) the donor of a FRET pair, said donor exhibiting increased fluorescence upon cleavage by said peptidase, (2) a luminescent compound formed upon said cleavage, or (3) an enzyme formed upon said cleavage; and (b) determining whether an increase in signal occurs as compared to the absence of said sample, wherein such increase is indicative of the presence of said neurotoxin and/or the degree of such increase is indicative of the amount and/or activity of said neurotoxin in said sample.

Said determining may be a merely qualitative determining, i.e. determining presence or absence of the clostridial neurotoxin. Alternatively or in addition, said determining may be quantitatively, i.e. the determining of amount and/or activity of a clostridial neurotoxin. Amount and activity to be determined may be relative or absolute amounts and/or activities. This will depend inter alia on how the method is calibrated and/or which controls and standards are used. Such specifics of the assay design are within the abilities of the skilled person. An exemplary illustration is comprised in the examples enclosed herewith.

Generally speaking, amount and activity will be proportional to each other across a significant range of values. Having said that, it is known in the art that a defined amount of a clostridial neurotoxin may exhibit different activity depending on presence or absence of accessory non-toxin proteins, also referred to as neurotoxin associated proteins (NAPs). The neurotoxin as such consists of light and heavy chain as discussed in the introductory section herein above. In case of serotype A of botulinum toxin, said neurotoxin has an approximate molecular weight of 150 kDa. Clostridium botulinum typically produces botulinum type A toxin complexes, wherein said complexes comprise said neurotoxin on the one hand and one or more non-toxin proteins on the other hand. As a consequence, botulinum type A toxin complexes are found which have molecular weights of about 900 kDa, 500 kDa or 300 kDa. Similar findings, although different in terms of molecular weight, apply to the other botulinum toxins, which is well-known in the art. Depending on the specific conditions chosen, the presence of non-toxin proteins may have a stabilizing effect, the consequence being that a given amount of neurotoxin, when provided in complex form, may have a different, for example higher activity as compared to the same amount of neurotoxin in the absence of non-toxin proteins. The stabilizing effect of non-toxin proteins as well as conditions where non-toxin proteins are dispensable are known to the skilled person. Furthermore, formulations of clostridial neurotoxins are known or at the skilled person's disposal, which, instead of said non-toxin proteins or in addition thereto, contain further stabilizing agents such as human serum albumin, sucrose and/or gelatine.

While said neurotoxin as comprised in said sample may be associated with NAPs, it is preferred that a sample comprising neurotoxin and being free of NAPs is subjected to the methods of the invention. Within said neurotoxin, presence of heavy chain and light chain is necessary, noting that the method probes for the concomitant occurrence of receptor binding, translocation and cleavage. It is understood that, as established in the art, the term “clostridial neurotoxin” refers to an entity comprising, preferably consisting of heavy and light chain (see also the introductory section above), either fused as single chain form or as di-chain form, and providing the activities required for said receptor binding, translocation and cleavage. As noted above, in case of BoNT/A, the neurotoxin has an approximate molecular weight of 150 kD. Sometimes the term “neurotoxic component” is used in the art to designate the entity consisting of heavy and light chain.

The term “amount” includes concentration, mass, weight, and amount of substance, and preferably is expressed in terms of a concentration. To the extent molecular weights are known, mass, weight and concentration on the one hand and amount of substance on the other hand can be interconverted.

Since the third step of the mechanism of action of clostridial neurotoxins is an enzymatic activity, more specifically a proteolytic activity (herein also referred to as peptidase activity), activity may be expressed in terms of the number of cleaved substrates per unit of time. In the methods according to the invention, the measured activity results from the efficacy of all three steps of the mechanism of action of the neurotoxin, i.e. receptor binding, translocation and substrate hydrolysis, herein also referred to as cleavage of the substrate. Receptor binding is governed inter alia by the affinity of the respective neurotoxin for the receptor according to the invention. Translocation, i.e. formation of a transmembrane channel and transport of the peptidase into the lumen of the liposome according to the invention, is triggered or enhanced by a pH shift (for details see below).

Since the same amounts of a given neurotoxin may exhibit different activities, such different activities may be expressed as specific activity, with specific activity typically being the activity per unit of mass or amount of substance. Typically the methods of the invention, when used for quantitative determination of neurotoxins, yield activities in the first step. If the specific activity of the sample is known or can be determined, activity may be converted into amounts. Alternatively, the amount, in particular in case of samples, which are concentrated solutions of neurotoxin, may be determined by means such as SDS-PAGE, photometry and ELISA. Upon determination of activity by the method of the invention and of the amount by such means, the specific activity may be calculated.

A further common measure of the activity of neurotoxins, in particular of botulinum neurotoxins, are units. A Unit is defined by reference to the above-mentioned mouse LD50 assay. A Unit is the median intraperitoneal lethal dose (LD50) in mice. In preferred embodiments of the method according to the first aspect of the invention, a reference sample may be used, said reference sample comprising a defined activity of clostridial neurotoxin, said defined activity preferably being expressed in terms of units. In such a case, the activity of sample can then be specified in terms of units as well.

Clostridial neurotoxins are neurotoxins produced by bacteria of the genus Clostridium. Toxin producing species include Clostridium botulinum, Clostridium butyricum, Clostridium baratii, Clostridium argentinense as well as Clostridium tetania. Clostridial neurotoxins are further detailed below. Clostridial neurotoxins are typically characterized by a similar mechanism of action, which, as detailed above, comprises binding to receptors on the target nerve cell, translocation through the membrane and cleavage of the target protein by the peptidase component of the neurotoxin. Receptors and peptidase substrate may be different for the various toxins. Generally, the receptor is an integral or peripheral membrane protein, which consists of or comprises one or more extracellular domains, thereby being accessible to the neurotoxin heavy chain for binding. As regards the substrates, these are typically SNARE proteins. Several serotypes of botulinum neurotoxin cleave the same SNARE protein, however, at different sites. In some instances, for a substrate to be cleaved, not only the cleavage site, but also one or more recognition sites as present in the natural substrate must be present in an artificial substrate which may be used in the methods of the present invention (for details see below). Despite these subtle differences, the clostridial neurotoxins share a common mechanism of action, wherein all three steps of said common mechanism of action are assayed by the methods according to the present invention.

A sample according to the invention is known to comprise or suspected of comprising at least one clostridial neurotoxin. Depending on its origin, said sample may comprise further constituents. Preferred samples are the subject of a preferred embodiment described in detail below. The sample to be subjected to the method of determining according to the present invention may not only comprise further constituents owing to its origin, but also as a consequence of the deliberate addition of such further constituents. Such deliberately added constituent may be a test compound or a plurality of test compounds, wherein it is to be determined whether such compounds are capable of modulating the activity of clostridial neurotoxins. On the other hand, the primary sample as it might be obtained from, for example, a patient, might be known to comprise or suspected of comprising neutralizing antibodies against one or more clostridial neurotoxins, but otherwise free of neurotoxins as such. In that case, it is envisaged to deliberately add one or more neurotoxins to such primary sample, thereby obtaining the sample to be subjected to the method according to the present invention. Such preferred aspects (screen; determining of antibodies) are the subject of preferred embodiments discussed in more detail below.

Said sample is to be brought into contact with a liposome, the liposome being further characterized by items (i) and (ii) as recited in step (a) of the method according to the first aspect of the invention. It is understood that said bringing into contact is to be effected under conditions, which allow binding of said neurotoxin, if present, to said receptor. The skilled person can readily establish such conditions, such conditions including, for example, buffered solutions. Further preferred/exemplary conditions are specified in the examples enclosed herewith. Said conditions, even though disclosed in conjunction with serotype B of botulinum toxin are not confined to their use in conjunction with said serotype, but instead may be used with any clostridial neurotoxin and across all embodiments comprised by the first aspect of the present invention. Preferably, said determining according to (b) is effected at least 1, 2, 3, 4, 5, 6, 7, 8, 9, 10, 12, 15, 20, 24, 36 or 48 hours after said bringing into contact according to (a). These preferred periods of time refer to the total incubation time.

As stated above, said liposome meets at least the requirements (i) and (ii). Such liposome may either be obtained from natural sources such as nerve tissue or nerve cell cultures, or, in the alternative, by preparation starting from defined constituents. Preference is given to the latter approach. A method of preparing a liposome is also the subject of the present invention; see the fourth aspect discussed further below. The use of defined constituents and defined procedures of manufacture of the liposomes provides for a reproducible method of determining according to the first aspect.

The liposome according to the invention, which is also subject of the third aspect described in more detail below, comprises a receptor and a substrate. A liposome is, as known in the art, a closed compartment formed by a lipid bilayer. Generally, liposomes are artificially prepared. It is understood that the term “liposome”, as used herein, extends to liposomes, which are prepared from naturally occurring cells or tissues.

The receptor is defined in both structural and functional terms. In structural terms, the receptor comprises or consists of a glycolipid on the one hand and a peptide or protein on the other hand. In functional terms, said receptor is required to be capable of binding said neurotoxin. As a consequence, the skilled person understands that said receptor is intended to mimic the neurotoxin receptor, as it is present on the surface of nerve cells. Accordingly, said glycolipid is preferably to be chosen from a glycolipid which naturally occurs in the membrane of nerve cells or a modified version thereof, said modified version being capable of acting as a constituent of said receptor being capable of binding said neurotoxin. Preferred glycolipids are described further below.

The second, proteinaceous component of said receptor is a protein naturally occurring in nerve cells and known to be involved in neurotoxin binding, or a fragment thereof (herein also referred to as peptide), or a modified version of said protein or fragment, wherein said fragment, i.e. said peptide as well as said modified version are capable of acting as a component of a receptor being capable of binding said neurotoxin. The skilled person, in view of the present knowledge on targets and mechanism of action of clostridial neurotoxins, and furthermore provided with the teaching of the present invention is in a position to devise receptors meeting both the structural and functional requirements according to item (i) of the first aspect. For example, various truncated, deleted and/or modified versions of, the respective naturally occurring protein may be prepared and tested for their capability of binding a clostridial neurotoxin or a heavy chain thereof. The testing of receptors is also described in Nishiki et al. [76], Nishiki et al. [48], Dong et al. [50] Dong et al. [77], Dong et al. [78], Rummel et al. [79] and Mahrhold et al. [32].

The substrate according to item (ii) may either comprise the cognate substrate of the respective clostridial neurotoxin to be assayed, or a subsequence thereof, such subsequence being cleavable by the peptidase comprised in the neurotoxin. Again, both substrates as well as several subsequences are known to the skilled person and further detailed herein below. Further suitable subsequences may be found by simple tests such truncation series of the natural substrate, the truncated substrates being subjected, for example, to the method according to the present invention, wherein the measured activity is compared to the activity, which is observed when the sequence of the wild type substrate is used.

The substrate further generates a detectable signal upon cleavage. In principle, any means may be used which generate a detectable signal upon cleavage. Various such means are known in the art. The signal may be emission of light, be it by fluorescence or luminescence. Light may be emitted by the substrate itself once it is cleaved, or may be generated as a result of a downstream event triggered by cleavage of the substrate. Preferred options are detailed below.

In one preferred embodiment, the substrate comprises a FRET pair, such FRET pair consisting of a fluorescence donor and a fluorescence acceptor (option (1)). The term “FRET” is well-known in the art and stands for fluorescence resonance energy transfer. Sometimes the “F” within “FRET” is also understood as an abbreviation of the name Förster, noting that a scientist named Förster described the physical laws governing the energy transfer between a fluorescence donor and a fluorescence acceptor. Said FRET pair is to be positioned such that fluorescence increases upon cleavage of the substrate by the peptidase. This implies positioning of donor and acceptor (i) on different sides of the substrate's cleavage site, and furthermore (ii) that donor and acceptor have an average spatial distance in the substrate that provides for sufficient quenching of the donor fluorescence by the acceptor in the uncleaved substrate. Distances between donor and acceptor for quenching to occur are typically in the order of up to 10 nM. Regardless thereof, suitable positions of donor and acceptor within a given substrate can be determined by the skilled person without further ado by simple tests.

In an alternative embodiment of the FRET pair of the invention, the acceptor does not (only) act as a quencher, but is capable of fluorescence emission, in particular when energy is transferred from a donor in sufficient spatial proximity. In that case, little or no donor emission, but significant acceptor emission will be measured in the uncleaved state of the substrate, while upon cleavage, the acceptor emission is decreased and the donor emission is increased. Suitable donor/acceptor pairs are known in the art and at the skilled person's disposal.

In a further preferred embodiment, a luminescent compound is formed upon cleavage (option (2)). Said luminescent compound may be, for example, oxyluciferin, which is formed from luciferin in the presence of the enzyme luciferase. Luciferase in turn may be provided in the form of two fragments of luciferase, said fragments being generated by said cleavage and, while being enzymatically inactive themselves, assemble into a functional enzyme having luciferase activity upon cleavage.

Generally speaking, it is also envisaged to use a substrate, which, upon cleavage, assembles to a yield a functional enzyme, wherein the activity of said enzyme is detected (option (3)). Such enzyme complementation system on the one hand and the generation of a luminescent compound on the other hand may characterize one and the same read-out scheme; see, for example, the luciferase system described above.

Preferred substrates are SNARE proteins comprising such FRET pair or otherwise modified to generate a signal upon cleavage, or fragments of SNARE proteins comprising FRET pairs or otherwise modified to generate a signal upon cleavage.

Step (b) provides for a comparison of signal generated by the liposome with and without sample, but otherwise preferably under the same conditions. To the extent substrates with FRET pairs are used, the following applies. For the purpose of determining any change in fluorescence, either the entire fluorescence spectrum may be recorded, or measurements may be effected at one or more specific wavelengths. A preferred wavelength is the wavelength of maximum fluorescence emission which is a characteristic known for most commercially available fluorophor or can be determined without further ado, for example by performing a spectral scan.

Said receptor is located on the outer surface of the liposome, and the substrate in the interior of the liposome, preferably only in the interior of the liposome. Preferred means of ensuring occurrence of the substrate only in the interior of the liposome include the following: (i) purification by means of size exclusion chromatography (as shown in FIG. 2 or alternatively in FPLC or HPLC format), (ii) purification by means of dialysis using a molecular weight cut-off which is above the molecular weight of the substrate, (iii) ultrafiltration with a molecular cut-off which is above the molecular weight of the substrate, and (iv) separation of liposomes by ultracentrifugation. These methods serve to separate the liposomes (with substrate molecules in their interior) from substrate molecules present in the mixture obtained from liposome preparation, the latter substrate molecules not being encapsulated in the liposomes.

While it is preferred that the substrate is soluble and dissolved in the aqueous medium in the lumen of the liposome, it is also envisaged to use membrane-bound substrates. In the latter case, alternative or additional means of ensuring that the substrate is pointing only inwards, i.e. into the lumen of the liposome, may be used. For example, substrate may be incorporated into the liposomes prior to incorporation of the receptor, at least of the peptide or protein component of the receptor. To the extent substrate molecules are pointing outwards, they may be digested by a suitable enzyme such as proteinase K or the light chain (LC) of the neurotoxin/serotype to be determined by the methods of the invention. In a subsequent step, the receptors are incorporated, thereby obtaining a liposome according to the present invention with membrane-bound substrate pointing only into the interior of the liposome. Said aqueous medium in the lumen of the liposome is such that cleavage of said substrate by said neurotoxin can take place. Such aqueous medium can be chosen by the skilled person without further ado. Preferred are the aqueous media disclosed further below in relation to the method of preparing a liposome according to the fourth aspect of this invention. A particularly preferred aqueous medium comprises or consists of HEPES buffer, preferably between 10 and 100 mM such as 20 mM, with a pH between 7 and 8, preferably 7.4, preferably supplemented with ZnSO₄, preferably at a concentration between 0.01 and 0.1 mM such as 0.05 mM, and furthermore preferably supplemented with TCEP, preferably at a concentration between 1 and 10 mM such as 2 mM.

In view of the above properties, said liposome mimics the characteristics of a nerve cell which are essential for the entire mechanism of action of a clostridial neurotoxin to be monitored, said mechanism of action comprising receptor binding, translocation and proteolytic cleavage of the target protein, see above. As is apparent from the review of the state of the art, the known neurotoxin assays either fail to test the entire mechanism of action or, to the extent they do, suffer from other deficiencies (see Table 1 herein above). The assay according to the first aspect of the present invention does not require mice (be it whole animals or diaphragm preparations obtained therefrom) nor are cultures of nerve cells needed. Due to the limited number of constituents and steps to be effected, the assay is robust, reproducible, amenable to standardization and capable of being effected in high throughput format. Sterile conditions are not required. Accordingly, a high throughput screen can be effected fast.

It is furthermore envisaged to use the assay according to the first aspect, wherein instead of said clostridial neurotoxins other bacterial toxins are to be determined. Such other toxins include the Bacillus anthracis toxin and diphtheria toxin. In an assay for Bacillus anthracis toxin, a commercially available substrate (MAPKKide) from List Biological Laboratories may be used. In case of diphtheria toxin, the enzymatic reaction occurring in the liposome is ADP-ribosylation of eukaryotic elongation factor 2 instead of proteolytic cleavage. This activity is described in the art and the assay according to the first aspect of the present invention may be readily adapted for the monitoring of said activity.

In a preferred embodiment, said clostridial neurotoxin is a botulinum neurotoxin, preferably type A, B, C1, D, E, F or G botulinum neurotoxin or tetanus neurotoxin. The various serotypes of botulinum toxin are known in the art and well characterized. Pharmaceutical or cosmetic compositions typically comprise botulinum neurotoxin type A (BoNT/A) or BoNT/B as active pharmaceutical ingredient.

In a further preferred embodiment, said sample is selected from a pharmaceutical composition, a diagnostic composition, a cosmetic composition, a clinical or patient sample, a food or feed sample, a beverage sample, a sample taken from a biotechnological process, a sample obtained from an animal and an environmental sample. As mentioned in the introductory section herein above, a variety of botulinum neurotoxin formulations are presently approved for medical and cosmetic uses. In the course of manufacture of these compositions, their testing is indispensable, in particular in view of the high toxicity of the active agent as well as its fragility and easy denaturation. Clinical samples, patient samples and samples obtained from an animal include samples taken from subjects or animals suspected to suffer from botulism. Alternatively, said samples may be from subjects suspected to contain neutralizing antibodies directed to one or more clostridial neurotoxins. Food, feed and beverage as well as environmental samples may be assayed for the presence of neurotoxin activity in order to determine whether any threat to the health of humans or animals is present. Samples taken from biotechnological processes include samples taken from biological processes for the manufacture of clostridial neurotoxins. For example, such a sample may be taken from a fermenter containing a bacterial culture expressing clostridial neurotoxins.

In a further preferred embodiment, said liposome comprises or consists of the following constituents: (a) (i) one or more liposome-forming lipids, preferably at least one phosphatidylcholine and cholesterol, said phosphatidylcholine preferably being selected from the group consisting of SPC, DOPC, and POPC; (ii) optionally tocopherol; (b) said at least one receptor, wherein said receptor preferably comprises or consists of (i) a glycolipid, preferably selected from the tri-sialo ganglioside GT1b, the di-sialo ganglioside GD1b and the di-sialo ganglioside GD1a; and (ii) a peptide or protein selected from (1) SV2C or a fragment thereof, wherein said fragment is capable of binding to said neurotoxin and preferably comprises or consists of the luminal domain 4 and at least one transmembrane domain; (2) synaptotagmin I or II or a fragment thereof, wherein said fragment is capable of binding to said neurotoxin and preferably comprises or consists of the N-terminal luminal domain and the transmembrane portion of synaptotagmin I or II; (3) SV2A or a fragment thereof, wherein said fragment is capable of binding to said neurotoxin and preferably comprises or consists of the luminal domain 4, preferably the C-terminal portion thereof; and (4) SV2B or a fragment thereof, wherein said fragment is capable of binding to said neurotoxin and preferably comprises or consists of the luminal domain 4, preferably the C-terminal portion thereof; (c) said substrate; and (d) an aqueous medium in the interior of said liposome.

In a further preferred embodiment, said one or more liposome formic lipids are selected from SPC, DOPC, POPC, Lecithin (egg yolk or soy bean), Asolectin (soy bean), posphatidylinositol, posphatidylserin, phosphatidic acid, phosphatidylethanolamine, phosphatidylglycerol, cardiolipin, DOPG (di-oleoyl-phosphatidylglycerol), DOPE (di-oleoyl-phosphatidylethanolamine) and POPG (palmitoyl oleoyl phosphatidylglycerol). Lecithin and Asolectin are lipid mixtures from natural sources. The remainder of lipids are preferably single molecular species. Moreover, also other amphiphilic molecules, either alone or in combination with any one of the above, can be used for the purpose of generating liposomes. The skilled person is aware of such further amphiphilic molecules. The lipids, lipid mixtures and amphiphilic molecules described above can be used in conjunction with any embodiment of the present invention.

Also provided is a liposome comprising or consisting of constituents (a), (b) and (c) as defined above as well as liposomes comprising or consisting of constituents (a) and (c); or constituents (a) and (b). In either case, said liposome may further comprise or further consist of constituent (d).

These embodiments further characterize the essential constituents of the liposome according to the present invention. In particular, constituent (a) provides the lipid bilayer forming compounds, constituent (b) provides the compounds forming the receptor, and constituent (c) is the substrate. As is apparent from the definition of constituents (a) and (b), each of said constituents may—either optionally or compulsory—comprise or consist of more than one compound.

In order to mimic closely the characteristics of eukaryotic cell membranes, at least one phosphatidylcholine as well as cholesterol are preferred. Among the preferred phosphatidylcholines, there is soy phosphatidylcholine (SPC), dioleylglycerol phosphocholine (DOPC) and palmitoyl oleoyl phosphocholine (POPC). Preference is given to DOPC and POPC. Preferably, also tocopherol, in particular D,L-alpha-tocopherol is present. In a further preferred embodiment, phosphatidic acid (PA) is furthermore present. Acidic lipids such as PA may have a beneficial effect on the activity of the peptidase of said neurotoxin. Further preferred compositions forming constituent (a) are apparent from the examples enclosed herewith. The liposome forming lipids as used in the examples as well as their relative amounts are generally applicable for the purposes of the present invention.

Receptors for clostridial neurotoxins as they naturally occur in nerve cells are typically made up by two components, namely a glycolipid and a protein. Accordingly, the receptor present on the liposomes according to the present invention, said receptor being capable of binding said neurotoxin, mimics such naturally occurring neurotoxin receptors. Preferred glycolipids are gangliosides and particularly preferred are the gangliosides according to (b)(i) of the present preferred embodiment. Gangliosides are glycosphingolipids. The above used designations for preferred gangliosides according to the present invention (i.e. GT1b, GD1b and GD1a) are established in the art; see, for example, Yowler and Schengrund [75]. The structures of said gangliosides are provided in the following: GD1b, Galβ3NAcGalβ4(NAcNeuα8NAcNeuα3)Galβ4GlcβCer; GD1a, NAcNeuα3Galβ3NAcGalβ4(N AcNeuα3)Galβ4GlcβCer; GT1b, NAcNeuα3Galβ3NAcGalβ4(NAcNeuα8NAcNeuα3)Galβ4Gl cβCer; wherein Cer is Ceramide; Gal is galactose; Glc is glucose; NAcGal is N-acetylgalactosamine and NAcNeu is sialic acid.

The proteinaceous component of the receptor is defined in part (b)(ii) of this embodiment. Either the full length naturally occurring proteins may be used or fragments thereof, the fragments being capable of binding to the respective neurotoxin. SV2A, SV2B and SV2C are forms of synaptic vesicle glycoprotein 2. Synaptic vesicle proteins are membrane trafficking proteins comprising 12 transmembrane segments in case of SV2 but only one in case of Syt-II and Syt-I.

Preferred peptides and proteins comprised in said receptor are provided in SEQ ID NOs: 1 to 12. More specifically, SEQ ID NO: 1 is a fusion protein of human SV2C, including the transmembrane domain, with glutathione S transferase (GST). The SV2C component of said fusion protein are residues 454 to 603 of human SV2C. SEQ ID NO: 2 consists of residues 454 to 603 of human SV2C. SEQ ID NO: 3 is a GST fusion protein with the luminal and transmembrane domain of rat Synaptotagmin I (residues 1 to 88). SEQ ID NO: 4 consists of residues 1 to 82 of rat Synaptotagmin I and accordingly comprises the luminal domain and the transmembrane domain. SEQ ID NO: 5 consists of residues 35 to 82 of rat Synaptotagmin I which is the minimal luminal domain including the transmembrane domain. SEQ ID NO: 6 is a fusion protein of GST with the luminal domain and the transmembrane domain of rat Synaptotagmin II (residues 1 to 90). SEQ ID NO: 7 consists of luminal and transmembrane domain of rat Synaptotagmin II (residues 1 to 90). SEQ ID NO: 8 consists of the minimal luminal domain and the transmembrane domain of rat Synaptotagmin II (residues 44 to 90). SEQ ID NO: 9 is a fusion protein of GST with the luminal domain 4 and the transmembrane domain of rat SV2A. SEQ ID NO: 10 consists of luminal domain 4 and transmembrane domain of rat SV2A (residues 469 to 619). SEQ ID NO: Ills a fusion protein of GST with the luminal domain 4 and the transmembrane domain of rat SV2B. SEQ ID NO: 12 consists of luminal domain 4 and transmembrane domain of rat SV2B (residues 413 to 560).

As stated above, said substrate is located in the interior of the liposome. Preferably, it is exclusively located in the interior of the liposome. Moreover, preference is given to a soluble substrate such that the substrate is located in the lumen of the liposome. Alternatively or in addition, it is envisaged that the substrate may comprise a transmembrane segment and/or a membrane anchor, such membrane anchor being provided, for example, by covalently attached hydrophobic molecules such as fatty acids. While generally preference is given to soluble substrates, it is noted that, in conjunction with serotype C of botulinum neurotoxin, preference is given to a membrane attached substrate, said substrate still being located inside the liposome.

In a further preferred embodiment said bringing into contact is effected at a pH between 6 and 8, preferably between 7 and 7.4, more preferably at about 7.2.

In a further preferred embodiment, after step (a) and prior to step (b), the pH is changed to a value between 4 and 6, preferably between 5 and 5.4, more preferably about 5.2. Further preferred pH values are 3.8, 3.9, 4.1, 4.2, 4.3, 4.4, 4.5, 4.6, 4.7, 4.8, 4.9, 5.1, 5.3, 5.5, 5.6, 5.7, 5.8 and 5.9. Preferably, said change of pH is effected at least 0.1, 0.2, 0.3, 0.4, 0.5, 0.6, 0.7, 0.8, 0.9, 1, 2, 3, 4, 5, 6, 7, 8, 9, 10, 12, 15, 20, 24, 36 or 48 hours after said bringing into contact. These periods of time specify the binding incubation time.

A pH-shift may be induced by addition of an acid, preferably a membrane impermeable acid (e.g. DMG=Dimethylglutaric acid, MES=Morpholinoethanesulfonic acid or others) to the mixture of liposomes and botulinum neurotoxin in buffer (e.g. HEPES buffer at pH 7.2). Another way to adjust the pH may be performed as follows: After incubation of botulinum neurotoxin and liposomes, preferably both with as little dilution as possible, preferably at 4° C. (on ice), preferably for 5-120 minutes, the mixture is transferred, for example into the cavities of black 96-well microplates (preferably pre-heated to 37° C.) and buffer, preferably HEPES buffer, with the appropriate pH (preferably between pH 4 to 6, preferably preheated to 37° C.) is added to give a desired pH as well as a specific concentration of liposomes and botulinum neurotoxin in the final reaction volume. Desired pH values are preferably selected from 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 and 6.0.

These preferred embodiments provide for a pH shift when performing the methods according to the present invention. While not strictly necessary, such shift in pH facilitates the formation of a membrane channel by a portion of the heavy chain of neurotoxin, said membrane channel providing for translocation of peptidase comprised by the light chain across the membrane.

If said bringing into contact is effected at a pH of 6, it is preferred that after step (a) and prior to step (b) the pH is changed to a value below 6.

In a further preferred embodiment, said determining according to (b) is effected at least 0.1, 0.2, 0.3, 0.4, 0.5, 0.6, 0.7, 0.8, 0.9, 1, 2, 3, 4, 5, 6, 7, 8, 9, 10, 12, 15, 20, 24, 36 or 48 hours after said change of pH. These periods of time specify the translocation and cleavage incubation time.

In a further preferred embodiment, said liposome has a mean diameter between 50 and 500 nm, preferably between 100 and 200 nm, most preferred about 150 nm. Any size within these intervals, such as 250, 300, 350, 400 and 450 nm as well as larger diameters such 600, 700, 800 and 900 nm or 1 μm are deliberately envisaged. As detailed in the examples enclosed herewith, liposomes may be prepared in such a manner that they exhibit a defined, preferably narrow size distribution. The means for ensuring such size distribution as defined in the examples are generally applicable to any liposome according to the present invention.

In a further preferred embodiment, said liposome comprises or consists of: (i) DOPC and/or POPC; (ii) cholesterol; (iii) GT1b, GD1b and/or GD1a; (iv) (1) SV2C or said fragment thereof; (2) synaptotagmin I or II or said fragment thereof; (3) SV2A or said fragment thereof; and/or (4) SV2B or said fragment thereof; (v) said substrate; (vi) an aqueous medium in the interior of said liposome; and (vii) optionally tocopherol.

The aqueous medium is preferably as defined herein above as well as further below.

In a second aspect, the present invention provides the use of a liposome as defined in any one of the preceding claims for determining presence, amount and/or activity of a clostridial neurotoxin.

The present invention furthermore relates in a third aspect to a liposome as defined in any one of the preceding claims. Such liposomes may either be obtained from nerve cells or nerve tissues via procedures known in the art (synaptosome preparations). Alternatively, the liposomes are prepared by the methods of the invention as described further below.

In a further preferred embodiment of the methods according to the first aspect, said sample is known to comprise or suspected of comprising neutralising antibodies against said neurotoxin, wherein said sample, prior to subjecting it to said method, is combined with a known amount or activity of said neurotoxin, and wherein a decreased amount or activity of said neurotoxin as determined by said method in comparison to a control sample is indicative of the presence of said neutralising antibodies, wherein said control sample comprises said known amount or activity of said neurotoxin but is free of said neutralising antibodies.

In the context of a running or intended pharmaceutical or cosmetic treatment with a botulinum neurotoxin, it may be of interest to determine whether the patient or subject has developed or is in the course of developing neutralizing antibodies against the neurotoxin. Such neutralizing antibodies may interfere with the effects triggered by the neurotoxin and may prevent any beneficial effect altogether. In such a case it might be considered, for example, to switch serotype. In order to properly decide when such measures should be considered, monitoring of presence and amount of neutralizing antibodies may be useful.

In the preferred embodiment of the method according to the invention designed for neutralizing antibody monitoring, use is made of a test sample and a control sample, wherein both samples comprise the same known amount or activity of neurotoxin.

In a further preferred embodiment, said sample comprises a test compound and a known amount or activity of said neurotoxin, wherein a decreased or increased amount or activity of said neurotoxin as determined by said method in comparison to a control sample is indicative of the test compound being an inhibitor or activator, respectively, of said neurotoxin, wherein said control sample comprises said known amount or activity of said neurotoxin but is free of said test compound.

This embodiment provides for a screening method for neurotoxin modulators, preferably inhibitors. Given the robustness and simplicity of the assay according to the present invention, it readily can be effected in high throughput format. In view of the FRET detection scheme, the readout may be effected in an automatic manner, for example by means of a CCD camera coupled to a data processing unit. Since liposomes may be kept in solution, the assay may be performed in wells of microtiter plates. Robotic systems for the handling of microtiter plates as known in the art may be employed.

In a fourth aspect, the present invention provides a method of preparing a liposome, said method comprising or consisting of the following steps: (a) dissolving (i) liposome-forming lipid(s), preferably at least one phosphatidylcholine and cholesterol, said phosphatidylcholine preferably being selected from the group consisting of SPC, DOPC, and POPC; (ii) GT1b; and optionally (iii) tocopherol in a suitable organic solvent; (b) evaporating said organic solvent; (c) resuspending the residue of step (b) in an aqueous medium, said aqueous medium comprising (ca) at least one receptor, said receptor being capable of binding a clostridial neurotoxin and comprising or consisting of a glycolipid and a peptide or protein, wherein preferably (i) said glycolipid is selected from the tri-sialo ganglioside GT1b, the di-sialo ganglioside GD1b and the di-sialo ganglioside GD1a; and (ii) said peptide or protein is selected from (1) SV2C or a fragment thereof, wherein said fragment is capable of binding to said neurotoxin and preferably comprises the luminal domain 4 and at least one transmembrane domain; (2) synaptotagmin I or II or a fragment thereof, wherein said fragment is capable of binding to said neurotoxin and preferably comprises the N-terminal extracellular domain and the transmembrane portion of synaptotagmin I or II; and (3) SV2A, SV2B or a fragment of SV2A or SV2B, wherein said fragment is capable of binding to said neurotoxin and preferably comprises or consists of the luminal domain 4; and (cb) a substrate (i) being cleavable by the peptidase comprised in said neurotoxin and (ii) generating a detectable signal upon cleavage, said detectable signal preferably being generated by (1) the donor of a FRET pair, said donor exhibiting increased fluorescence upon cleavage by said peptidase, (2) a luminescent compound formed upon said cleavage, or (3) an enzyme formed upon said cleavage; (d) extruding the suspension obtained in step (c) through a suitable membrane; and (e) optionally purifying the liposomes obtained in step (d), preferably by means of size exclusion chromatography.

Suitable organic solvents to be used in step (a) include polar solvents. Polar protic as well as polar aprotic solvents may be used, wherein preference is given to mixtures thereof. A preferred protic solvent is methanol and preferred aprotic solvents are dichloromethane and chloroform. Particularly preferred solvents are mixtures of methanol and dichloromethane and mixtures of methanol and chloroform, preferably 1:1 mixtures; see also Example 1 enclosed herewith.

The aqueous medium to be used in step (c) may comprise, in addition to the constituents recited in step (c) one or more buffers and/or salts. HEPES (4-(2-hydroxyethyl)-1-piperazine-ethanesulfonic acid) buffer, preferably at a concentration between 10 and 100 mM, and preferably at a pH between 7 and 8 may be used. Particularly preferred is 20 mM HEPES buffer at a pH of 7.4 and optionally 150 mM K-Glu or NaCl. A particularly preferred aqueous medium comprises or consists of HEPES buffer, preferably between 10 and 100 mM such as 20 mM, with a pH between 7 and 8, preferably 7.4, preferably supplemented with ZnSO₄, preferably at a concentration between 0.01 and 0.1 mM such as 0.05 mM, and furthermore preferably supplemented with TCEP, preferably at a concentration between 1 and 10 mM such as 2 mM. Alternatively, other known buffers with a pH between 7 and 8, preferably a pH of 7.4 may be used, for example phosphate buffered saline (for example 137 mM NaCl, 2.7 mM KCl, 8.1 mM Na₂HPO₄, 1.76 mM KH₂PO₄; also referred to as PBS) or Tris buffered saline (50 mM Tris.HCl and 150 mM NaCl; also referred to as TBS).

Preferably said aqueous medium is free of any detergent, in particular free of Tween-20.

Furthermore, it is preferred that said aqueous medium is free of dithiothreitol (DDT).

Suitable membranes to be used for preparing liposomes by means of extruding as recited in step (d) are known to the skilled person. Preferred membranes are polycarbonate membranes, which are available with various pore sizes.

Purification according to optional step (e) is a means of (i) removing components from the preparation obtained in step (d) which are not constituents of the formed liposomes, and (ii) obtaining a more uniform or narrow size distribution of liposomes. Other preferred means of purification are described further above.

As alternative to the method of preparing a liposome according to the fourth aspect, the present invention in addition provides the following methods.

In particular, provided is a method of preparing a liposome, said method comprising or consisting of the following steps: (a) dissolving a substrate in an aqueous medium; (b) adding liposome-forming lipids; (c) extruding the mixture obtained in (b) through a suitable membrane; (d) adding lactose or trehalose; (e) cooling the obtained mixture to a temperature between −50° C. and −100° C., preferably −80° C.; (f) subjecting the frozen mixture to freeze-drying; and (g) reconstituting the lyophilisate with aqueous medium.

Preferably, prior to said cooling according to step (e), said liposome solution is cooled in a first step to a temperature between 0 and 10° C., preferably 4° C., and in a second step to a temperature between 0 and −50° C., preferably −20° C. Said reconstituting according to (g) is preferably effected with 10 times concentrated aqueous medium, wherein after shaking, centrifuging and again shaking, distilled water is added until the desired 1-fold concentration is reached. Preferably, the receptor according to the invention is added concomitantly with said substrate.

In a further alternative, a method of preparing a liposome is provided, said method comprising or consisting of the following steps: (a) diluting a substrate in an aqueous medium; (b) adding liposome-forming lipids; (c) cooling the emulsion obtained in (b) to about 0° C.; (d) sonicating said solution. Preferably, the receptor according to the invention is added concomitantly with said substrate.

It is understood that in conjunction with these alternative methods of preparing a liposome according to the invention, the definition of terms as well as of preferred embodiments thereof is as specified herein in relation to the various aspects of the invention. This applies in particular to substrate, aqueous medium, liposome-forming lipids and membranes suitable for extruding. Further details of said alternative methods of preparing a liposome according to the invention can be taken from the examples. The methods described there are generally applicable for any substrate or receptor as defined herein. Moreover, the conditions used are applicable to all liposome-forming lipids disclosed herein.

Yet further means for preparing liposomes according to the invention are described in Estes [81] and Mertins [82] and references cited therein.

In a preferred embodiment of the methods, the use and the liposomes according to the present invention, said substrate consists of or comprises a compound of the following formula (I):

X-L-Y;

wherein L is a peptide or protein comprising or consisting of a sequence which is cleavable by said peptidase; “—” denotes a covalent bond, X-L-Y is preferably soluble in aqueous medium and/or free of any transmembrane domain or membrane anchor; and (a) X is moiety comprising or consisting of a FRET donor or acceptor; and Y is a moiety comprising or consisting of a FRET acceptor if X comprises or consists of a donor, or a FRET donor if X comprises or consists of an acceptor; or (b) X is a fragment of an enzyme, said enzyme preferably being luciferase; and Y is another fragment of said enzyme, said enzyme preferably being luciferase, wherein, upon cleavage of L by the peptidase of said neurotoxin, a functional enzyme comprising X and Y, said functional enzyme preferably having luciferase activity, is formed.

It is understood that prior to cleavage none of X and Y according to (b), be it alone or in combination, is enzymatically active.

This embodiment defines the above-mentioned substrate of the peptidase comprised in the neurotoxin more specifically in structural terms.

Noting that L is a peptide or protein, in one embodiment moieties X and Y are attached to the N- and C-terminus of said peptide or protein L, respectively. However, there is no strict necessity for such placement. With regard to option (a), it is sufficient that donor and acceptor are placed on different sides of the cleavage site. Considering that the cleavage site defines an N-terminal and a C-terminal portion of the peptide or protein, said N- and C-terminal portions being connected by the scissile bond, donor and acceptor may be placed anywhere in said N- and C-terminal portion, respectively, provided that they are in sufficient spatial proximity for fluorescence resonance transfer to occur in the uncleaved form of the substrate.

X-L-Y is preferably soluble in aqueous medium. Also, it is preferred that X-L-Y is free of any transmembrane domain or membrane anchor, such membrane anchor being, for example, a hydrophobic covalently attached moiety (for example palmitoyl side chains as present in naturally occurring SNAP-25).

In a preferred embodiment of the method, the use or the liposome of the present invention, L comprises or consists of (i) SNAP-25 or a fragment thereof, said fragment being cleavable by the peptidase comprised in said neurotoxin and preferably being selected from a sequence comprising or consisting of residues 93 to 206, 146 to 203, or 156 to 184 of SNAP-25; (ii) VAMP-2, VAMP-1, VAMP-3 or a fragment thereof, said fragment being cleavable by the peptidase comprised in said neurotoxin and preferably being selected from a sequence comprising or consisting of residues 30 to 62, 30 to 86, 38 to 62, 47 to 96, or 62 to 86 of VAMP-2; and/or (iii) Syntaxin-1, -2, -3 or a fragment thereof, said fragment being cleavable by the peptidase comprised in said neurotoxin and preferably being a sequence comprising or consisting of residues 196 to 259 of Syntaxin.

SEQ ID NOs: 13 to 21 provide sequences of preferred L moieties according to the present invention. In particular, SEQ ID NO: 13 is the sequence of residues 1 to 206 of rat SNAP-25. SEQ ID NO: 14 is a subsequence of SEQ ID NO: 13 consisting of residues 140 to 206 of SNAP-25. SEQ ID NO: 15 is the sequence of human VAMP-2/Synaptobrevin II. SEQ ID NO: 16 is the sequence of rat VAMP-2 Synaptobrevin II. SEQ ID NO: 17 is the sequence of rat VAMP1. SEQ ID NO: 18 is the sequence of rat VAMP-3. SEQ ID NO: 19 is the sequence of human Syntaxin-1. SEQ ID NO: 20 is the sequence of rat Syntaxin-1. SEQ ID NO: 21 is the sequence of mouse Syntaxin-1.

As will be discussed in more detail below, subsequences of the natural substrates are also substrates of the respective clostridial neurotoxins. The subsequence preferences may vary between serotypes or between botulinum neurotoxins and tetanus toxin.

The skilled person can determine by using simple tests whether a given fragment of any one of SNAP-25, VAMP-2, VAMP-1, VAMP-3 and Syntaxin qualifies as a substrate.

In further preferred embodiments, said substrate is selected from SNAPtide, SNAP Etide, VAMPtide, and SYNTAXide. This embodiment refers to commercially available botulinum neurotoxin substrates. In particular, SNAPtide as described in U.S. Pat. No. 6,504,006 is a SNAP-derived BoNT/A substrate, SNAP Etide is a BoNT/E substrate, VAMPtide is a VAMP-derived BoNT/B substrate and SYNTAXide is a Syntaxin-derived BoNT/C1 substrate. These substrates are available from List Biological Labs Inc.

In a further preferred embodiment of the methods, use or liposome of the present invention, (a) said neurotoxin is botulinum neurotoxin type A; said receptor comprises or consists of (i) GT1b and (ii) SV2C or said fragment thereof; and L comprises or consists of SNAP-25 or said fragment thereof, said fragment preferably consisting of residues 146 to 203 of SNAP-25; (b) said neurotoxin is botulinum neurotoxin type B; said receptor comprises or consists of (i) GT1b and (ii) synaptotagmin II or I or said fragment thereof; and L comprises or consists of VAMP-2, VAMP, VAMP-3 or said fragment of any of these, said fragment preferably consisting of residues 62 to 86 of VAMP-2; (c) said neurotoxin is botulinum neurotoxin type C1; said receptor comprises or consists of GT1b; and L comprises or consists of SNAP-25, said fragment thereof, said fragment thereof preferably consisting of residues 93 to 206 of SNAP-25, Syntaxin-1, or said fragment thereof, said fragment preferably consisting of residues 196 to 259 of Syntaxin-1; (d) said neurotoxin is botulinum neurotoxin type D; said receptor comprises or consists of (i) GT1b and (ii) SV2A, SV2B, SV2C or the fragment of any of these as defined above; and L comprises or consists of VAMP-2, VAMP, VAMP-3 or said fragment of any of these, said fragment preferably consisting of residues 38 to 62 of VAMP-2; (e) said neurotoxin is botulinum neurotoxin type E; said receptor comprises or consists of (i) GT1b and (ii) SV2A, SV2B, or the fragment of any of these as defined above; and L comprises or consists of SNAP-25 or said fragment thereof, said fragment preferably consisting of residues 156 to 184 of SNAP-25; (f) said neurotoxin is botulinum neurotoxin type F; said receptor comprises or consists of (i) GT1b and (ii) SV2A, SV2B, SV2C, or the fragment of any of these as defined above, said fragment of SV2A and SV2B preferably comprising or consisting of the three luminal domains of SV2A and SV2B, respectively; and L comprises or consists of VAMP-2, VAMP, VAMP-3 or said fragment of any of these, said fragment preferably consisting of residues 30 to 62 of VAMP-2; (g) said neurotoxin is botulinum neurotoxin type G; said receptor comprises or consists of (i) GT1b and (ii) synaptotagmin I or II or said fragment thereof; and L comprises or consists of VAMP-2, VAMP, VAMP-3 or said fragment of any of these, said fragment preferably consisting of residues 47 to 96 of VAMP-2 and/or (h) said neurotoxin is tetanus toxin; said receptor comprises or consists of (i) GT1b and (ii) SV2A, SV2B, SV2C, the fragment of any of these as defined above, or a GPI-anchored glycoprotein like Thy-1; and L comprises or consists of VAMP-2, VAMP, VAMP-3 or said fragment of any of these, said fragment preferably consisting of residues 30 to 86 of VAMP-2.

VAMP-2 is also known as synaptobrevin 2. VAMP is also known as synaptobrevin 1. VAMP-3 is also referred to as cellubrevin.

Several neurotoxins are capable of recognizing different receptors and/or cleaving different substrates. For example, both BoNT/B and BoNT/G bind to synaptotagmins I and II, wherein the affinities for binding are generally as follows: BoNT/B-synaptotagmin II>>BoNT/G-synaptotagmin I>BoNT/G-synaptotagmin II>BoNT/B-synaptotagmin I.

The recited substrates, to the extent they are fragments defined in terms of specific residue ranges, are generally susceptible to cleavage by the neurotoxin peptidase to the same extent as the protein they originate from.

This embodiment provides preferred neurotoxin/receptor/substrate combinations, wherein the substrate is defined in terms of L as recited in herein above. Accordingly, it is understood that the preferred fragments are to be modified by introduction of fluorophor A and B in order to obtain substrates according to the present invention.

In further preferred embodiments, X is o-aminobenzoyl (o-Abz) or fluorescein isothiocyanate (FITC) and/or Y is 2,4-dinitrophenyl (DNP) or DABCYL. In particular, it is envisaged to use o-Abz in conjunction with DNP and FITC in conjunction with DABCYL. In addition, a large variety of FRET pairs, available from several manufacturers, is at the skilled person's disposal.

In a fifth aspect, the present invention provides a kit comprising or consisting of (a) at least one receptor, said receptor being capable of binding a clostridial neurotoxin and comprising (1) a glycolipid and (2) a peptide or protein; (b) a substrate which (1) is cleavable by the peptidase comprised in said neurotoxin and (2) comprises a FRET pair, the donor of said FRET pair exhibiting increased fluorescence upon cleavage by said peptidase; and (c) optionally one or more liposome-forming lipids.

In a preferred embodiment, the kit according to the present invention further comprises or further consist of one or more of the following (a) a manual containing instructions for performing the method of determining according to the invention; and/or (b) a manual containing instructions for performing the method of a liposome according to the invention.

In a further preferred embodiment, the constituents (a), (b) and (c) of said kit are as defined in conjunction with any of the other aspects of the present invention as disclosed herein above.

The figures show:

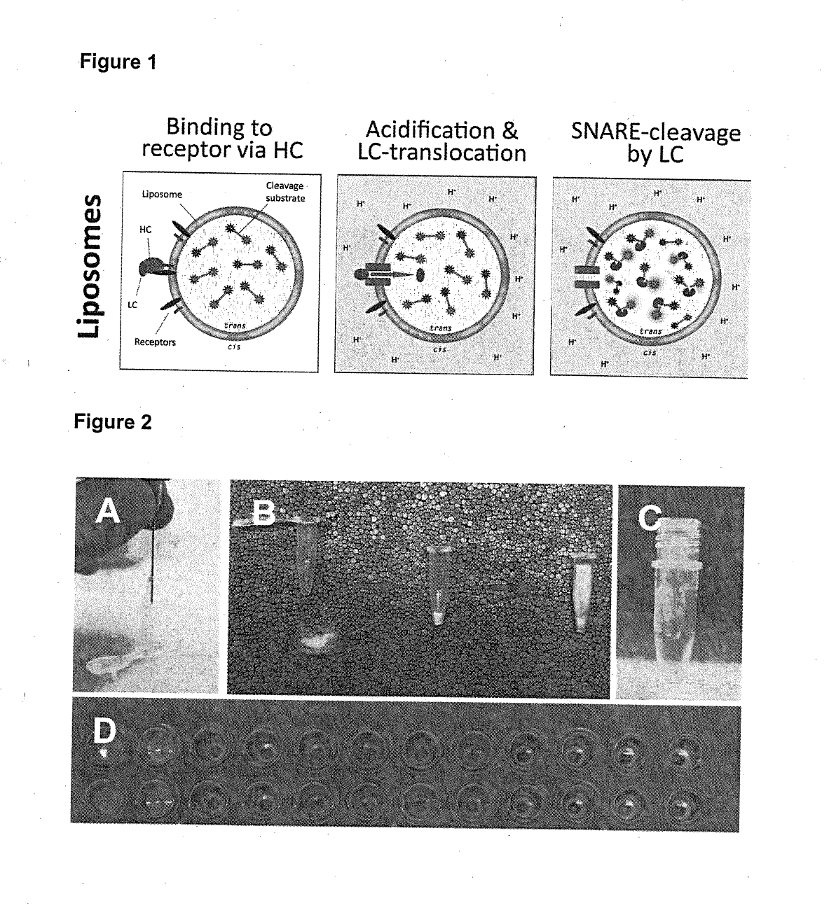

FIG. 1: Illustration of the method according to the first aspect of the invention. Binding: BoNT binds via its HC (oval) to nerve cell receptors (short and long bars) immobilized in the liposome membrane; translocation: conformational change of BoNT HC and insertion into a transmembrane channel upon pH-shift and translocation of BoNT LC (dark oval) into the liposome lumen; cleavage: enzymatic cleavage of the peptide reporter (acceptor fluorophor dark grey, donor fluorophor light grey) in the liposome lumen by the BoNT LC (dark oval with triangle opening). H⁺ represents free protons present upon acidification of the milieu surrounding the liposomes.

FIG. 2: Assembly of SEC-columns and illustration of collected fractions. (A) Puncturing of the 0.5 mL-Eppendorf tube (i.e. the later SEC column). (B) Insertion of the glass fibres as filter and filling with supporting material for the SEC chromatography medium (i.e. Sepharose 4B). (C) Fully assembled SEC-column with reservoir (1.5 mL screw-cap tube). (D) Separation shown for two samples of liposomes+100 μM calcein. Liposomes+encapsulated calcein are mainly found in the first two fractions, while calcein alone elutes in fractions four to nine.

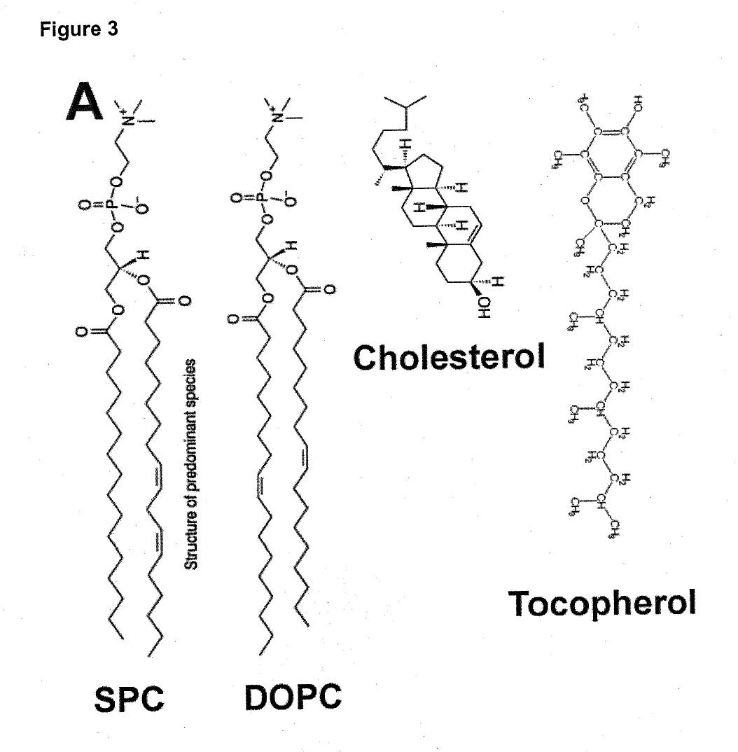

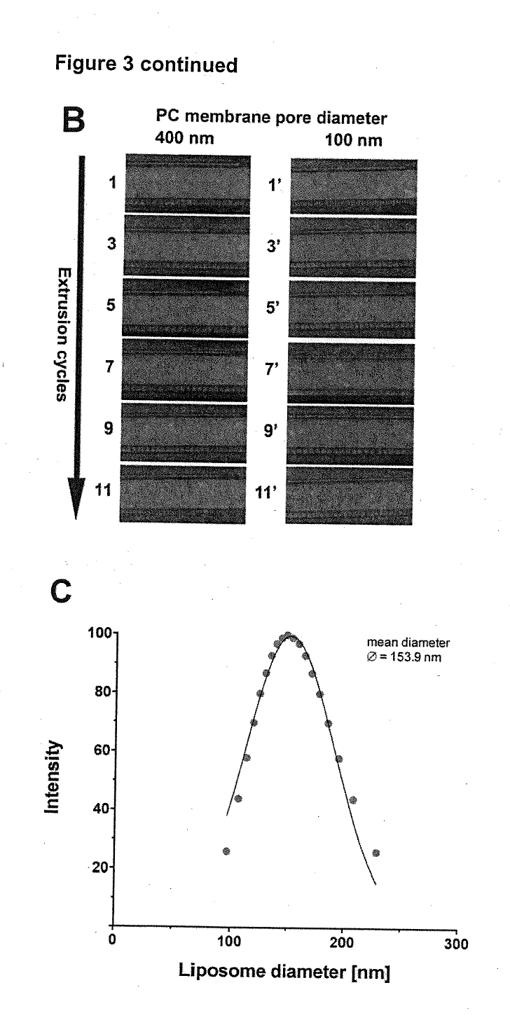

FIG. 3: Structure of lipids used for liposome production and extrusion of liposome emulsions. (A) Molecule structure of SPC, DOPC, cholesterol, and D,L-alpha-tocopherol. (B) Liposome suspension after consecutive extrusion through PC membranes with different pore sizes; numbers represent the respective extrusion cycles. (C) Distribution of liposome sizes after complete extrusion (filled circles), measured by DLS, resemble a Gaussian distribution (black line). Liposome suspension consisted of 20 mM SPC/Cholesterol/D,L-alpha-tocopherol (69.9:29.9:0.2).

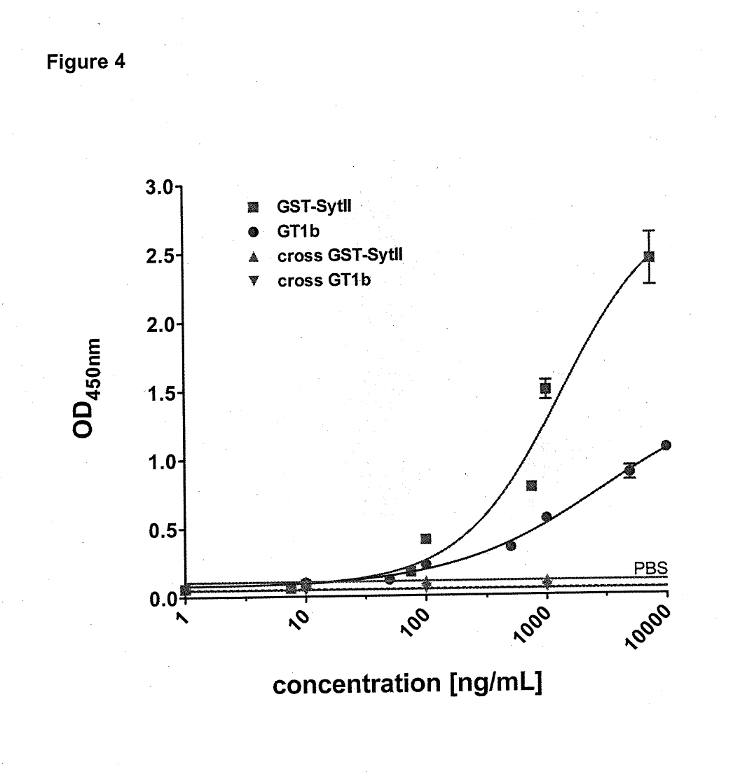

FIG. 4: Immunological detection of GT1b and GST-Synaptotagmin II (SytII) fusion protein. Sensitivity of ELISA immunoassay for detection of GST-SytII (squares) and GT1b (circles). Crossreactivity of the antibody pairing was tested by using GT1b-antibody for detection of GST-SytII (triangles), and GST-antibody for detection of GT1b (inverted triangles). Results for PBS (negative control) are shown as dotted black line. Error bars represent the respective standard deviation.

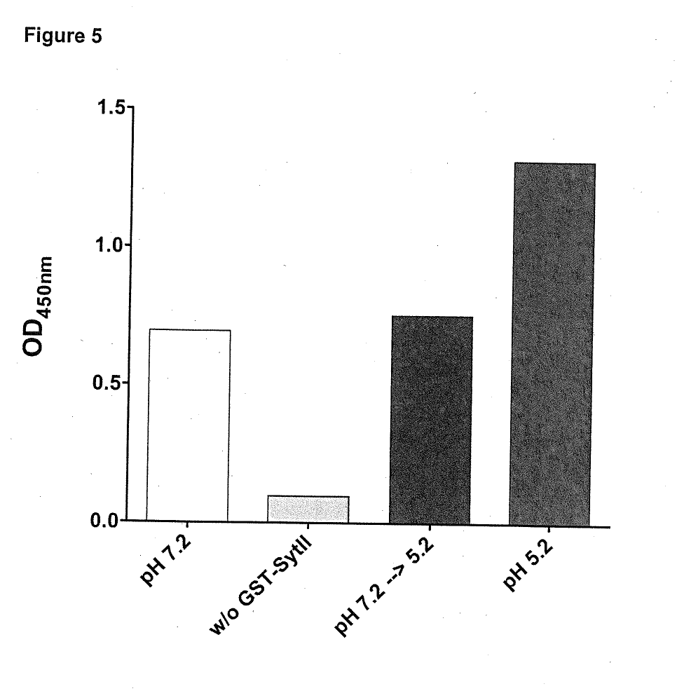

FIG. 5: Binding of BoNT/B to GST-SytII under different pH conditions. Binding of GST-SytII to BoNT/B coated on microtiter plates at physiological (pH 7.2) and at acidic pH (pH 5.2). In the positive control (2nd bar from the left) GST-SytII (100 μg/mL) was added to coated BoNT/B (500 ng/mL) and incubated at pH 7.2. In the negative control (1st bar from the left) no GST-SytII was added to coated BoNT/B. The 3rd bar from the left shows signal for GST-SytII, which was added to BoNT/B at pH 7.2, incubated for 60 minutes, the supernatant removed and the well incubated for 30 minutes at room temperature in buffer with pH 5.2. The 4th bar from the left shows signal for GST-SytII, which was incubated with BoNT/B at pH 5.2 for 90 minutes in total. Following incubation steps were again performed under standard conditions at pH 7.2.

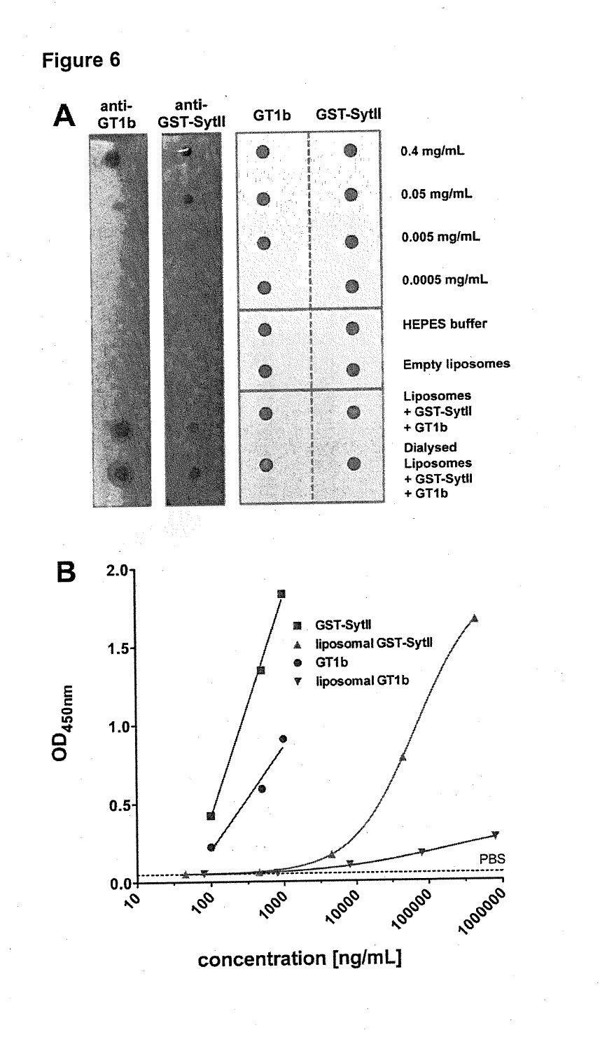

FIG. 6: Immunological detection of GT1b and GST-SytII integrated in liposomes. (A) Dot blot analysis of liposomes with and without GT1b and GST-SytII in comparison to diluted GT1b and GST-SytII standards as positive control and empty liposomes and buffer as negative control. (B) ELISA detection of GST-SytII and GT1b integrated into dialysed receptor liposomes (triangles and inverted triangles), in comparison to isolated GST-SytII (squares) and GT1b (circles). Results for PBS (negative control) are shown as dotted black line. Liposome were composed of 20 mM SPC/Cholesterol/D,L-alpha-tocopherol (77.1:19.5:0.63), 0.2 mM GT1b (1 mM GT1b corresponds to 2.129 mg/mL), and 0.45 mg/mL GST-SytII.

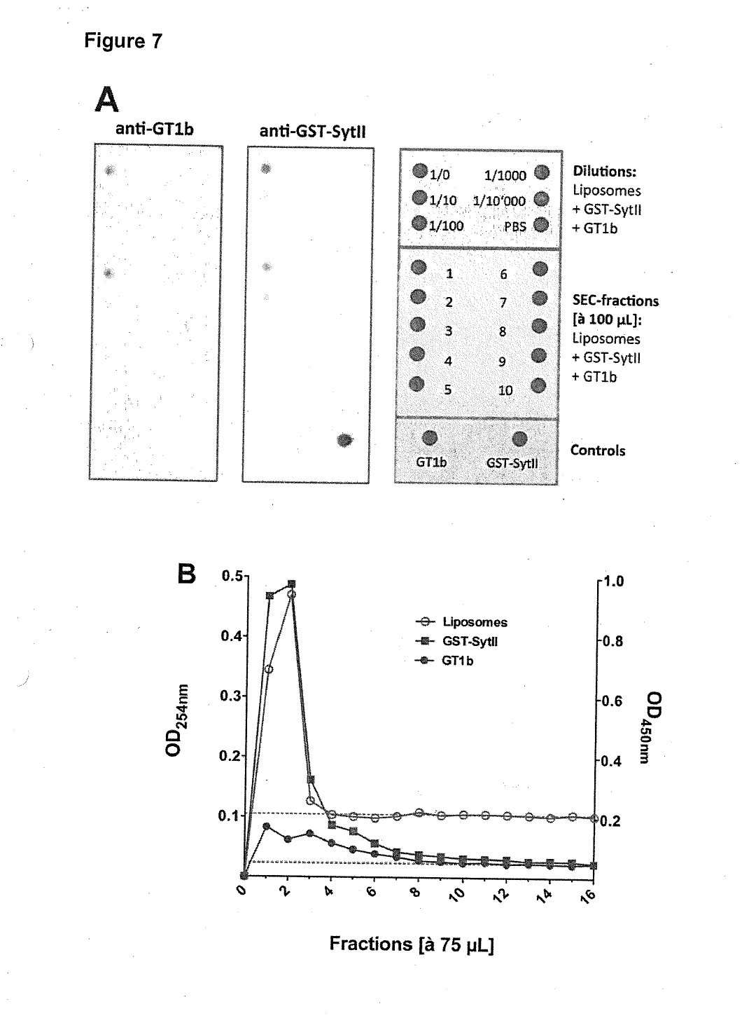

FIG. 7: Separation of receptor liposomes by SEC. (A) Dot blot of serially diluted non-separated receptor liposomes with GT1b and GST-SytII, and PBS as negative control (upper part), fractions of undiluted liposomes separated by SEC (middle), and positive controls, i.e. 0.02 mM GT1b and 7.2 mg/mL GST-SytII (lower part). (B) SEC Fractions of receptor liposomes analysed for presence of liposomes via their extinction at λ=254 nm (empty circles) and for presence of receptors, i.e. GST-SytII (squares) and GT1b (filled circles), respectively, via ELISA. The dotted upper line represents the negative control for OD254 (PBS), the dotted lower line (two lines coinciding) represent the negative controls for GST-SytII and Glib, respectively. Liposome were composed of (A) 20 mM SPC/Cholesterol (69.75:29.75), 0.2 mM GT1b, and 0.72 mg/mL GST-SytII and (B) 20 mM DOPC/Cholesterol (18:1), 0.2 mM GT1b, and 0.72 mg/mL GST-SytII.

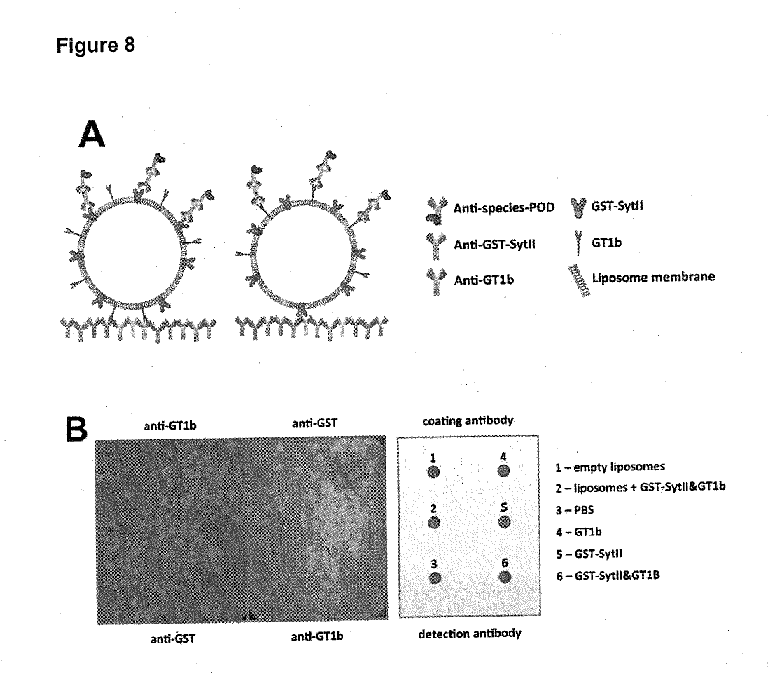

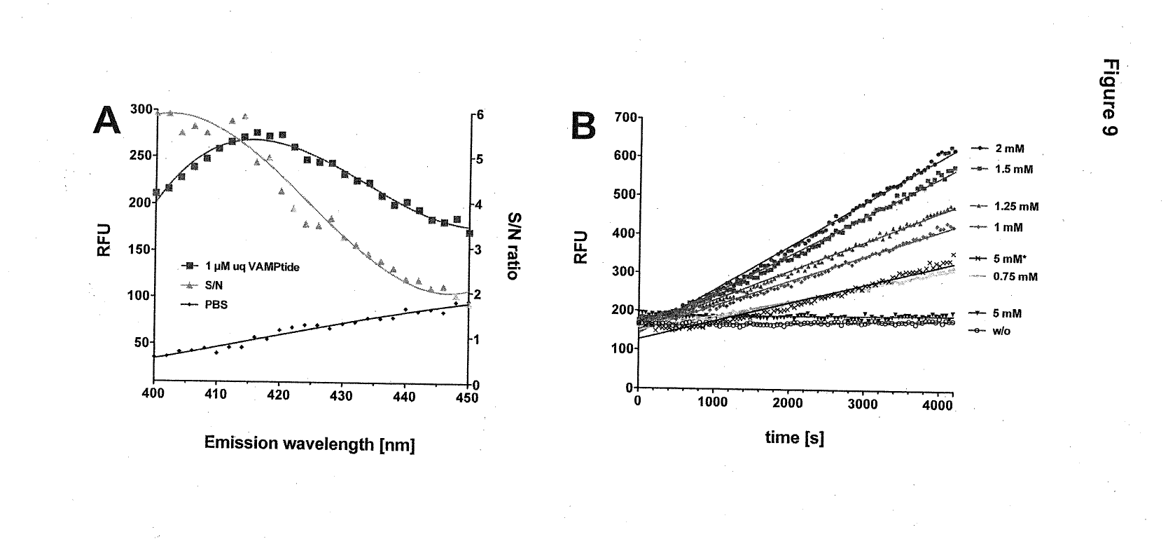

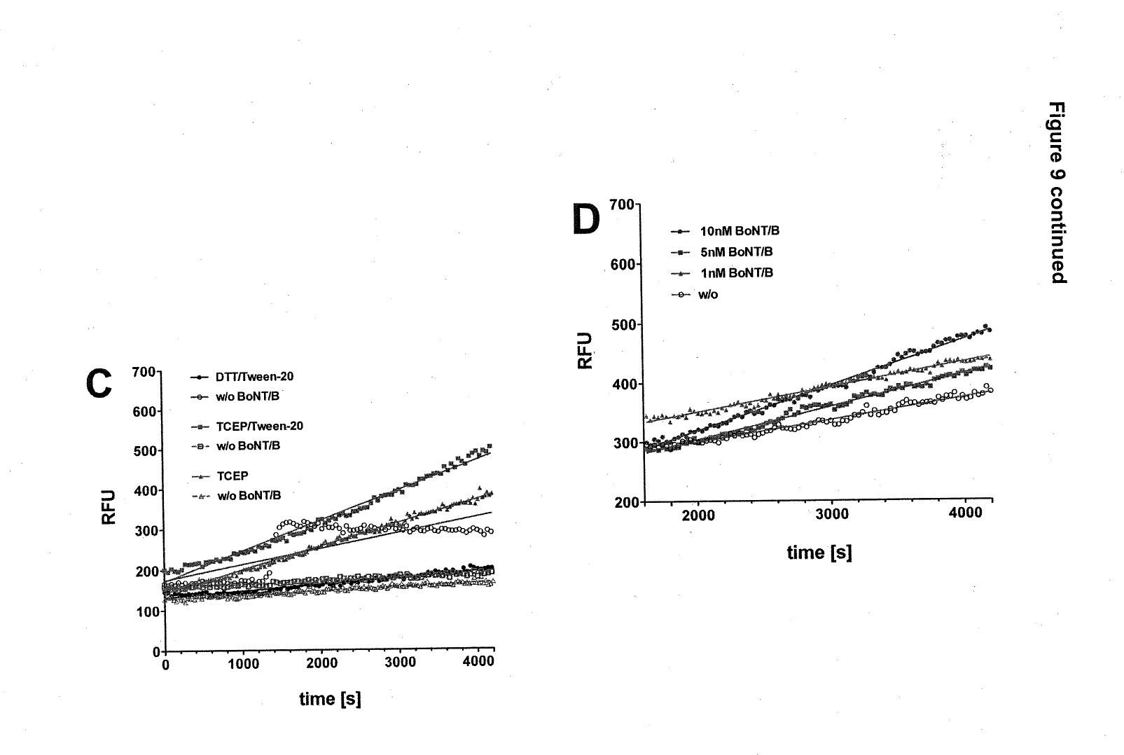

FIG. 8: Sandwich dot blot for detection of dual-receptor integration into liposomal membranes. (A) Illustrated model of sandwich immunoassay against receptors on the liposome membrane. (B) Sandwich dot blot with (1) empty liposomes, (2) receptor liposomes, (3) PBS as negative control, and (4) isolated GT1b and (5) isolated GST-SytII, and (6) mixture of isolated Glib and GST-SytII as positive controls. Sample composition was (1) 20 mM DOPC/Cholesterol (18:1), (2) DOPC/Cholesterol (18:1), 0.5 mM GT1b, and 0.72 mg/mL GST-SytII, (4&6) 0.1 mg/mL GT1b, and (5&6) 7.2 mg/mL GST-SytII, respectively.

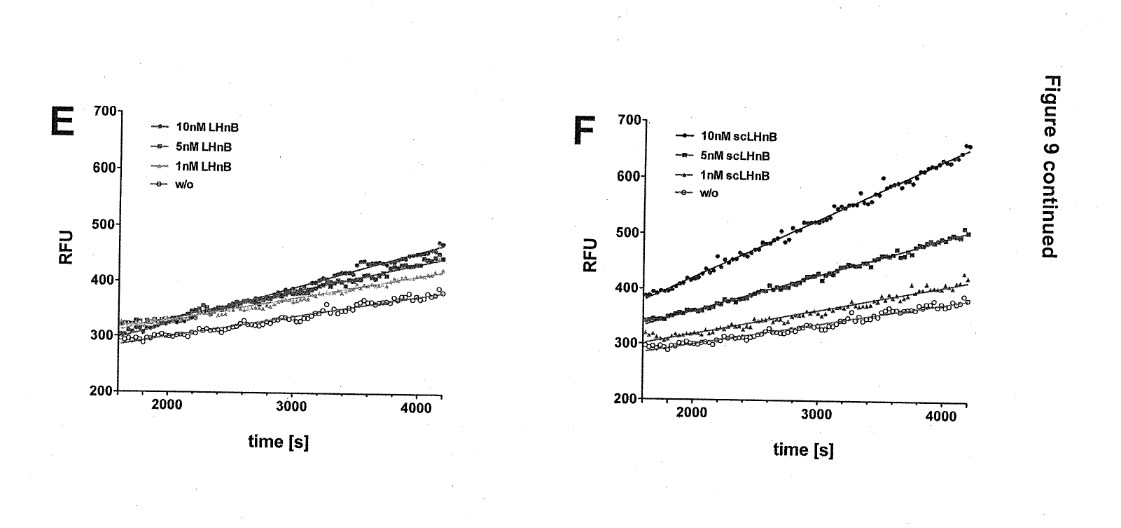

FIG. 9: VAMPtide fluorescence and cleavage. (A) Fluorescence spectrum (squares) of o-Abz in 1 μM uq VAMPtide (VAMPtide unquenched calibration peptide) between 400-450 nm (at an excitation wavelength of λ=321 nm) was recorded against a negative control with respective buffer (diamonds) and the corresponding S/N-ratio (triangles) calculated. (B & C) Cleavage of 10 μM VAMPtide with standard cleavage buffer (20 mM HEPES pH 7.4, 0.05 mM ZnSO₄) with TCEP or DTT as reducing agent, and with or w/o Tween-20, respectively. In (B) different concentrations of TCEP (0.75 to 5 mM) or 5 mM DTT (asterisk) were added to the standard cleavage buffer and cleavage of VAMPtide, tested with 20 nM BoNT/B. Cleavage buffer without toxin and without TCEP was used as negative control (open circles). (C) Cleavage activity of 10 μM VAMPtide by 10 nM BoNT/B was tested in standard buffer supplemented with 5 mM DTT & 0.2% Tween-20 (filled circles), with 2 mM TCEP & 0.2% Tween-20 (filled squares), and with 2 mM TCEP without Tween-20 (filled triangles), respectively. The respective cleavage buffers without BoNT/B (corresponding open symbols) were used as negative controls. (D-F) Modified cleavage buffer (20 mM HEPES pH 7.4, 0.05 mM ZnSO₄, 2 mM TCEP) was used to detect cleavage of 10 μM VAMPtide by different concentrations of (D) BoNT/B (filled symbols), (E) LH_{N}B (filled symbols), and (F) scLH_{N}B (filled symbols). Modified cleavage buffer without toxin (open circles) was used as negative control.

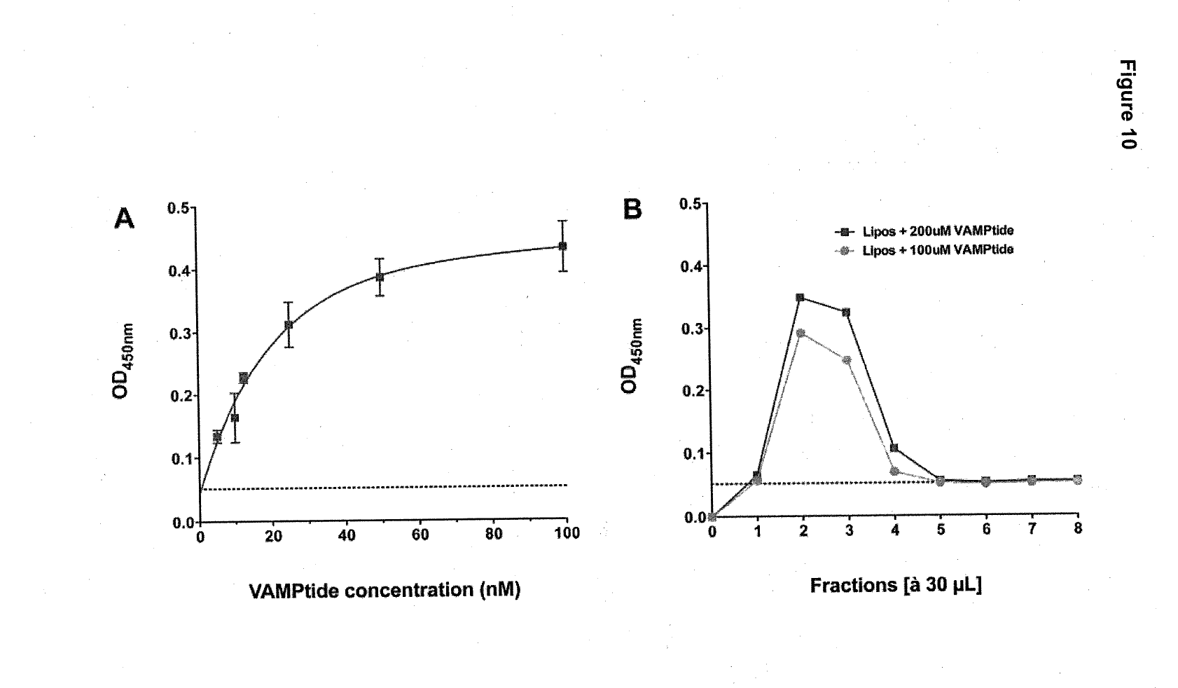

FIG. 10: Sensitivity of VAMPtide immunoassay and encapsulation efficiency of VAMPtide into liposomes. (A) Sensitivity of ELISA immunoassay with VAMP antibody and appropriate POD-coupled anti-species antibody. (B) Liposomes loaded with 200 μM (squares) and 100 μM VAMPtide (circles), respectively, were separated by SEC. (C) Comparison of encapsulation in fractions 1-5 with dilutions of unseparated VAMPtide loaded liposomes. (D) Standard curve of VAMPtide measured in different dilutions of unseparated VAMPtide liposomes. Liposomes were composed of (B & C) 20 mM DOPC/Cholesterol (18:1). For detection in (B & C), samples were diluted 1/2000 prior to coating on the ELISA-plate. Results for PBS (negative control) are shown as dotted line.

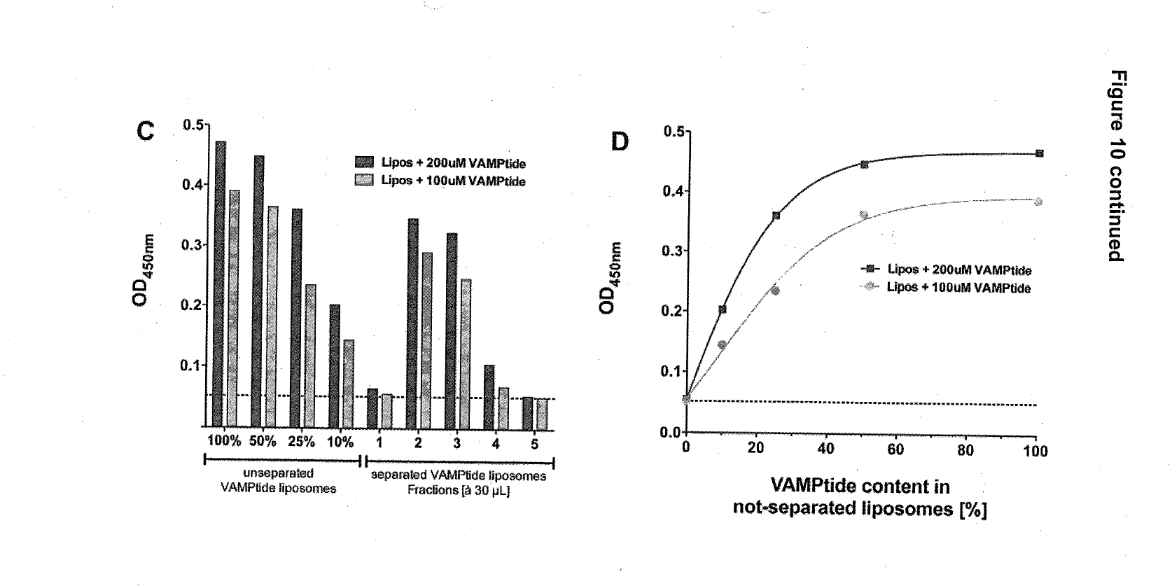

FIG. 11: Test for non-specific membrane binding of VAMPtide with protease protection assay. (A) Cleavage of VAMPtide (200 μM) by Proteinase K (100 μg/mL) detected by ELISA (OD450) with VAMP antibody (grey bar), and by fluorescent measurement (RFU) with Excitation/Emission at 321/418 nm (black bar). Samples were not diluted prior to coating on the ELISA-plate. Results for PBS as negative control are shown as dotted grey and dotted black line for ELISA and RFU measurement, respectively. (B) VAMPtide liposomes, which were digested without (squares), with 50 μg/mL (circles), and with 100 μg/mL (triangles) Proteinase K, respectively, were separated by Micro-SEC and uncleaved VAMPtide detected by ELISA with VAMP antibody. Samples were diluted 1/800 in PBS prior to coating on the ELISA-plate. Liposome composition for was 20 mM DOPC/Cholesterol (18:1), 200 μM VAMPtide. Results for PBS (negative control) are shown as dotted line.

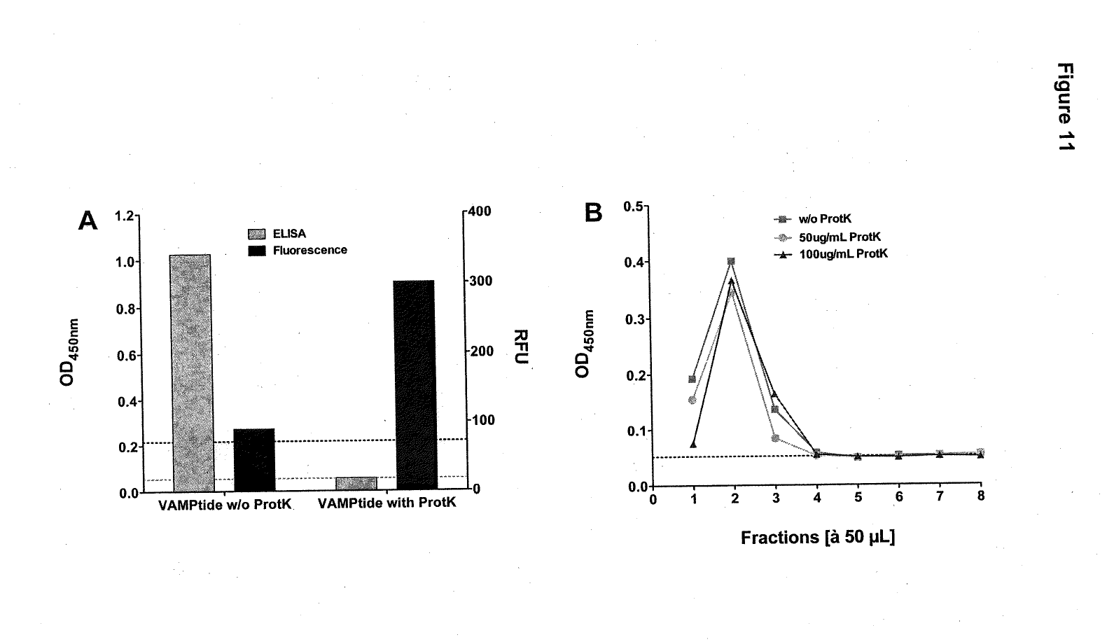

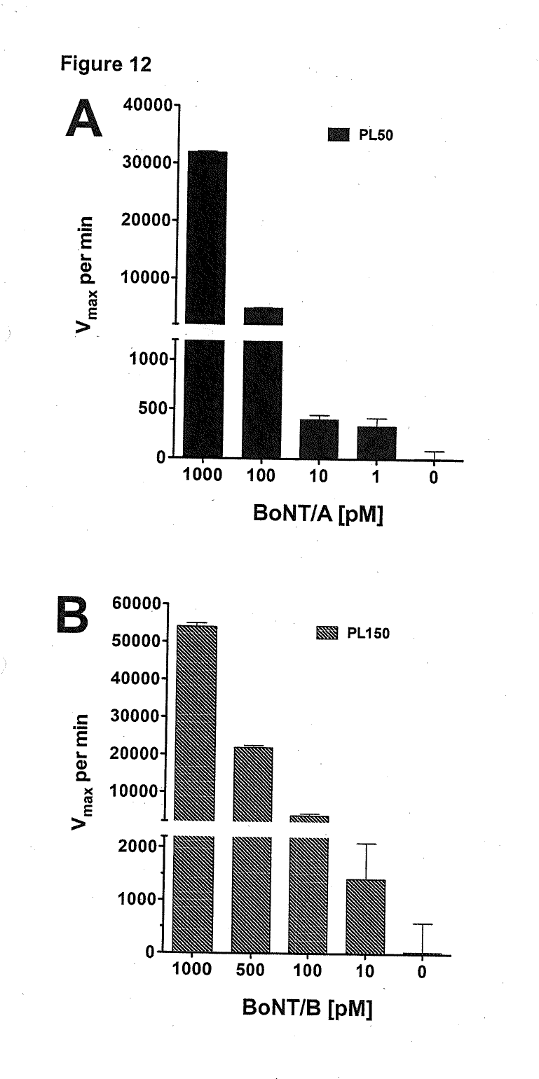

FIG. 12: Sensitivity of Pharmaleads cleavage substrates for BoNT/A and BoNT/B. (A) Signals of PL50 in RB_{PL50} cleaved by 1000, 100, 10 and 1 μM BoNT/A and (B) PL150 in RB_{PL150} cleaved by 1000, 500, 100 and 10 μM BoNT/B were recorded over 2 h at 37° C. against a negative control without any toxin (0 pM). Sensitivity is shown as V_{max per min}. PL50 and PL150 were used at an assay concentration of 10 μM each.

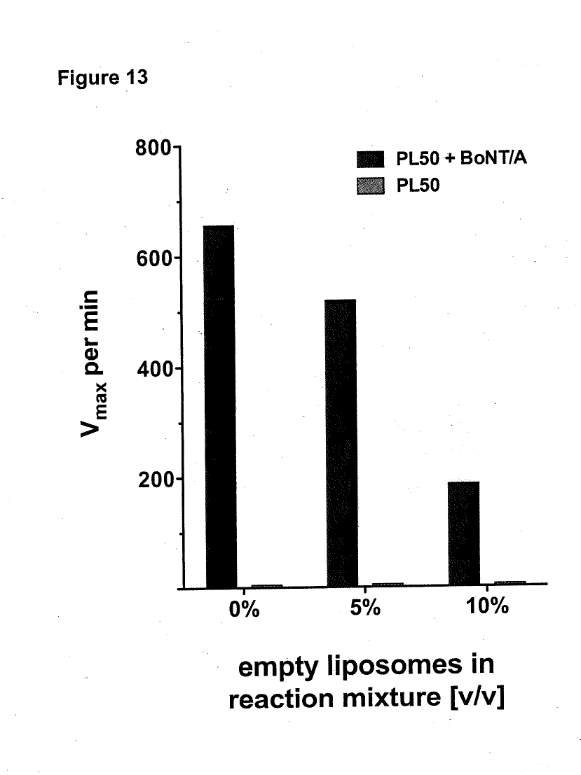

FIG. 13: Cleavage of PL150 substrate by BoNT/A in the presence of liposomes. (A) Signals of PL50 in RB_{PL50} and 0, 5, or 10% (v/v) liposomes (40 mM DOPC/Cholesterol) cleaved by 10 pM BoNT/A were recorded over 2 h at 37° C. against a negative control without any toxin. Sensitivity is shown as Vmax per min. The liposomes were diluted in RB_{PL50}. Negative controls contained no toxin.

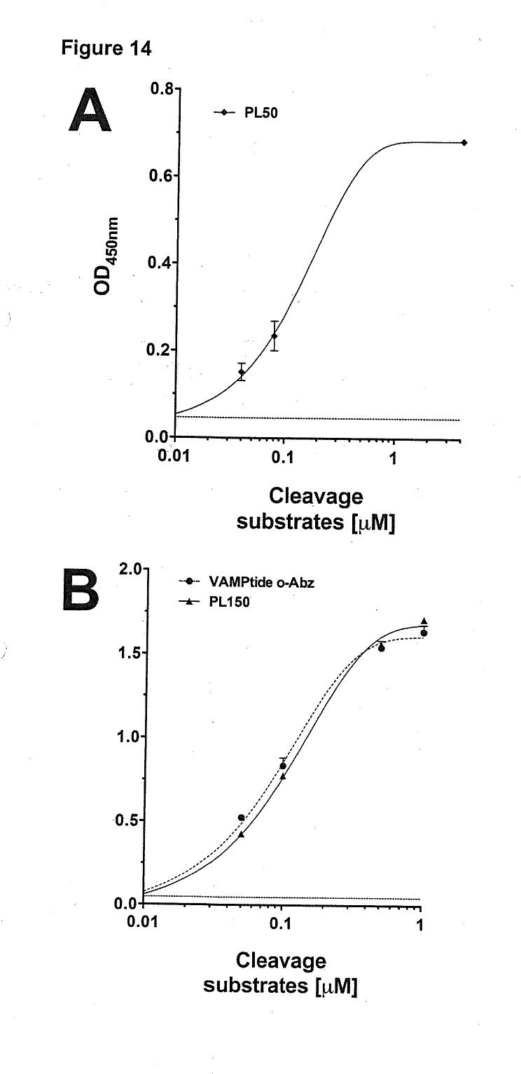

FIG. 14: Immunological detection of BoNT/A and BoNT/B cleavage substrates. (A) PL50 as cleavage substrate for BoNT/A was detected with rabbit@SNAP-25 antibody and (B) PL150 and VAMPtide, respectively, as cleavage substrate for BoNT/B were detected with rabbit@VAMP1,2,3 antibody. The negative control (no cleavage substrate coated) is depicted as dotted line.

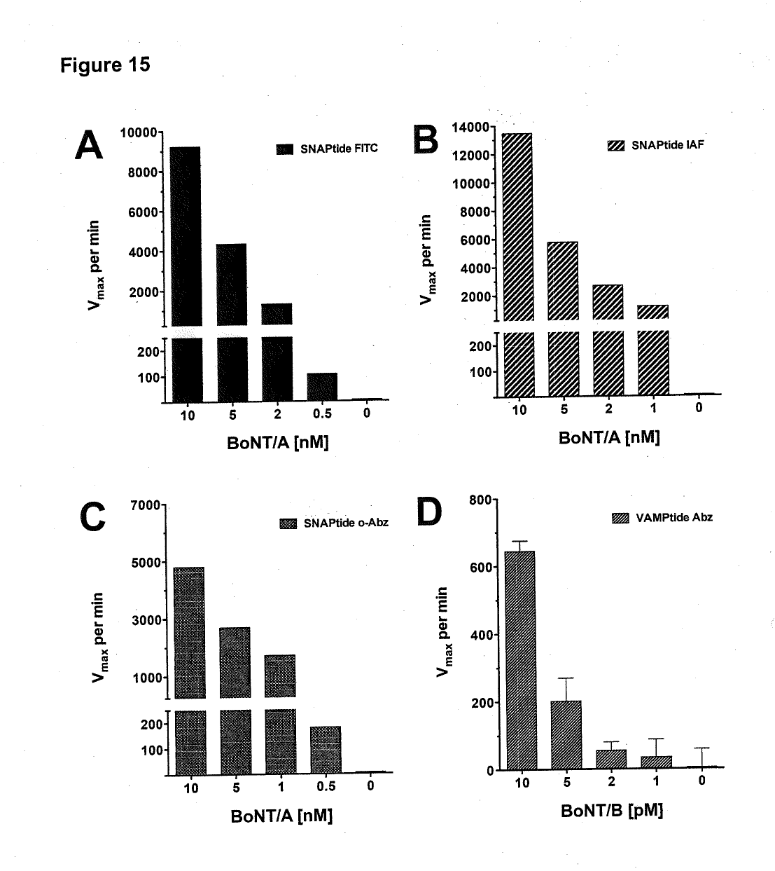

FIG. 15: Sensitivity of List Biological Laboratories cleavage substrates for BoNT/A and BoNT/B. Signals of substrates cleaved by different concentrations of BoNT/A in the respective reaction buffer are depicted in (A-C). (D) Signal for VAMPtide_{o-Abz}, cleaved by different concentrations of BoNT/B. Signals were recorded over 2 h at 37° C. against a negative control without any toxin (0 nM). Sensitivity is shown as V_{max per min}.

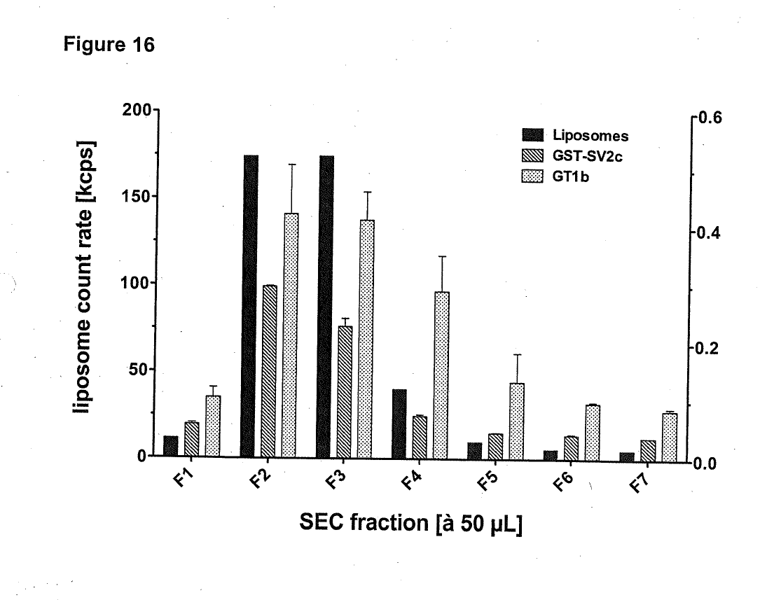

FIG. 16: Separation of A-liposomes by SEC. SEC fractions of A-liposomes analysed for liposomes via the count rate determined by dynamic light scattering (black bars) and for receptors, i.e. GST-SV2c (shaded bars) and GT1b (dotted bars), respectively, via ELISA. A-liposomes were composed of 40 mM DOPC/Cholesterol, 0.2 mM GT1b, and 0.0125 mg/mL GST-SV2c.

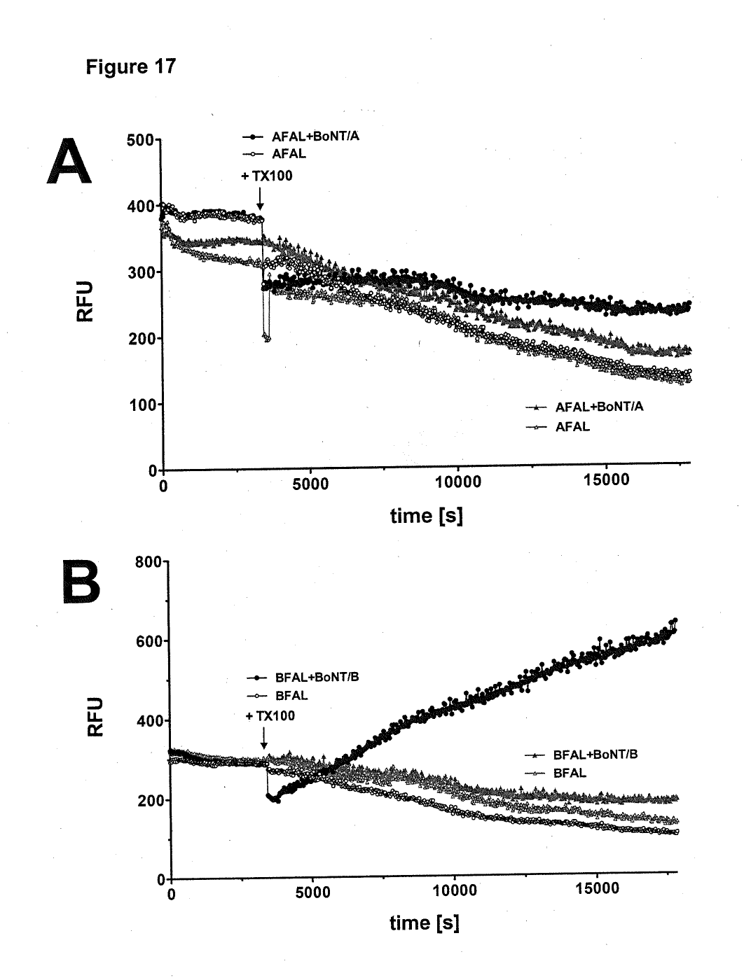

FIG. 17: Release of cleavage substrate upon lysis of A-FAL and B-FAL. Effect of lysis of (A) A-FAL or (B) B-FAL by addition of 0.1% Triton X-100 in the presence (filled black circles) or absence (white circles) of 100 pM BoNT/A or BoNT/B, respectively. No Triton X-100 was neither added to the controls, containing A-FAL or B-FAL and 100 pM BoNT/A or BoNT/B, respectively (filled grey triangles), nor to the negative controls, only containing A-FAL or B-FAL (white triangles). FAL were composed of 40 mM DOPC/Cholesterol, 0.2 mM GT1b and either 0.008 mg/mL GST-SV2c and 200 μM PL50 (for A-FAL) or 0.72 mg/mL GST-SytII and 200 μM PL150 (for B-FAL) before treatment with BioBeads SM-2 and dialysis. The reactions was performed in 10 mM HEPES, supplemented with 75 μM ZnSO₄.

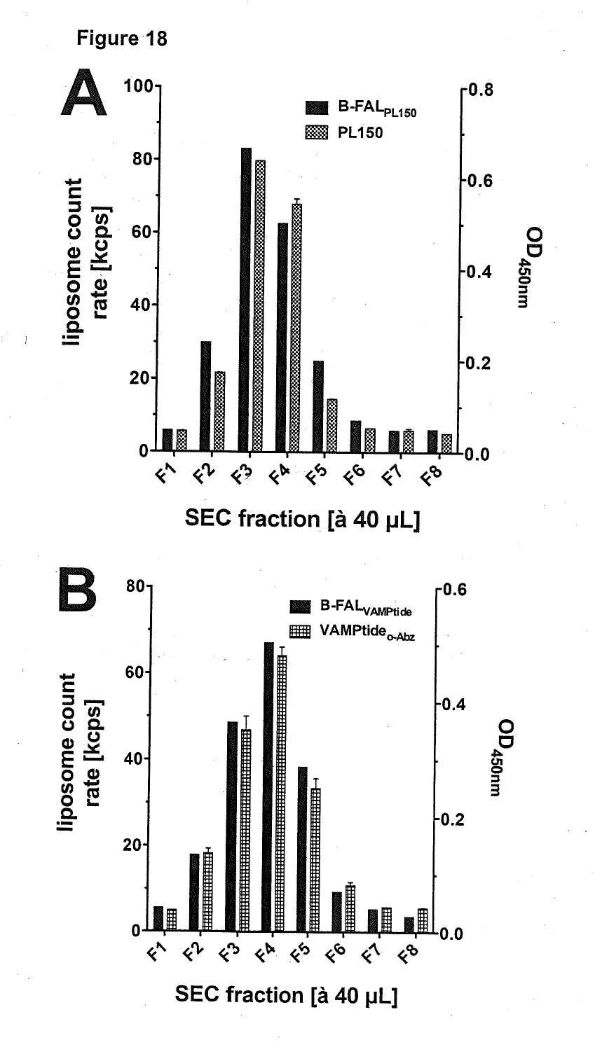

FIG. 18: Cleavage substrate in B-FAL SEC-fractions. SEC fractions of B-FAL, analysed for liposomes via the count rate determined by dynamic light scattering (black bars) and for (A) PL150 and (B) VAMPtide_{o-Abz} cleavage substrate for BoNT/B (chequered bars), respectively, via ELISA. B-FAL were composed of 40 mM DOPC/Cholesterol, 0.2 mM GT1b, 0.72 mg/mL GST-SytII and 200 μM BoNT/B cleavage substrate before treatment with BioBeads SM-2 and dialysis. For detection of PL150 and VAMPtide_{o-Abz}, SEC fraction were diluted 1/2000 in 10 mM HEPES buffer containing 0.1% Triton X-100 prior to coating on ELISA microplates at 4° C. over night.

The examples illustrate the invention but are not to be construed as being limiting.

### EXAMPLE 1

### Material and Methods

### Botulinum Neurotoxin Preparations

The study was performed using purified 150 kDa BoNT/B and BoNT/B complex from C. botulinum strain Okra B (Metabiologics Inc., Madison, Wis., USA), as well as isolated BoNT/B (LC=residues 1-441), BoNT/B LH_{N}B and BoNT/B scLH_{N}B (BoNT/B truncation mutants consisting of residues 1 to 863 and lacking the H_{C}-fragment; either as single polypeptide chain (sc) or dichain hydrolysed into LC and HC after R441; both available from A. Rummel, Hannover Medical School (MHH), Institute for Toxicology). The latter two represent toxin derivatives derived from directed mutagenesis devoid of the receptor-binding domain of BoNT/B. Also, scLHnB is still connected to the BoNT/B LC not only via a disulphide, but also via a peptide bond.

### Nerve Cell Receptors

Lyophilised Trisialoganglioside GT1b from bovine brain with a total mass of 2180 Da was purchased from Merck KGaA (Darmstadt, Germany). The ganglioside was dissolved in a 2:1 mixture of Chloroform (CHCl₃) and methanol (CH₃OH) and stored at −20° C.

Instead of 422 amino acids found in the native SytII protein, the modified version used here presents is truncated, containing 61 amino acids of the N-terminus, with 30 amino acids representing the transmembrane domain. For purification, after production in E. coli BL21, the truncated SytII also contains GST (Glutathione S-transferase) coupled to the N-terminus, without hindering binding of BoNT/B H_{C}. GST-SytII is bound in Triton X-100 micelles due to the purification protocol used by Andreas Rummel's group (Rummel et al. [80]).

For immunoassay experiments, mouse anti-GT1b antibodies (Millipore, Billerica, Mass., USA) or rabbit polyclonal anti-GST antibody (Bethyl Laboratories Inc., Montgomery, Tex., USA) at a concentration of 0.1 μg/mL, and peroxidase coupled goat anti-mouse, or goat anti-rabbit antibodies (Sigma-Aldrich, Buchs, Switzerland) at 1/1000 dilution were used. TMB (3, 3′,5, 5′-tetramethylbenzidine; Sigma-Aldrich, Buchs, Switzerland) or 4CN (4-chloro-1-naphthol; Bio-Rad, Laboratories Inc., Hercules, Calif., USA) were used as substrates for ELISA or dot blot experiments, respectively.

### VAMPtide

VAMPtide (List Biological Laboratories, Inc., Campbell, Calif., USA) was used as a peptide substrate for measuring proteolytic activity of BoNT/B LC. VAMPtide is an oligopeptide with o-Abz (o-Aminobenzoic acid) as donor fluorophor coupled to the N-terminus, and DNP (2,4-Dinitrophenyl) as acceptor/quencher with an absorbance spectrum overlapping the emission spectrum of the donor fluorophor coupled to the C-terminus. In between, the peptide contains a recognition sequence specific for the cleavage by BoNT/B LC. In the intact, uncleaved state, Forster resonance energy transfer (FRET) between the donor fluorophor and the quencher impedes emission of fluorescence. If VAMPtide is cleaved by BoNT/B LC, then donor fluorophor and quencher are spatially separated and, if excited, the donor fluorophor emits quantifiable fluorescence.

For immunological detection of uncleaved VAMPtide molecules, a 1/1000 dilution of polyclonal rabbit antibody generated against aa 1-81 of the cytoplasmic part of rat cellubrevin (anti-VAMP 1/2/3; Synaptic Systems GmbH, Göttingen, Germany) was used. Binding of the anti-VAMP antibody was detected via peroxidase coupled anti-rabbit antibody (Sigma-Aldrich, Buchs, Switzerland).

VAMPtide assay

Unless otherwise stated VAMPtide assay for the detection of BoNT cleavage activity was performed according to the manufacturer's instructions. In short, lyophilised VAMPtide was resuspended in DMSO resulting in a 5 mM stock solution and stored at −20° C. Prior to the experiments, stock solution was diluted in 20 mM HEPES to 250 μM. The reaction buffer for hydrolysis of VAMPtide by BoNT/B was 20 mM HEPES (pH 7.4), 0.05 mM ZnSO4, 5 mM DTT and 0.2% Tween-20. For reactions with BoNT/B LC the hydrolysis buffer contained 50 mM HEPES (pH 6.3) and 0.05% Tween-20. For the assays a final concentration of 10 μM VAMPtide was used. Assays were performed in black FluoroNunc F96 Micro-Well plates (Nunc, Langenselbold, Germany). The assay was run at 37° C. in a Spectramax Gemini XS fluorescence microplate reader (Molecular devices, Sunnyvale, Calif., USA) with an excitation wavelength of 321 nm and emission wavelength at 418 nm.

### Lipids and Liposomes

### Lipids Used for Liposome Production

For production of receptor liposomes either a mixture of SPC (soy Phosphatidylcholine; Phospholipon 85G; Lipoid A G, Cham Switzerland), cholesterol (Sigma-Aldrich, Buchs, Switzerland), and DL-alpha-tocopherol (VW R, Dietikon, Switzerland) or a predefined mixture of DOPC (1,2-Dioleoyl-sn-glycero-3-phosphocholine) or POPC (1-Palmitoyl-2-oleoyl-sn-glycero-3-phophocholine) and cholesterol (Avanti Polar Lipids Inc., Alabaster, USA) was used. DL-alpha-Tocopherol was supplemented for its antioxidant and membrane stabilising effect [36].

An alternative lipid mixture may be prepared as follows. Soybean asolectin, dioleoyl L-K-phoshatidylcholine, bovine brain phospholipids or alternatively a phosphatidic acid (PA) lipid mixture (consisting of dioleoyl L-alpha-phosphatidylcholine 70%, phosphatidic acid 20% and cholesterol 10%) are dissolved in chloroform, dried to a thin film under a gentle N₂ flow and vacuum pumped for at least 2 h to remove residual traces of organic solvent.

### Production of LUVs (Large Unilamellar Vesicles)

For LUV production the required volume of the respective lipid mixture was dissolved in a 1:1 mixture of methanol (CH₃OH) and dichloromethane (CH₂Cl₂), and dried at room temperature under vacuum at 1400 rpm in a Eppendorf concentrator 5301 with a F 45-48-11 Rotor (Eppendorf A G, Hamburg, Germany) until complete evaporation of the organic solvents (1-2 h). Upon complete evaporation of solvent, the lipids formed a thin yellow film on the walls of the vials. For longer storage, vials were sealed under N₂-atmosphere and stored at −20° C. For liposome production, the lipid film was resuspended in appropriate volumes of 20 mM HEPES buffer (pH 7.4) as aqueous medium. The vial was shaken until complete solution of the lipids, giving a turbid white emulsion of multilamellar vesicles (MLV). Examination under Zeiss Dialux 20 EB light microscope using 100-fold object lens (100/1.25 oil immersion) plus 10 fold ocular (Periplan GF 10×/18) magnification allowed for visual control that the MLV had been formed. The vial with the emulsion was shaken with 1400 rpm in an Eppendorf Thermomixer comfort (Eppendorf A G, Hamburg, Germany) at 30° C. for 30 minutes until the lipid film was completely dissolved in the aqueous medium. LUVs were obtained by extrusion through polycarbonate (PC) membranes (Nucleopore track-etched polycarbonate membrane; Whatman plc, Maidstone, UK) with defined pore sizes (400, 200, and 100 nm) in a MiniExtruder (Avanti Polar Lipids Inc., Alabaster, Ala., USA). The equipment was assembled according to the manufacturer's instructions and, for production of LUVs, the MLV emulsion was passed through a 400 nm, and subsequently through 200 and 100 nm pore size PC-membrane. Homogeneity and size were controlled via DLS (Dynamic Light Scattering) with a Brookhaven Instruments BI-200SM research goniometer and particle sizer (Brookhaven Instruments, Holtsville, N.Y., USA) at an angle of 90° at 25° C. sample temperature. Data was processed with Brookhaven Instruments Particle Sizing Software (Brookhaven Instruments, Holtsville, N.Y., USA).

While LUVs are preferred, liposomes may also be prepared as follows.

### Production of DRVs (Dehydration-Rehydration Vesicles)

VAMPtide or other any other substrate according to the invention is diluted in the reaction buffer (preferably 20 mM HEPES with ZnSO₄ and TCEP at pH 7.2-7.4 as defined herein above; see “aqueous medium”) at concentrations of 100-200 μM and added to a lipid film giving an emulsion of 40 mM DOPC/Cholesterol (13/1), 100-200 μM substrate in the reaction buffer. The emulsion is extruded eleven times each through polycarbonate membranes with pore diameters of 200 nm and 100 nm (and, if preferred, 50 nm) pore diameter. Then, lactose or trehalose is added to give a final concentration of 1 to 5%, preferably 3.5%. In the following, the (clear) liposome solution is cooled to 4° C., to −20° C., and subsequently to −80° C. The frozen emulsion is then subjected to freeze-drying over night until complete evaporation of the contained liquid. Reconstitution is carried out first by adding 1/10 of the original liquid volume as 10 times concentrated buffer (preferably 10×HEPES). For resuspension, the lyophilizate plus added buffer is left for 30 minutes at room temperature, and then vigorously shaken, centrifuged, and again vigorously shaken. Subsequently, liquid (i.e. distilled H₂O) is added till the original level of liquid volume is reached.

### Production of Sonicated Vesicles

VAMPtide or other any other substrate is diluted in the reaction buffer (preferably 20 mM HEPES with ZnSO₄ and TCEP at pH 7.2-7.4 as defined herein above; see “aqueous medium”) at concentrations of 100-200 μM and added to a lipid film giving an emulsion of 40 mM DOPC/Cholesterol (13/1), 100-200 μM substrate in the reaction buffer. The vessel containing the emulsion is placed into a 0° C. waterbath. The transducer tip of the sonicator (such as Branson sonifier) is immersed into the sample. Different durations and sonication times can be applied until the emulsion turns from milky to opalescent. Metallic particles from the transducer tip can be removed by centrifugation.

### Liposome Separation Via Size Exclusion Chromatography

Unless otherwise stated, liposome emulsions were subjected to SEC (size exclusion chromatography) columns with a gel bed volume of approximately 600 μL for separation from not-integrated and/or not-encapsulated compounds. SEC columns allowed for easy and quick separation of 10-100 μl of sample via centrifugation in a tabletop centrifuge. Due to the small gel-volume the dilution effect was relatively low (1-2 times dilution). The technique works as common gravitational SEC-column, just that the gravitational force is replaced by centrifugal force. The gel, in this case a 50%-slurry of Sepharose 4B (Sigma-Aldrich, Buchs, Switzerland) in 20 mM HEPES (pH 7.4), is filled into the column, i.e. a 0.5 mL Eppendorf cup pierced on the bottom and stuffed with 1 mm glass fibres. For separation, 10-100 μL of the sample are loaded slowly onto the gel bed, assuring that none of the sample passes along the sides of the SEC column. Centrifugation of the tube at 1200 RCF for 45 s elutes the first fraction of the sample, including the components with the biggest hydrodynamic volume. Following, further application of elution buffer with the same volumes as the sample applied and further centrifugation steps elute further fractions with decreasingly smaller hydrodynamic volume. The arrangement of the system is shown in FIG. 2. For quantification of liposomes separated by SEC, optical density (light scattering effect) of the solution is determined at λ=254 nm.

### Production of Receptor and VAMPtide-Liposomes

For production of receptor liposomes, GT1b was added to the lipid mixture dissolved in organic solvent and either dried under vacuum or under constant laminar flow of gaseous N₂. Due to the high transition phase of GT1b gangliosides, the lipid solution cannot be dried out completely. Accordingly, instead of a lipid film the lipid mixture is present in a highly viscous gel-like state. For complete solvation of the gel, the whole suspension is pipetted up and down in aqueous medium and subsequently shaken as described above. GST-SytII is added with the aqueous medium in the respective concentration. Likewise, for encapsulation experiments, VAMPtide is added with the aqueous medium in the respective concentration to dried lipid film. To remove Triton X-100 from liposomes with GST-SytII, the emulsion is incubated with pre-hydrated Bio-Beads SM-2 Adsorbents (Bio-Rad, Laboratories Inc., Hercules, Calif., USA). Bio-Beads plus GST-SytII liposomes are incubated under rotation at 4° C. for 2.5 h. Thereafter, another portion of SM2 beads is added and incubated with the emulsion for another 2 h. To separate Bio-Beads with bound Triton X-100 from the liposome, the mixture is kept still for 5 min to allow settling of the beads. After transferring the liposomes in the supernatant to a clean vial, the remainder is centrifuged at 5.000×g RCF for 30 seconds and the resulting supernatant pooled with the first one.

### Immunoassays

For ELISA experiments, receptors, VAMPtide, receptor liposomes, or VAMPtide liposomes are coated on MaxiSorp microtiter plates (Nunc, Langenselbold, Germany) at 4° C. overnight. After 60 min of blocking, sample cavities are washed and incubated for 60 min with the respective antibodies, followed by incubation with species-specific peroxidase coupled antibodies for 30 min.

In direct dot blot experiments, two times 1 μL of the antigen is pipetted onto nitrocellulose membranes (Bio-Rad, Laboratories Inc., Hercules, Calif., USA) and dried at ambient temperature. After 60 min of blocking, membranes are washed and incubated for 45 min with the respective antibodies, followed by incubation with species-specific peroxidase coupled antibodies for 30 min. In sandwich dot blot experiments, entire membranes are coated with the respective capture antibody for 60 min. After washing, membranes are blocked overnight at 4° C. Then, membranes are washed, dried slightly, subsequently 1 μL of the respective antigens pipetted onto the membrane and incubated in a humid chamber for 60 min. Afterwards, membranes are incubated with detection antibody complementary to the coating antibody for 45 min, followed by 30 min incubation with peroxidase coupled antibody, binding the respective detection antibody.

### EXAMPLE 2

### Characterisation of Liposomes

The aqueous liposomes lumen provides a cell-like reaction compartment. Due to the multiple and complex reactions (binding of BoNT/B, translocation of BoNT/B LC and VAMPtide cleavage) that are supposed to take place in the assay, preferably LUV with a size of approximately 100-200 nm are used to provide for a sufficiently large reaction volume. For the reproducibility and functionality of the assay it is preferable that the liposome emulsion were consistent concerning their lamellarities and diameters. Accordingly, emulsions with mainly MLVs obtained by reconstitution of lipid films were subjected to an extrusion process. We used an Avanti MiniExtruder combined with PC membranes with pore sizes between 400 and 100 nm to obtain homogenous emulsions with liposome diameters between 100 and 200 nm. Success of the extrusion process was monitored by visible control with (light microscopy; not shown) or without magnification, as shown in FIG. 3B. While liposome emulsions extruded through 400 nm pore diameter PC membranes did not change notably, continuous extrusion through 100 nm PC membranes increased the translucence of the emulsion. DLS was used to receive exact information on size distribution and homogeneity of the extruded sample. FIG. 3C exemplarily shows estimates of the distribution of liposome diameters in a liposome emulsion from 20 mM lipids (SPC/Cholesterol/Tocopherol). After eleven times extrusion through 400 nm and subsequently eleven times extrusion through 100 nm pore diameter PC membranes, the ratio of liposome diameters measured in the emulsion (intensity) resembled a Gaussian distribution.

Careful review of current literature suggested that artificial lipids might present an alternative to natural phosphatidylcholine [37-39,35]. Especially for reliable imitation of the characteristics of eukaryotic cell membrane it is preferred to use lipids such as DOPC (1,2-Dioleoyl-sn-glycero-3-phosphocholine) or POPC (1-Palmitoyl-2-oleoyl-sn-glycero-3-phosphocholine), due to their low transition phase temperature (approximately 0° C.) and their structural properties (Steven Burgess, director R&D, Avanti Polar Lipids, personal communication). Accordingly, not only SPC, but also DOPC, both in combination with Cholesterol, were tested. DLS measurements were performed for the liposomes derived from different lipid formulations (Table 2).

TABLE 2
Characteristics of liposome emulsions from different lipid formulations. Mean diameter
and calculated lipid molecules per liposome and liposomes per mL, respectively.

| Lipid formulation | Lipid | | Mean | | |
|---|---|---|---|---|---|
| molar ratio | concentration | Mean a* | diameter** | N_{lipid} | Nₗᵢₚₒ |
| SPC | 20 mM | 0.700 nm² | 148.8 nm | 1.86 × 10⁵ | 64.79 × 10⁹ |
| SPC/Cholesterol/Tocopherol | 20 mM | 0.574 nm² | 157.2 nm | 2.54 × 10⁵ | 47.44 × 10⁹ |
| 69.9:29.9:0.2 | | | | | |
| DOPC/Cholesterol | 20 mM | 0.697 nm² | 170.4 nm | 2.47 × 10⁵ | 97.56 × 10⁹ |
| 18:1 | | | | | |

*a = head group area, calculated on the basis of the molar ratio of the lipid formulation and on 0.70 nm², 0.28 nm², and 0.72 nm² as the average lipid head group areas for SPC, Cholesterol and DOPC at 30° C., respectively.
**mean diameter calculated out of multiple experiments.

The quantity of lipid molecules per liposome (N_{lipid}) was calculated with formula (1), using the average diameter of the lipid head groups (a), the outer liposome diameter (d), and the inner liposome diameter, which is the outer diameter reduced by 5 nm (the average thickness of a lipid double membrane).

N_{lipid}=4π×1/a×[(d/2)²+(d/2−5 nm)²] (1)

The total number of liposomes (N_{lipid}) in one millilitre of the respective emulsion was calculated using formula (2), with the concentration of the lipid mixture in Mol (c_{lipid}), the Avogadro constant of 6.022×10²³ mol⁻¹ (N_{A}) and division by 1000 for conversion from L to mL [40].

N_{lipid}=(c_{lipid}×N_{A})/(Nₜₒₜ×1000) (2)

Interestingly, average diameters were higher in DOPC liposomes in comparison to SPC liposomes, although the head groups of both lipids occupy almost the same average area. Lower cholesterol content in DOPC could not have been responsible, as SPC liposomes without any cholesterol were even smaller. An explanation for differing liposome sizes could be, that DOPC is known to have a higher bilayer bending rigidity when compared with similar lipids [41-43]. Hence, extrusion with the MiniExtruder was not able to reduce average diameters of DOPC liposomes below 170 nm. The extruded liposomes from the three formulations shown in Table 2 showed low polydispersity, i.e. high homogeneity, were easily extruded and matched the required size of 100-200 nm. Hence, both liposome types were used in the following experiments.

### Detection and Liposome Integration of BoNT/B Nerve Cell Receptors

### Immunological Detection of GT1b and GST-SytII

In order to detect the nerve cell receptors GST-SytII and GT1b, either in solution or integrated into liposome membranes, we established an immunoassay protocol for specific and sensitive detection. FIG. 4 shows, that we were able to detect the receptors at concentrations as low as 75 and 100 ng/mL for GST-SytII and GT1b, respectively. In dot blot experiments, using the same antibody pairings, GST-SytII and GT1b could be detected at concentrations as low as 5 and 50 μg/mL, respectively (FIG. 6B). Experiments with complementary antibodies, i.e. coated GT1b incubated with GST antibody and coated GST-SytII incubated with GT1b antibody showed no detectable signal, which means that there was little to no cross-reactivity between the different antibody pairings.

### BoNT Binding to GST-SytII Under Acidic and Physiological Conditions

A pH-shift provides for enhanced translocation of BoNT LC via the BoNT H_{N} transmembrane channel from the cis side (i.e. endosomal lumen or medium surrounding the liposomes) to the trans side (i.e. cytosol or liposomal lumen) of a membrane [44]. Hence, it is preferred to use conditions that emulate the pH and redox gradient across endosomes, i.e. an acidic shift from pH 7.2 to 5.2, to facilitate transformation of BoNT/B H_{N} into a transmembrane channel. Hence, to elucidate, whether a pH-shift with DMG (3,3-Dimethylglutaric acid) would influence the binding affinity of BoNT/B to the respective receptors we performed the following experiment. BoNT/B was coated on a microtiter plate and GST-SytII used as antigen, which was detected by anti-GST and a respective peroxidase coupled anti-species antibody. The pH was kept at pH 7.2 for 90 min, shifted to pH 5.2 after 60 min and incubated for another 30 min, or kept at pH 5.2 for the entire 90 min during incubation of GST-SytII with the coated toxin, respectively.

FIG. 5 shows, that if pH was shifted from pH 7.2 to 5.2 even more toxin bound to coated GST-SytII than if incubated at pH 7.2 only. If binding occurred solely at pH 5.2, instead of pH 7.2, the effect was even more pronounced, and binding increased by almost 100%. This is especially interesting, as it has been proposed, that a conformational change in BoNT HC structure takes place upon an acidic pH shift [45].

Results presented in other studies, however, confirm that binding of BoNT/B to SytII at pH 5 was unaffected or even increased [46]. Although GT1b or GD1a, which is another ganglioside, SytII, and SytI are involved in BoNT/B binding [47,48], GST-SytII has in our experiments been sufficient for binding of the toxin.

Experiments performed in hippocampal neurons cultured from ganglioside KO mice showed that toxin binding was affected by absence of gangliosides [49]. Another study, however, was able to show with PC12 cells, a neuroendocrine cell line with low ganglioside contents, that SytII present in the cell membranes was sufficient to bind and mediate entry of BoNT/B into the nerve cell [50]. Accordingly, binding of BoNT/B HC_{C} to GST-SytII will remain active, so that even under acidic conditions, translocation of BoNT/B LC through the HC_{N} transmembrane channel is feasible.

### Reconstitution of Nerve Cell Receptors in Liposomal Membranes

Unfortunately, there is little information on the exact content of Glib residing in the motoneuronal membrane. Yet, a publication on the molecular composition of synaptic vesicles (SVs) gives information on lipid and protein composition and content in SV membranes [51]. The authors suggested that ceramides present approximately 0.17-1.2% and hexylceramides 8.6% of the total lipid content. In publications, where protein and lipid composition in the synaptosomal plasma membrane (SPM) were quantified, it was found that gangliosides present approximately 8.2% of all lipids [52,53]. Accordingly, we used either 0.2 to 0.5 mM GT1b in the presented experiments, which corresponds to 0.1 to 2.5% of total lipid content. Also, similar ganglioside contents (i.e. 0.11 mM) have been used for supplementation of ganglioside deficient PC12 cells tested for BoNT/B binding [50]. As for GT1b, little is known about the exact amount of SytII receptor in the presynaptic membrane. Measurements in SV, however, indicate that Sytl, a SytII analogue, constitutes approximately 7% of all SV-Proteins [51]. Yet, as GST-SytII will be the only protein receptor in the liposome membrane, there should be sufficient protein receptor molecules present to complement GT1b receptors on the liposome membranes. Although both, N-terminal domain of SytII and ganglioside are involved in BoNT/B binding [47,48], it has been reported, that truncated SytII, with N-terminus and transmembrane domain only is sufficient to bind and mediate entry of BoNT/B into nerve cells [50]. Hence, in comparison to GT1b, we used excess GST-SytII at concentrations of 0.45 to 0.72 mg/mL, i.e. 12.5 to 20 mM for production of receptor liposomes.

As GT1b integration into liposome membranes takes place via spontaneous integration of its ceramide chain during reconstitution of the lipid film, GT1b is already added to the other lipid components at the very beginning [54,55]. In case of GST-SytII, we expected that the apolar transmembrane domain of SytII [56,57] and the associated Triton X-100 molecules facilitate integration [58]. Accordingly, the protein receptor was supplemented with the aqueous medium.

Following extrusion, liposome emulsions, containing Triton X-100 were treated with SM-2 Bio-Beads to extract detergent from the liposomes. To test for integration of the receptors in the liposome membranes, we immobilised extruded receptor liposomes either on nitrocellulose membranes or on microtiter plates and analysed them with the respective receptor antibodies. FIG. 6A shows that dot blot analysis was able to detect both receptors in dialysed and undialysed receptor liposome samples and that liposomes without any receptors yielded no unspecific signal. ELISA analysis of liposomes after dialysis with 100 kDa molecular weight cut-off (MWCO) against buffer showed that if receptors were integrated into liposome membranes they could only be detected at much higher concentrations (FIG. 6B). It was found, that similar signal intensities as, e.g. for isolated GST-SytII, required 200 to 400 times higher receptor concentrations. GT1b inserted in liposome membranes yielded even lower signal, if compared to isolated GT1b.

### Detection of Nerve Cell Receptors in Liposome Fractions

To confirm and validate receptor integration into liposomes, SEC was used to separate the respective liposomes [40]. Accordingly, a refined and miniaturised SEC method that allowed reliable separation of minute volumes of liposome mixtures from not-encapsulated compounds or buffer components by hydrodynamic diameter was used. After separation, SEC fractions were analysed with the respective receptor antibodies in dot blot experiments on nitrocellulose membranes and in ELISA experiments (FIG. 7). As can be seen in FIG. 7A, GT1b and GST-SytII were detected in the first two SEC fractions. Visual control showed increased turbidity in fractions one and two as confirmation for the presence of liposomes (data not shown). Comparison of the signal obtained after SEC separation with dilutions of non-separated receptor liposomes showed that approximately 90%, and 10% of receptors could be found in the first and second fraction, respectively. ELISA detection of GST-SytII by GST antibody confirmed that the receptor elute in the liposome fractions (FIG. 7B). Despite a high limit of detection for GT1b in the ELISA immunoassay, it can be seen, that the majority of GT1b also eluted in the first SEC fractions. GT1b signal found in fractions 3 to 8 might present GT1b integrated in smaller vesicles. Because of their lower hydrodynamic diameter, they elute in later fractions and can neither be detected by visual control nor via absorption measurement at 254 nm; hence, no liposome signal is obtained in those fractions. Although the absorption spectrum of GST partly overlaps with liposome light scattering at 254 nm, visual control of the eluted fractions confirmed that liposomes eluted mainly in fractions two and three. Also, GST absorption did not seem to overlap completely with liposome detection at 254 nm, as can be seen in FIG. 7B.

Thus, visual control and detection in dot blot and ELISA experiments confirmed the presence of both GT1b and GST-SytII in liposomal fractions.

### Test for Dual Receptor Liposomes

A sandwich immunoassay in a dot blot format was used to test whether both receptor types are present on the membrane of same liposomes (FIG. 8A).

FIG. 8B shows a distinct signal when liposomes with both receptors integrated were blotted. Yet, controls with isolated receptors only also showed colouration, although the signal intensity was much lower and the spot much more diffuse. This was only observed, when isolated receptor was present that in theory can be detected by the detection antibody. As the employed antibody pairings showed neither cross reactions in this (negative control with PBS) nor in other experiments, a possible explanation might be, that due to the porous structure of the membrane, the isolated receptors did not only bind to the coated anti-receptor antibodies, but also to the membrane structure itself—in spite of blocking with PBS supplemented with 1% BSA.

Also, it has been suggested before, that GT1b and SytII co-localise in membranes due to binding events taking place between the ceramide portion of GT1b and the transmembrane domain of SytII [47]. Taken together, there is strong evidence that both receptors are present on the membrane of the same liposomes.

### VAMPtide-Assay as Reporter for BoNT/B-Proteolytic Activity

Fluorescence Characteristics of VAMPtide o-Abz Fluorophor

For measuring VAMPtide fluorescence without the necessity of the actual cleavage reaction unquenched calibration peptide of VAMPtide (uq VAMPtide; List Biological Laboratories, Inc., Campbell, Calif., USA) was used. This peptide contains the complete VAMPtide peptide sequence with a coupled o-Abz fluorophor, but without any quencher molecule.

As can be seen in FIG. 9A, emission at 418 nm (after excitation at 321 nm) displayed the highest signal intensity, although the S/N-ratio was a little below the maximum found at 410 nm. As 418 nm also represents the emission readout wavelength recommended by the manufacturer, these parameters were used for further analysis.

### VAMPtide Cleavage Under Liposome-Compatible Conditions

As the liposome lumen presents the reaction compartment for the cleavage reaction, the cleavage buffer has to be confined to the liposome interior. The cleavage buffer recommended by the manufacturer is comprised of HEPES, ZnSO₄, DTT as reducing agent and Tween-20 to increase toxin's solubilisation. Due to its size and steric hindrance, tris(2-carboyethyl)phosphine (TCEP) cannot freely diffuse through membranes. Accordingly, it has been tested as a substitute for DTT as reducing agent [45,59,60,6,61,62]. Also the detergent Tween-20 may present a problem when used within liposomes, as it may compromise the integrity of cell membranes, possibly facilitating cells lysis [63]. Also, Tween-20 may intercalate between the membrane bilayers and form mixed micelles with isolated phospholipids and membrane proteins. Accordingly, if Tween-20 was used, there might be a certain risk, that a fraction of the liposomes could be transformed from LUV into mixed micelles with lipid and detergent molecules in the membrane. Hence, attempts were made to replace DTT in the cleavage buffer by TCEP and to elucidate how the absence of Tween-20 affects VAMPtide cleavage.

First, to find the TCEP concentration, best suited for VAMPtide cleavage, the cleavage activity of 20 nM BoNT/B was tested with different TCEP concentrations, ranging from 0.75 to 5 mM, and compared with the cleavage activity in buffer supplemented with 5 mM DTT. The cleavage reaction was performed at 37° C. and fluorescence was measured with excitation and emission wavelengths of 321 nm and 418 nm, respectively. In direct comparison of the cleavage activities, 2 mM TCEP was found to be the concentration, which yielded the highest cleavage activity (FIG. 9B). Omitting Tween-20 in the cleavage buffer did not reduce the cleavage activity. Instead, cleavage activity increased considerably (FIG. 9C). Hence, replacing 5 mM DTT by 2 mM TCEP and omitting Tween-20 in the cleavage buffer did not affect VAMPtide cleavage considerably.

Similar observations have been made in other studies, where 1 and 2 mM TCEP were found to be the best concentrations for VAMPtide cleavage by BoNT/B and BoNT/B LC, respectively [64,65]. In other studies that employed VAMPtide for measuring BoNT/B LC cleavage, Tween-20 has always been supplemented in the cleavage buffer. The manufacturer of VAMPtide states that in case of SNAPtide, the analogous reporter peptide for BoNT/A LC, cleavage was also measured in the absence of Tween-20, though at lower levels. The manufacturer hypothesises that Tween-20 may help to disperse non-specifically bound enzyme or substrate from the walls of the microtiter well, so that more enzyme and substrate are available for interaction [66]. So, apparently specificity of VAMPtide cleavage by BoNT/B LC should not be impaired by the absence of Tween-20 in the cleavage buffer. As our results confirm good cleavage activity even without Tween-20, the described buffer composition without Tween-20 and with 2 mM TCEP in 20 mM HEPES (pH 7.4) supplemented with 0.05 mM ZnSO₄ is preferred.

### VAMPtide Cleavage by Different Toxin Preparations

Apart from BoNT/B, we tested the cleavage activity of isolated BoNT/B LC, TeNT, LH_{N}B, and scLH_{N}B in the modified cleavage buffer. Early experiments showed that isolated BoNT/B LC did not exhibit any cleavage activity under these conditions. Instead, supplementation of 0.2% Tween-20 restored most of the cleavage activity of isolated BoNT/B LC, while DTT supplementation restored the function only partly (data not shown). As expected, the cleavage reaction did neither work for TeNT, as VAMPtide did not contain the specific recognition site for TeNT LC. In contrast to isolated BoNT/B LC and TeNT, BoNT/B and BoNT/B complex displayed typical cleavage activity, with a limit of detection of 2 and 5 nM, respectively (FIG. 9D & Table 3).

TABLE 3
VAMPtide cleavage activities by different BoNT/B derivatives. Cleavage by BoNT/B
derivates (concentrations 1-20 nM) under different buffer conditions (values represent
Vₘₐₓ s⁻¹ from t = 15-60 min) measured with the VAMPtide assay.

| Toxin | | | | | | | | | | | |
|---|---|---|---|---|---|---|---|---|---|---|---|
| derivate | Buffer type | 1 nM | SD | 2 nM | SD | 5 nM | SD | 10 nM | SD | 20 nM | SD |
| BoNT/B | +TCEP | 0.027 | 0.021 | 0.016 | — | 0.045 | 0.016 | 0.079 | 0.024 | 0.126 | 0.062 |
| | +TCEP+Tween | | | 0.021 | — | 0.045 | — | 0.076 | — | 0.111 | 0.052 |
| | +DTT+Tween | | | | | | | 0.044 | — | 0.055 | 0.006 |
| LHnB | +TCEP | 0.041 | — | | | 0.051 | 0.000 | 0.072 | 0.007 | 0.110 | 0.051 |
| | +TCEP+Tween | | | | | | | | | | |
| | +DTT+Tween | | | | | | | | | | |
| scLHnB | +TCEP | 0.044 | — | | | 0.063 | 0.006 | 0.084 | 0.027 | 0.143 | 0.073 |
| | +TCEP+Tween | | | | | | | | | | |
| | +DTT+Tween | | | | | | | | | | |
| BoNT/B LC | +TCEP | | | | | | | | | | |
| | +TCEP+Tween | | | | | | | | | | |
| | +DTT+Tween | | | | | | | 0.198 | 0.017 | 0.359 | 0.037 |
| BoNT/B | +TCEP | | 0.014 | — | 0.032 | 0.006 | 0.074 | — | 0.097 | — | |
| complex | +TCEP+Tween | | | | | | | | | | |
| | +DTT+Tween | | | | | | | | | | |

LH_{N}Band scLH_{N}B are BoNT/B truncation mutants devoid of the receptor-binding domain. Also, scLH_{N}B is still connected to the BoNT/B LC not only via disulphide, but also via a peptide bond. As the LC in these toxin derivatives is still active, VAMPtide could be successfully cleaved under the modified buffer conditions, both with detection limits of 1 nM. Although the actual enzymatic cleavage activities (Vₘₐₓ s⁻¹) showed very high inter-assay variations, it can be said, that the modified cleavage buffer conditions allows cleavage of VAMPtide by BoNT/B, LH_{N}B and scLH_{N}B (FIGS. 9E&F).

Other groups that used VAMPtide molecule as reporter for BoNT/B LC endopeptidase activity found similar results, e.g. detection of 0.25, 6 and 10 nM BoNT/B LC in buffer after incubation with 10, 4.2 and 20 μM VAMPtide, respectively [67,68,64,65,69]. For BoNT/B, detection was possible for 3.3 to 6.7 μg mL⁻¹, i.e. 22 to 44 nM [64,65]. The values obtained are still above the values that could theoretically be achieved. The manufacturer described detection limits as low as 0.2 nM for BoNT/B, and even 78 μM for BoNT/B LC, using similar machinery and settings [70]. Results for BoNT/B LC, however, were only obtained when endpoint readings were obtained after 24 hours incubation at 37° C. Using magnetic bead enrichment prior to the VAMPtide cleavage assay, even allowed detection at concentrations as low as 10 ng mL⁻¹, i.e. 0.07 nM BoNT/B [67]. Concerning the dependence of BoNT/B LC cleavage activity on Tween-20 and/or DTT, another group made similar observations [69]. They found, that nonreduced LC exists as a homodimer, which reduces the flexibility of LC molecules to move around and to coordinate with the substrate for the enzymatic reaction to occur. High cleavage activities have been found in DTT containing cleavage buffer (Table 2). Interestingly, if 2 mM TCEP is used instead of 5 mM DTT, LC displays almost no cleavage activity. In the complete neurotoxin, consisting of heavy chain (HC) and light chain (LC), however, part of the HC belt hangs above the open pocket of the LC in which the active site is located, so that latter becomes more difficult for the substrate to access, thus showing a lower level of activity than the LC in buffer supplemented with DTT [69]. In case of LHnB and scLHnB, which are devoid of the BoNT/B HC binding domain, less steric hindrance is present, thus allowing for higher cleavage activities. Here, however, TCEP seems to allow for sufficient reduction. This might, to a large part, be due to the fact that the two toxin derivatives resemble structure-wise more to the holotoxin more than the LC. BoNT/B complex showed similar, but slightly lower cleavage activities than the holotoxin if DTT was present. This exemplary assay provides sensitivity for BoNT/B cleavage activity at concentrations as low as 2-5 nM.

Incorporation of VAMPtide Reporter Peptide into the Assay Liposomes

VAMPtide molecules are incorporated into liposomes by adding them together with the aqueous medium for lipid film reconstitution. Upon forming of MLV, VAMPtide molecules become enclosed by liposomal membranes. With subsequent extrusion through PC membranes, a distinct portion of the molecules gets encapsulated, while another part stays outside the liposomes. To test for the actual encapsulation rate of VAMPtide molecules into the liposomes, without the need for any cleavage reaction, we used a VAMP antibody and corresponding peroxidase coupled anti-species antibody for detection.

In FIG. 10A, sensitivity is shown for VAMPtide coated in concentrations ranging from 5 to 100 ng/mL. The assay showed excellent sensitivity with a detection limit as low as 5 ng/mL. In dot blot experiments VAMP antibody detected VAMPtide at concentrations as low as 2.5 μM, while not detecting unquenched VAMPtide calibration peptide (100 μM), unquenched SUQ-FITC (100 μM), GT1b (50 μg/mL) or empty liposomes (data not shown). Unfortunately, anti-VAMP antibody recognized GST-SytII at a concentration of 7.2 mg/mL. However, as GST-SytII molecules will be much less concentrated in the final liposomes, this will not affect immunological detection of VAMPtide molecules in presence of the final receptor liposomes. To test for the actual encapsulation efficiency of VAMPtide, liposomes, supplemented with 200 and 100 μM VAMPtide in the aqueous reconstitution medium, were separated after extrusion via SEC from not-encapsulated VAMPtide molecules. Analysis of the collected SEC fractions, after liposome lysis in 3% Triton X-100 in an ELISA immunoassay, with anti-VAMP antibody showed that VAMPtide eluted mainly in fractions two and three (FIG. 10B), correlating with the liposome elution peak observed by visual control. In parallel, a standard curve for VAMPtide content in non-separated liposome samples was generated and the summed signals of fractions two and three were used to calculate for the respective encapsulation efficiency EE[%].

TABLE 4
Encapsulation efficiencies of different VAMPtide concentrations
into LUV.

VAMPtide
concentration
[mM]

| | 200 μM | 100 μM |
|---|---|---|
| Encapsulation efficiency EE [%] | 73.07 | 66.61 |
| Standard deviation (SD) | +/−23.14 | +/−16.19 |

As shown in Table 4, EE[%] values were very high, with approximately 73 and 67% of all VAMPtide molecules encapsulated into liposomes, i.e. 146.14+/−46.28 μM (original 200 μM) or 66.61+/−16.19 μM (original 100 μM) VAMPtide encapsulated. High Standard Deviations (SD) were due to the narrow range of linear detection in the ELISA immunoassay (FIGS. 10C&D). It is evident, that if a lower original VAMPtide concentration was used, the encapsulation efficiency is also negatively affected. The percentage of encapsulated VAMPtide corresponds to 14.6+/−4.6 times and 6.7+/−1.6 times of the concentration recommended by the manufacturer (10 μM) for sensitive detection of BoNT/B cleavage activity. Hence, even in diluted VAMPtide liposomes, sufficient peptide reporter would be present to allow detection of the cleavage activity. In another experiment, it has been found, that fluorescence readout of unquenched calibration peptide of VAMPtide encapsulated into liposomes was slightly affected by high liposome concentration (data not shown). Accordingly, high encapsulation efficiencies would later allow for dilution of the liposomes, thus reducing the interference with the fluorescence measurement. In general, it can be seen, that simple extrusion encapsulates a high percentage of VAMPtide molecules into liposomes.

### Test for Non-Specific Membrane Binding of VAMPtide

DNP (2,4-dinitrophenol) and o-Abz (o-Aminobenzoic acid) are coupled to the VAMPtide molecule as FRET acceptor and donor fluorophor, respectively. It is known, that DNP exhibits hydrophobic properties [71] and o-Abz may also dissolve in nonpolar solvents [72]. Hence, instead of being properly encapsulated, it might be that VAMPtide is only loosely associated to the liposomes and/or integrated only by part into the liposome membranes [73]. This could influence the calculated encapsulation efficiency, as BoNT/B could cleave the associated VAMPtide molecules without any binding or translocation needed. Accordingly, we intended to rule this out, by testing for not-encapsulated VAMPtide with a protease protection assay. In this assay, VAMPtide molecules, which are only loosely associated to the liposomes, and VAMPtide molecules that are partly integrated into the membrane, but whose peptide chains extend into the liposome exterior, would be cleaved by Proteinase K (Sigma-Aldrich, Buchs, Switzerland). Proteinase K is a broadspectrum serine protease, which predominantly cleaves the peptide bond adjacent to the carboxyl group of aliphatic and aromatic amino acids with blocked alpha amino groups [74].

First, to test whether Proteinase K is able to cleave VAMPtide molecule, we subjected a solution of 200 μM VAMPtide to digestion with 100 μg/mL of Proteinase K. Digestion was performed for 1 h in a 37° C. waterbath. FIG. 11A shows that Proteinase K successfully cleaved VAMPtide. While signal for VAMPtide in the immunoassay decreased upon cleavage, the fluorescence signal increased as cleavage reduced the quenching effect.

This means, that Proteinase K was able to cleave VAMPtide. Subsequently, VAMPtide liposomes from the previous experiment with approximately 146.14+/−46.28 μM VAMPtide encapsulated (original 200 μM) were subjected to digestion for 60 min at 37° C. with 50 and 100 μg/mL of Proteinase K, respectively. After digestion, VAMPtide liposomes were fractioned by SEC, and measured with VAMP antibody in an ELISA assay (FIG. 11B). Prior to coating on microtiter plates for ELISA analysis, Triton X-100 was added at a concentration of 3% to lyse liposomes and to guarantee detection of encapsulated and not-encapsulated VAMPtide. As coating took place at 4° C., no more VAMPtide cleavage took place at this point, even if Proteinase K was present. The immunoassay results showed that signal intensities in the liposome fractions two and three in samples digested with Proteinase K were only little lower than those, which were found in undigested samples. These results suggest that VAMPtide molecules are either thoroughly encapsulated into the liposomes and/or that they may be integrated into the liposome membrane in whichever modality and cannot be cleaved by an endopeptidase such as Proteinase K, even at concentrations as high as 100 μg/mL.

### EXAMPLE 3

### Characterization of Cleavage Substrates for BoNT/A and BoNT/B

### 3.1 Assay Format

Cleavage assays were performed in black 96 half-well microplates (Greiner Bio-One GmbH, Frickenhausen, Germany) with a reaction volume 100 μL.
For the reaction buffer (composition corresponding to the respective substrate; see below), only components (HEPES, ZnCl₂, ZnSO₄, TCEP (with an adjusted pH to 7.0), DTT, and ultrapure H₂O) of molecular biology grade reagents are used.

In the actual assay, the respective substrate is mixed with 10 μL of the corresponding 10 times concentrated reaction buffer (RB) and the toxin or respective sample. Finally, H₂O (ultrapure; 37° C.) is added to give a final volume of 100 μL (in black 96 half-well microplates pre-warmed to 37° C.). The measurement is then performed with a SpectraMax GeminiXS or any other spectro-fluorometry microplate reader at 37° C. assay temperature. For signal readout the samples are excited at the excitation and emission wavelength combination of the respective substrate (see below).

### 3.2 Pharmaleads Cleavage Substrates

### 3.2.1 Description & Experimental Setup

### 3.2.1.1 BoNT/A-Substrate

PL50 (Pharmaleads, Paris, France) is a peptide, which is intramolecularly quenched by fluorescence resonance energy transfer (FRET). Pyrenylalanine (Pya) is the fluorophore Para-nitro-phenylalanine (Nop) the acceptor chromophore. PL50 contains a specific cleavage site for BoNT/A LC [83,84]. Lyophilised PL50 stock is dissolved in 80:20 ultrapure H₂O and DMF (N,N-Dimethylformamide) and for experiments with PL50 the following reaction buffer (RB_{PL150}) is used: 10 mM HEPES (pH 6.2), 75 μM ZnSO₄, 2.5 mM TCEP (pH 7.0). For cleavage experiments, PL50 is used at an assay concentration of 10 μM. Upon cleavage of the substrate by BoNT/A, the fluorescence can be measured with an excitation wavelength of λex=341 nm and an emission wavelength of λem=397 nm.
The sequence information of PL50 is as follows: [(Ac-156-203) SNAP-25](Nop197, Pya200, Nle202):
¹⁵⁶AcIIGNLRHMALDMGNEIDTQNRQIDRIMEKADSNKTRIDEAN-Nop-RA-Pya-KNIeLNH₂²⁰³

### 3.2.2.2 BoNT/B-Substrate

PL150 (Pharmaleads, Paris, France) is a peptide, which is intramolecularly quenched by fluorescence resonance energy transfer (FRET). Pyrenylalanin (Pya) is the fluorophore Para-nitro-phenylalanin (Nop) the acceptor chromophore. PL150 contains a specific cleavage site for BoNT/B LC [85,86,87]. Lyophilised PL150 stock is dissolved in ultrapure H₂O and for experiments with PL150 the following reaction buffer (RB_{PL150}) is used: 10 mM HEPES (pH 7.2), 75 μM ZnSO₄, 2.5 mM TCEP (pH 7.0). For cleavage experiments, PL150 is used at an assay concentration of 10 μM. Upon cleavage of the substrate by BoNT/B, the fluorescence can be measured with an excitation wavelength of λex=341 nm and an emission wavelength of λem=397 nm.
The sequence information of PL150 is as follows: [(Ac-60-94)VAMP](Pya74,Nop77): ⁶⁰AcLSELDDRADALQAG-Pya-SQ-Nop-ESSAAKLKRKYWWKNLKNH₂⁹⁴

### 3.2.2 Results

As can be seen in FIG. 12, at least 10 pM of BoNT/A or BoNT/B could be detected with the PL50 or PL150 substrate, respectively. Furthermore, detection of PL50 cleavage was also possible in the presence of 10, 5 or 0% (v/v) empty liposomes (FIG. 13), although higher liposome concentrations lead to a slower signal increase (Vmax per min).

To allow for later experiments to detect PL50 and PL150 e.g. in liposome fractions or for encapsulation studies, immunological detection was tested in ELISA experiments. Therefore, different concentrations of either substrate were coated over night at 4° C. on microplates (clear MaxiSorp microplates; Nunc, Langenselbold, Germany). Bound PL50 and PL150 were detected with polyclonal rabbit@SNAP25 antiserum or affinity purified rabbit@VAMP1,2,3, respectively (SynapticSystems GmbH, Goettingen, Germany). The antibodies themselves were subsequently detected with goat@rabbit-HRP (BioFX Laboratories Inc., Owings Mills Md., USA) and TMB (Pierce Biotechnology Inc., Rockford Ill., USA) as substrate. As can be seen in FIG. 14, at least 40 nM of PL50 and at least 50 nM of PL150 were detectable using the described ELISA setup.

### 3.3 List Biological Laboratories Cleavage Substrates

### 3.3.1 Description & Experimental Setup

### 3.3.1.1 BoNT/A Substrates

SNAPtide_{IAF} (SNAPtide (DABCYL/5-IAF); List Biological Laboratories Inc., Campbell, Calif., USA) is a peptide, which is labelled using 5-Iodoacetamido-fluorescein to obtain an S-fluoresceinyl cysteine fluorophore on the C-terminal. The acceptor chromophore is DABCYL [88]. Lyophilised SNAPtide_{IAF} stock is dissolved in ultrapure H₂O and for experiments with SNAPtidep_{IAF}, the following reaction buffer (RB_{SNAPtide-IAF}) is used: 20 mM HEPES (pH 7.5), 15 mM ZnCl₂, 1.25 mM DTT, 0.1% Tween-20. For cleavage experiments, SNAPtide_{IAF} is used at an assay concentration of 10 μM. Upon cleavage of the substrate by BoNT/A, the fluorescence can be measured with an excitation wavelength of λex=490 nm and an emission wavelength of λem=523 nm with a cutoff filter set at 495 nm.

SNAPtide_{FITC} (SNAPtide (FITC/DABCYL); List Biological Laboratories Inc., Campbell, Calif., USA) is a peptide, which is intramolecularly quenched by fluorescence resonance energy transfer (FRET). The N-terminally-linked fluorophore is fluorescein-thiocarbamoyl (FITC) and the acceptor chromophore is DABCYL [89]. Lyophilised SNAPtide_{FITC} stock is dissolved in ultrapure H₂O and for experiments with SNAPtide_{FITC}, the following reaction buffer (RB_{SNAPtide-FITC}) is used: 20 mM HEPES (pH 7.5), 15 mM ZnCl₂, 1.25 mM DTT, 0.1% Tween-20. Upon cleavage of the substrate by BoNT/A, the fluorescence can be measured with an excitation wavelength of λex=490 nm and an emission wavelength of λem=523 nm with a cutoff filter set at 495 nm.

SNAPtide_{o-Abz} (SNAPtide (o-Abz/Dnp); List Biological Laboratories Inc., Campbell, Calif., USA) is a peptide, which is intramolecularly quenched by fluorescence resonance energy transfer (FRET). The N-terminally-linked fluorophore is o-aminobenzoic acid (o-Abz) and the acceptor chromophore is a 2,4-dinitrophenyl group (Dnp) [89,90,91]. Lyophilised SNAPtide_{FITC} stock is dissolved in ultrapure H₂O and for experiments with SNAPtide_{o-Abz}, the following reaction buffer (RB_{SNAPtide-o-Abz}) is used: 20 mM HEPES (pH 8.0), 0.75 mM ZnSO₄, 1.25 mM DTT. Upon cleavage of the substrate by BoNT/A, the fluorescence can be measured with an excitation wavelength of λex=320 nm and an emission wavelength of λem=421 nm.

### 3.3.1.2 BoNT/B Substrate

VAMPtide_{o-Abz} (VAMPtide (o-Abz/Dnp); List Biological Laboratories Inc., Campbell, Calif., USA) is a peptide, which is intramolecularly quenched by fluorescence resonance energy transfer (FRET). The N-terminally-linked fluorophore is o-aminobenzoic acid (o-Abz) and the acceptor chromophore is a 2,4-dinitrophenyl group (Dnp). Lyophilised VAMPtide_{o-Abz} stock is dissolved in ultrapure H₂O and for experiments with VAMPtide_{o-Abz}, the following reaction buffer (RB_{VAMPtide-o-Abz}) is used: 10 mM HEPES (pH 7.2), 0.075 mM ZnSO₄, 2.5 mM TCEP (pH 7.0). Upon cleavage of the substrate by BoNT/B, the fluorescence can be measured with an excitation wavelength of λex=320 nm and an emission wavelength of λem=421 nm.

### 3.3.2 Results

As can be seen in FIG. 15, List Biological Laboratories substrates allowed detection of at least either 1 nM BoNT/A (FIG. 4A-C) or 2 nM BoNT/B (FIG. 15D). Furthermore, SNAPtide_{FITC} and SNAPtide_{o-Abz} even allowed detection of 0.5 nM BoNT/A.
To allow for later experiments to detect VAMPtide_{o-Abz} e.g. in liposome fractions or for encapsulation studies, immunological detection was tested in ELISA experiments. Therefore, different concentrations of either substrate were coated over night at 4° C. on microplates (clear MaxiSorp microplates; Nunc, Langenselbold, Germany). Bound VAMPtide_{o-Abz} ewas detected with affinity purified rabbit@VAMP1,2,3, (SynapticSystems GmbH, Goettingen, Germany). The antibodyy was subsequently detected with goat@rabbit-HRP (BioFX Laboratories Inc., Owings Mills Md., USA) and TMB (Pierce Biotechnology Inc., Rockford Ill., USA) as substrate. As can be seen in FIG. 14B, at least 50 nM of VAMPtide_{o-Abz} could be detected using the described ELISA setup.

### EXAMPLE 4

### Production of Liposomes with Receptors Specific for BoNT/A (A-Liposomes)

### 4.1 Description of Specific Receptors

For production of A-liposomes (liposomes with the receptors, which are bound by BoNT/A), SV2c represents a suitable protein receptor [92,93]. Therefore, truncated and modified GST-SV2c (kindly provided by A. Rummel, M H Hannover, Hannover, Germany) was used. The SV2c used for GST-SV2c, was derived from human and contains the complete luminal domain (aa454-579) plus a C-terminal transmembrane domain (aa580-603) and a N-terminally attached GST-tag. GT1b, just as for BoNT/B, also serves as ganglioside receptor for BoNT/A (Matreya, Pleasant Gap, Pa., USA).

### 4.2 Description of A-Liposome Production

As for production of B-liposomes, the lipid receptor (ganglioside GT1b) was evaporated together with the used lipids (40 mM DOPC/Cholesterol; same as for B-liposomes) from organic solution to form a lipid film; GT1b was used at the same concentrations described for B-liposomes. The protein receptor for BoNT/A (GST-SV2c) was applied to the lipid film together with the future reaction buffer (RB_{PL50}); GST-SV2c was used at 12.5 or 8 μg/mL (final concentration in the liposome emulsion). Except for the receptors used, the production process for A-liposomes is the same as for B-liposomes, including removal of Triton X-100 by treatment with BioBeads SM-2.

### 4.3 Results

After production, the A-liposomes were analysed via dynamic light scattering and were found to have an average diameter of approximately 190 nm. After separation of the A-liposomes by size exclusion chromatography the derived fractions were analysed regarding the contained receptors. Detection of receptors (GST-SV2c & GT1b) in A-Liposome fractions after SEC was performed on ELISA microplates. Samples were diluted 1/100 and coated at 4° C. over night. As can be seen in FIG. 16, both receptors can be found in the same fractions as the liposomes. As can be seen in FIG. 16, the majority of receptor molecules could be found in the same fractions as the liposomes. Accordingly, we were able to produce A-liposomes with receptors specific for binding of BoNT/A.

### EXAMPLE 5

### Production of FAL (Fully Assembled Liposomes)

### 5.1 Description of FAL Production

The receptors, i.e. protein receptor GST-SytII for B-FAL and GST-SV2c for A-FAL, respectively, and lipid receptor GT1b for both FAL, are used at the same concentration as for production of B- and A-liposomes, respectively. In the first step, GT1b is evaporated from organic solvent (Methanol/Dichloromethane 1:1) together with the lipids (40 mM DOPC/Cholesterol). For production of FAL, the resulting lipid film is hydrated in a mixture of GST-SytII or GST-SV2c, the respective substrate, i.e. SNAPtide or PL50 for A-FAL or VAMPtide or PL150 for B-FAL, respectively, and the corresponding reaction buffer. Cleavage substrates are used at a concentration of 200 μM. The resulting emulsion, consisting of multilamellar and multivesicular vesicles of different sizes, is subsequently extruded through with a MiniExtruder (Avanti Polar Lipids, Alabaster, Ala., USA) through track-etched polycarbonate membranes with pore diameters ranging from 50-400 nm. The protein receptors, GST-SytII or GST-SV2c, respectively, were previously isolated from cellular membranes via the creation of Triton X-100 micelles. To circumvent any leakage from the newly created FAL, the remaining Triton X-100 is removed from the preparation with BioBeads SM-2 (Bio-Rad, Reinach, Switzerland), according to the manufacturer's instructions. Subsequently, to remove any not-encapsulated substrate molecules and to remove the components of the reaction buffer from the surrounding medium, FAL are dialysed with a Spectra/Por7 regenerated cellulose membrane (MWCO=25,000 Dalton; Spectrum Labs, Breda, Netherlands) against HEPES buffer of the appropriate molarity.
   5.2 Release of Cleavage Substrate from A-FAL and B-FAL
A-FAL_{PL50} and B-FAL_{PL150} were tested with this method to elucidate whether the substrate molecules are really encapsulated into the liposomes and do not reside in the membrane or adhere to the liposome surface. Both FAL types were also diluted 1/20 and treated with either 100 μM BoNT/A or BoNT/B in the respective RB. If the substrate only resides in the liposome lumen, then addition of 0.1% Triton X-100 should cause lysis of the liposomes and release of the substrate, which can subsequently be cleaved by BoNT/B in the surrounding medium. As can be seen by the sharp increase in fluorescence following the addition of Triton X-100 (FIG. 17), the liposome lysis causes release of the substrate molecules, which are then cleaved by the BoNT molecules in the surrounding medium. Accordingly, the majority of substrate molecules contained in A- and B-FAL are located in the liposome lumen.

### 5.3 Cleavage Substrates in B-FAL

SEC separation of B-FAL_{PL150} and B-FAL_{VAMPtide-o-Abz}, respectively, shows that the majority of PL150 and VAMPtide_{o-Abz} molecules elute in the liposome fractions (FIG. 18). For detection of the substrate molecules the samples were diluted 1/2000 in 10 mM HEPES buffer with 0.1% Triton X-100 and coated on ELISA microplates at 4° C. over night.

### REFERENCES

1. Böhnel H, Gessler F (2005) Botulinum toxins—cause of botulism and systemic diseases? Vet Res Commun 29 (4):313-345.
2. CDC (1998) Botulism in the United States, 1899-1996. Handbook for Epidemiologists, Clinicians, and Laboratory Workers, vol Centers for Disease Control and Prevention. Atlanta, Ga., USA.
3. Binz T, Rummel A (2009) Cell entry strategy of clostridial neurotoxins. J Neurochem 109 (6):1584-1595.
4. Brunger A T, Rummel A (2009) Receptor and substrate interactions of clostridial neurotoxins. Toxicon 54 (5):550-560.
5. Couesnon A, Shimizu T, Popoff M R (2009) Differential entry of botulinum neurotoxin A into neuronal and intestinal cells. Cell Microbiol 11 (2):289-308.
6. Montal M (2009) Translocation of botulinum neurotoxin light chain protease by the heavy chain protein-conducting channel. Toxicon 54 (5):565-569.
7. Chen S, Barbieri J T (2006) Unique substrate recognition by botulinum neurotoxins serotypes A and E. J Biol Chem 281 (16)10906-10911.
8. Schiavo G, Rossetto O, Benfenati F, Poulain B, Montecucco C (1994) Tetanus and botulinum neurotoxins are zinc proteases specific for components of the neuroexocytosis apparatus. Ann New York Acad Sci 710:65-75.
9. Sikorra S, Henke T, Galli T, Binz T (2008) Substrate recognition mechanism of VAMP/synaptobrevin-cleaving clostridial neurotoxins. J Biol Chem 283 (30):21145-21152.
10. Amon S, Schechter R, Inglesby T, Henderson D, Bartlett J, Ascher M, Eitzen E, Fine A, Hauer J, Layton M, Lillibridge S, Osterholm M, O'Toole T, Parker G, Perl T, Russell P, Swerdlow D, Tonat K (2001) Botulinum toxin as a biological weapon: medical and public health management. JAMA 285 (8):1059-1070.
11. Herrero B, Ecklung A, Streett C, Ford D, King J (1967) Experimental botulism in monkeys—a clinical pathological study. Exp Mol Pathol 6 (1):84-95.
12. Lund B (1990) Foodborne disease due to Bacillus and Clostridium species. Lancet 336 (8721):982-986.
13. Kerner J (1817) Vergiftung durch verdorbene Würste. Tübinger Blätter für Naturwissenschaften and Arzneyenkunde 3 (1):1-25.
14. Shukla H D, Sharma S K (2005) Clostridium botulinum: a bug with beauty and weapon. Crit Rev Microbiol 31 (1):11-18.
15. Cherington M (1998) Clinical spectrum of botulism. Muscle Nerve 21 (6):701-710.
16. Jankovic J (2004) Botulinum toxin in clinical practice. J Neurol Neurosurg Psychiatr 75 (7):951-957.
17. Schantz E J, Kautter D A (1978) Microbiological methods: Standardized assay for Clostridium botulinum toxins. J AOAC 6:96.
18. Balls M (2003) Botulinum toxin testing in animals: the questions remain unanswered. ATLA 31 (6):611-615.
19. Bottrill K (2003) Growing old disgracefully: the cosmetic use of botulinum toxin. ATLA 31 (4):381-391.
20. EDQM (2011) 07/1022:2113 Botulinum toxin type A for injection. In: European Pharmacopoeia 7.2. pp 3613-3615.
21. Scotcher M C, Cheng L W, Stanker L H (2010) Detection of Botulinum Neurotoxin Serotype B at Sub Mouse LD50 Levels by a Sandwich Immunoassay and Its Application to Toxin Detection in Milk. PLoS One 5 (6):e11047(11001-11010).
22. Ahn-Yoon S, DeCory T R, Durst R A (2004) Ganglioside-liposome immunoassay for the detection of botulinum toxin. Anal Bioanal Chem 378:68-75.
23. Adler S, Bicker G, Bigalke H, Bishop C, Blumel J, Dressler D, Fitzgerald J, Gessler F, Heuschen H, Kegel B, Luch A, Milne C, Pickett A, Ratsch H, Ruhdel I, Sesardic D, Stephens M, Stiens G, Thornton P, Thurmer R, Vey M, Spielmann H, Grune B, Liebsch M (2010) The current scientific and legal status of alternative methods to the LD50 test for botulinum neurotoxin potency testing. The report and recommendations of a ZEBET Expert Meeting. ATLA 38 (4):315-330.
24. Hines H B, Kim A D, Stafford R G, Badie S S, Brueggeman E E, Newman D J, Schmidt J J (2008) Use of a recombinant fluorescent substrate with cleavage sites for all botulinum neurotoxins in high-throughput screening of natural product extracts for inhibitors of serotypes A, B, and E. Appl Environ Microbiol 74 (3):653-659.
25. Mason J T, Xu L, Sheng Z-m, O'Leary T J (2006) A liposome-PCR assay for the ultrasensitive detection of biological toxins. Nat Biotechnol 24 (5):555-557.
26. Dong M, Tepp W, Johnson E, Chapman E (2004) Using fluorescent sensors to detect botulinum neurotoxin activity in vitro and in living cells. Proc Natl Acad Sci USA 101 (41):14701-14706.
27. Pellett S, Tepp W, Clancy C, Borodic G, Johnson E (2007) A neuronal cell-based botulinum neurotoxin assay for highly sensitive and specific detection of neutralizing serum antibodies. FEBS Lett 581 (25):4803-4808.
28. Pellett S, Du Z, Pier C L, Tepp W H, Zhang S, Johnson E A (2011) Sensitive and quantitative detection of botulinum neurotoxin in neurons derived from mouse embryonic stem cells. Biochem Biophys Res Commun 404 (1):388-392.
29. Wang H-H, Riding S, Lindo P, Singh B R (2010) Endopeptidase Activities of Botulinum Neurotoxin Type B Complex, Holotoxin, and Light Chain. Appl Environ Microbiol 76 (19):6658-6663.
30. Fischer A, Nakai Y, Eubanks L M, Clancy C M, Tepp W H, Pellett S, Dickerson T J, Johnson E A, Janda K D A, Montal M (2009) Bimodal modulation of the botulinum neurotoxin proteinconducting channel. Proc Natl Acad Sci USA 106 (5):1330-1335.
31. Adler S, Bicker G, Bigalke H, Bishop C, Blumel J, Dressler D, Fitzgerald J, Gessler F, Heuschen H, Kegel B, Luch A, Milne C, Pickett A, Ratsch H, Ruhdel I, Sesardic D, Stephens M, Stiens G, Thornton P D, Thurmer R, Vey M, Spielmann H, Grune B, Liebsch M (2010) The current scientific and legal status of alternative methods to the LD50 test for botulinum neurotoxin potency testing. The report and recommendations of a ZEBET Expert Meeting. Altern Lab Anim 38 (4):315-330.
32. Mahrhold S, Rummel A, Bigalke H, Davletov B, Binz T (2006) The synaptic vesicle protein 2C mediates the uptake of botulinum neurotoxin A into phrenic nerves. FEBS Lett 580:2011-2014.
33. Pickett A, O'Keeffe R, Judge A, Dodd S (2008) The in vivo rat muscle force model is a reliable and clinically relevant test of consistency among botulinum toxin preparations. Toxicon 52 (3):455-464. doi:10.1016/j.toxicon.2008.06.021.
34. Schantz E J, Kautter D A (1978) Microbiological methods: Standardized assay for Clostridium botulinum toxins. J AOAC 6:96.
35. Zepik H H, Walde P, Kostoryz E L, Code J, Yourtee D M (2008) Lipid vesicles as membrane models for toxicological assessment of xenobiotics. Crit Rev Toxicol 38 (1):1-11.
36. Wang X, Quinn P J (1999) Vitamin E and its function in membranes. Progr Lipid Res 38 (4):309-336.
37. Hug P, Sleight R G (1991) Liposomes for the transformation of eukaryotic cells. Biochim Biophys Acta 1097 (1):1-17.
38. Katsel P L, Greenstein R J (2000) Eukaryotic gene transfer with liposomes: effect of differences in lipid structure. Biotechnol Annu Rev 5:197-220.
39. Lohner K, Sevcsik E, Pabst G (2008) Liposome-Based Biomembrane Mimetic Systems: Implications for Lipid-Peptide. In: Tien H, Ottova-Leitmannova A (eds) Advances in Planar Lipid Bilayers and Liposomes, vol 6. pp 103-137.
40. Zuidam N J, Vrueh Rd, Crommelin D J A (2003) Characterization of Liposomes. In: Torchilin V P, Weissig V (eds) Liposomes: A Practical Approach. The Practical Approach Series, 2 edn. Oxford University Press, Oxford, USA, pp 31-72.
41. Niggemann G, Kummrow M, Helfrich W (1995) The Bending Rigidity of Phosphatidylcholine Bilayers Dependences on Experimental Method, Sample Cell Sealing and Temperature. J Phys II 5 (3):413-425.
42. Pan J, Tristram-Nagle S, Kucerka N, Nagle J F (2008) Temperature dependence of structure, bending rigidity, and bilayer interactions of dioleoylphosphatidylcholine bilayers. Biophys J 94 (1):117-124.
43. Rawicz W, Olbrich K C, McIntosh T, Needham D, Evans E (2000) Effect of chain length and unsaturation on elasticity of lipid bilayers. Biophys J 79 (1):328-339.
44. Brunger A T, Breidenbach M A, Jin R, Fischer A, Santos J S, Montal M (2007) Botulinum neurotoxin heavy chain belt as an intramolecular chaperone for the light chain. PLoS Pathog 3 (9):1191-1194.
45. Fischer A, Montal M (2007) Crucial role of the disulfide bridge between botulinum neurotoxin light and heavy chains in protease translocation across membranes. J Biol Chem 282 (40):29604-29611.
46. Brandau D T, Joshi S B, Smalter A M, Kim S, Steadman B, Middaugh C R (2007) Stability of the Clostridium botulinum type A neurotoxin complex: an empirical phase diagram based approach. Mol Pharm 4 (4):571-582.
47. Kozaki S, Kamata Y, Watarai S, Nishiki T, Mochida S (1998) Ganglioside GT1b as a complementary receptor component for Clostridium botulinum neurotoxins. Microb Pathog 25 (2):91-99.
48. Nishiki T, Tokuyama Y, Kamata Y, Nemoto Y, Yoshida A, Sato K, Sekiguchi M, Takahashi M, Kozaki S (1996) The high-affinity binding of Clostridium botulinum type B neurotoxin to synaptotagmin II associated with gangliosides GT1b/GD1a. FEBS Lett 378 (3):253-257.
49. Dong M, Tepp W H, Liu H, Johnson E A, Chapman E R (2007) Mechanism of botulinum neurotoxin B and G entry into hippocampal neurons. J Cell Biol 179 (7):1511-1522.
50. Dong M, Richards D A, Goodnough M C, Tepp W H, Johnson E A, Chapman E R (2003) Synaptotagmins I and II mediate entry of botulinum neurotoxin B into cells. J Cell Biol 162 (7):1293-1303.
51. Takamori S, Holt M, Stenius K, Lemke E, Gronborg M, Riedel D, Urlaub H, Schenck S, Brugger B, Ringler P, Muller S, Rammner B, Grater F, Hub J, De Groot B, Mieskes G, Moriyama Y, Klingauf J, Grubmuller H, Heuser J, Wieland F, Jahn R (2006) Molecular anatomy of a trafficking organelle. Cell 127 (4):831-846.
52. Breckenridge W C, Combos G, Morgan I G (1972) The lipid composition of adult rat brain synaptosomal plasma membranes. Biochim Biophys Acta 266 (3):695-707.
53. Skrivanek J A, Ledeen R W, Margolis R U, Margolis R K (1982) Gangliosides associated with microsomal subfractions of brain: comparison with synaptic plasma membranes. J Neurobiol 13 (2):95-106.
54. Marxen P, Erdmann G, Bigalke H (1991) The translocation of botulinum A neurotoxin by chromaffin cells is promoted in low ionic strength solution and is insensitive to trypsin. Toxicon 29 (2):181-189.
55. Mason J T, Xu L, Sheng Z-m, He J, O'Leary T J (2006) Liposome polymerase chain reaction assay for the sub-attomolar detection of cholera toxin and botulinum neurotoxin type A. Nat Protoc 1 (4):2003-2011.
56. Lee A G (2003) Lipid-protein interactions in biological membranes: a structural perspective. Biochim Biophys Acta 1612 (1):1-40.
57. Wimley W C, White S H (2000) Designing transmembrane alpha-helices that insert spontaneously. Biochemistry 39 (15):4432-4442.
58. Paternostre M, Roux M, Rigaud J (1988) Mechanisms of membrane protein insertion into liposomes during reconstitution procedures involving the use of detergents. 1. Solubilization of large unilamellar liposomes (prepared by reversephase evaporation) by triton X-100, octyl glucoside, and sodium cholate. Biochemistry 27 (8):2668-2677.
59. Fischer A, Mushrush D J, Lacy D B, Montal M (2008) Botulinum neurotoxin devoid of receptor binding domain translocates active protease. PLoS Pathog 4 (12):e1000245.
60. Fischer A, Nakai Y, Eubanks L M, Clancy C M, Tepp W H, Pellett S, Dickerson T J, Johnson E A, Janda K D A, Montal M (2009) Bimodal modulation of the botulinum neurotoxin protein-conducting channel. Proc Natl Acad Sci USA 106 (5):1330-1335.
61. Koriazova L K, Montal M (2003) Translocation of botulinum neurotoxin light chain protease through the heavy chain channel. Nat Struct Biol 10 (1):13-18.
62. Shi J, Li T, Hou X, Cai K, Bao S, Liu H, Gao X, Xiao L, Tu W, Wang Q, Yin J, Wang H (2010) Recombinant luminal domain of human synaptotagmin II in combination with gangliosides inhibits the toxicity of botulinum neurotoxins in mice. Microb Infect 12 (4):319-323.
63. Wróblewski H (1979) Amphilphilic nature of spiralin, the major protein of the Spiroplasma citri cell membrane. Journal of Bacteriology 140 (2):738-741.
64. Shi X, Garcia G, Nambiar M, Gordon R (2008) Un-nicked BoNT/B activity in human SHSY-5Y neuronal cells. J Cell Biochem 105 (1):129-135.
65. Shi X, Garcia G E, Neill R J, Gordon R K (2009) TCEP treatment reduces proteolytic activity of BoNT/B in human neuronal SHSY-5Y cells. J Cell Biochem 107 (5):1021-1030.
66. Christian T, Shine N, Eaton L, Crawford K Comparison of Activity of Botulinum Neurotoxin Type A Holotoxin and Light Chain Using SNAPtide FRET Substrates. In: 5th International Conference on Basic and Therapeutic Aspects of Botulinum and Tetanus Toxins, Denver, Jul. 23 2005.
67. Frisk M L, Lin G, Johnson E A, Beebe D J (2011) Synaptotagmin II peptide-bead conjugate for botulinum toxin enrichment and detection in microchannels. Biosens Bioelectron 26 (5):1929-1935.
68. Rasooly R, Do P M (2010) Clostridium botulinum Neurotoxin Type B Is Heat-Stable in Milk and Not Inactivated by Pasteurization. J Agric Food Chem 58 (23):12557-12561.
69. Wang H-H, Riding S, Lindo P, Singh B R (2010) Endopeptidase Activities of Botulinum Neurotoxin Type B Complex, Holotoxin, and Light Chain. Appl Environ Microbiol 76 (19):6658-6663.
70. Endrukaite E, Christian T, Shine N FRET Peptide Substrates for Botulinum Neurotoxins Types A, B, C, and E. In: 46th Annual Interagency Botulinum Research Coordinating Committee Meeting, Alexandria, USA, Oct. 1 2009.
71. Ketelsen A, Eriksson H (2008) Sensitive detection of hydrophobic antigens using a novel lipid-aggregate based ELISA. J Immunol Methods 339 (2):195-204.
72. Takara M, Ito A S (2005) General and specific solvent effects in optical spectra of ortho-aminobenzoic acid. J Fluoresc 15 (2):171-177.
73. White S H (1976) The lipid bilayer as a solvent for small hydrophobic molecules. Nature 262 (5567):421-422.
74. Kraus E, Femfert U (1976) Proteinase K from the mold Tritirachium album Limber. Specificity and mode of action. Hoppe-Seyler's Zeitschrift für Physiologische Chemie 357 (7):937-947.
75. Yowler, B C and Schengrund C-L (2004) Glycosphingolipids—Sweets for botulinum neurotoxin. Glycoconjugate Journal 21: 287-293 (2004).
76. Nishiki, T., Ogasawara, J., Kamata, Y., Kozaki S. (1993) Solubilization and characterization of the acceptor for Clostridium botulinum type B neurotoxin from rat brain synaptic membranes. Biochem Biophys Acta, 1158: 333-338.
77. Dong, M., Yeh, F., Tepp, W. H., Dean, C., Johnson, E. A., Jaez, R., Chapman, E. (2006) SV2 is the protein receptor for botulinum neurotoxin A. Science, 312: 592-596.
78. Dong, M., Liu, H., Tepp, W. H., Johson, E. A., Janz, R., Chapman, E. R. (2008) Gylocosylated SV2A and SV2B mediate the entry of botulinum neurotoxin E into neurons. Mol Biol Cell, 19: 5226-5237.
79. Rummel, A., Marhold, S., Bigalke, H., Binz, T. (2004) The HCC-domain of botulinum neurotoxins A and B exhibits a singular ganglioside binding site displaying serotype specific carbohydrate interaction. Mol Microbiol, 51: 631-643.
80. Rummel, A. Karnath, T., Henke, T., Bigalke, H., Binz T. (2004) Synaptotagmins I and II Act as Nerve Cell Receptors for Botulinum Neurotoxin G. JBC, 279 30865-30870.
81. Estes, D. J. and Mayer, M. (2005) Giant liposomes in physiological buffer using electroformation in a flow chamber; Biochimica Et Biophysica Acta-Biomembranes 1712(2): 152-160.
82. Mertins, O., Sebben, M., Pohlmann, A. R., da Silveira, N. P. (2005) Production of soybean phosphatidylcholine-chitosan nanovesicles by reverse phase evaporation: a step by step study, Chem. Phys Lipids 138: 29-37.
83. Poras H, Ouimet T, Orng S V, Fournie-Zaluski M C, Popoff M R, et al. (2009) Detection and quantification of botulinum neurotoxin type a by a novel rapid in vitro fluorimetric assay. Applied and environmental Microbiology 75: 4382-4390.
84. Fournie-Zaluski Pharmaleads, Marie-Claude (2005) Peptide substrate selectively recognisable by a botulinum toxin A, BoNT/A and the use thereof. US Patent Office.
85. Anne C, Turcaud S, Blommaert A G, Darchen F, Johnson E A, at al. (2005) Partial protection against Botulinum B neurotoxin-induced blocking of exocytosis by a potent inhibitor of its metallopeptidase activity. Chembiochem: a European journal of chemical biology 6: 1375-1380.
86. Anne C, Cornille F, Lenoir C, Rogues B P (2001) High-throughput fluorogenic assay for determination of botulinum type B neurotoxin protease activity. Analytical biochemistry 291: 253-261.
87. Rogues BdISedIRMI, Institut National, Anne C (2003) Peptide substrate identified by type bont/b botulinus toxin and use thereof for assaying and/or detecting said toxin or corresponding inhibitors. US Patent Office.
88. Schmidt J J, Stafford R G (2011) Enhanced substrates for the protease activity of serotype A botulinum neurotoxin. WO Patent WO/2011/047,241.
89. Shine N R, Crawford K R, Eaton L J A (2001) Substrate peptides and assays for detecting and measuring proteolytic activity of serotype A neurotoxin from Clostridium botulinum. US Patent Office.
90. Joshi S G (2012) Detection of biologically active botulinum neurotoxin—a in serum using high-throughput FRET-assay. Journal of pharmacological and toxicological methods 65: 8-12.
91. Henkel J S, Jacobson M, Tepp W, Pier C, Johnson E A, et al. (2009) Catalytic properties of botulinum neurotoxin subtypes A3 and A4. Biochemistry 48: 2522-2528.
92. Dong M, Liu H, Tepp W H, Johnson E A, Janz R, et al. (2008) Glycosylated SV2A and SV2B mediate the entry of botulinum neurotoxin E into neurons. Molecular biology of the cell 19: 5226-5237.
93. Dong M, Yeh F, Tepp W H, Dean C, Johnson E A, et al. (2006) SV2 is the protein receptor for botulinum neurotoxin A. Science 312: 592-596.

1. A method of determining presence, amount and/or activity of a clostridial neurotoxin in a sample, the method comprising or consisting of the following steps:
(a) bringing said sample into contact with a liposome, said liposome comprising
(aa) at least one receptor on its outer surface, said receptor being capable of binding said neurotoxin and comprising or consisting of
(i) a glycolipid and
(ii) a peptide or protein;
and
(ab) a substrate in its interior, said substrate
(i) being cleavable by the peptidase comprised in said neurotoxin and
(ii) generating a detectable signal upon cleavage, said detectable signal preferably being generated by (1) the donor of a FRET pair, said donor exhibiting increased fluorescence upon cleavage by said peptidase, (2) a luminescent compound formed upon said cleavage, or (3) an enzyme formed upon said cleavage;
and
(b) determining whether an increase in signal occurs as compared to the absence of said sample,
wherein such increase is indicative of the presence of said neurotoxin and/or the degree of such increase is indicative of the amount and/or activity of said neurotoxin in said sample.

2. The method of claim 1, wherein said liposome comprises or consists of the following constituents:
(a) (i) one or more liposome-forming lipids, preferably at least one phosphatidylcholine and cholesterol, said phosphatidylcholine preferably being selected from the group consisting of SPC, DOPC, and POPC;
(ii) optionally tocopherol;
(b) said at least one receptor, wherein said receptor preferably comprises or consists of
(i) a glycolipid, preferably selected from the tri-sialo ganglioside GT1b, the di-sialo ganglioside GD1b and the di-sialo ganglioside GD1a; and
(ii) a peptide or protein selected from (1) SV2C or a fragment thereof, wherein said fragment is capable of binding to said neurotoxin and preferably comprises or consists of the luminal domain 4 and at least one transmembrane domain; (2) synaptotagmin I or II or a fragment thereof, wherein said fragment is capable of binding to said neurotoxin and preferably comprises or consists of the N-terminal extracellular domain and the transmembrane portion of synaptotagmin I or II; (3) SV2A or a fragment thereof, wherein said fragment is capable of binding to said neurotoxin and preferably comprises or consists of the luminal domain 4; and (4) SV2B or a fragment thereof, wherein said fragment is capable of binding to said neurotoxin and preferably comprises or consists of the luminal domain 4; and
(c) said substrate; and
(d) an aqueous medium in the interior of said liposome.

3. The method of claim 1 or 2, wherein said bringing into contact is effected at a pH between 6 and 8, preferably between 7 and 7.4, more preferably at about 7.2.

4. The method of claim 3, wherein, after step (a) and prior to step (b), the pH is changed to a value between 4 and 6, preferably between 5 and 5.4, more preferably about 5.2.

5. The method of any one of claims 1 to 4, wherein said liposome comprises or consists of:
(i) DOPC and/or POPC;
(ii) cholesterol;
(iii) GT1b, GD1b and/or GDla;
(iv) (1) SV2C or said fragment thereof; (2) synaptotagmin I or II or said fragment thereof; (3) SV2A or said fragment thereof; and/or (4) SV2B or said fragment thereof;
(v) said substrate;
(vi) an aqueous medium in the interior of said liposome; and
(vii) optionally tocopherol.

6. Use of a liposome as defined in any one of the preceding claims for determining presence, amount and/or activity of a clostridial neurotoxin.

7. A liposome as defined in any one of the preceding claims.

8. The method of any one of claims 1 to 5, wherein said sample is known to comprise or suspected of comprising neutralising antibodies against said neurotoxin, wherein said sample, prior to subjecting it to said method, is combined with a known amount or activity of said neurotoxin, and wherein a decreased amount or activity of said neurotoxin as determined by said method in comparison to a control sample is indicative of the presence of said neutralising antibodies, wherein said control sample comprises said known amount or activity of said neurotoxin but is free of said neutralising antibodies.

9. The method of any one of claims 1 to 5, wherein said sample comprises a test compound and a known amount or activity of said neurotoxin, wherein a decreased or increased amount or activity of said neurotoxin as determined by said method in comparison to a control sample is indicative of the test compound being an inhibitor or activator, respectively, of said neurotoxin, wherein said control sample comprises said known amount or activity of said neurotoxin but is free of said test compound.

10. A method of preparing a liposome, said method comprising or consisting of the following steps:
(a) dissolving (i) liposome-forming lipid(s), preferably at least one phosphatidylcholine and cholesterol, said phosphatidylcholine preferably being selected from the group consisting of SPC, DOPC, and POPC; (ii) GT1b; and optionally (iii) tocopherol in a suitable organic solvent;
(b) evaporating said organic solvent;
(c) resuspending the residue of step (b) in an aqueous medium, said aqueous medium comprising
(ca) at least one receptor, said receptor being capable of binding a clostridial neurotoxin and comprising or consisting of a glycolipid and a peptide or protein,
wherein preferably
(i) said glycolipid is selected from the tri-sialo ganglioside GT1b, the di-sialo ganglioside GD1b and the di-sialo ganglioside GD1a; and
(ii) said peptide or protein is selected from
(1) SV2C or a fragment thereof, wherein said fragment is capable of binding to said neurotoxin and preferably comprises the luminal domain 4 and at least one transmembrane domain;
(2) synaptotagmin I or II or a fragment thereof, wherein said fragment is capable of binding to said neurotoxin and preferably comprises the N-terminal extracellular domain and the transmembrane portion of synaptotagmin I or II; and
(3) SV2A, SV2B or a fragment of SV2A or SV2B, wherein said fragment is capable of binding to said neurotoxin and preferably comprises or consists of the luminal domain 4;
and
(cb) a substrate
(i) being cleavable by the peptidase comprised in said neurotoxin and
(ii) generating a detectable signal upon cleavage, said detectable signal preferably being generated by (1) the donor of a FRET pair, said donor exhibiting increased fluorescence upon cleavage by said peptidase, (2) a luminescent compound formed upon said cleavage, or (3) an enzyme formed upon said cleavage;
(d) extruding the suspension obtained in step (c) through a suitable membrane; and
(e) optionally purifying the liposomes obtained in step (d), preferably by means of size exclusion chromatography.

11. The method of any one of claims 1 to 5 or 8 to 10, the use of claim 6, or the liposome of claim 7, wherein said substrate consists of or comprises a compound of the following formula (I):
X-L-Y;
wherein
L is a peptide or protein comprising or consisting of a sequence which is cleavable by said peptidase;
“—” denotes a covalent bond,
X-L-Y is preferably soluble in aqueous medium and/or free of any transmembrane domain or membrane anchor; and
(a) X is moiety comprising or consisting of a FRET donor or acceptor; and
Y is a moiety comprising or consisting of a FRET acceptor if X comprises or consists of a donor, or a FRET donor if X comprises or consists of an acceptor; or
(b) X is a fragment of an enzyme, said enzyme preferably being luciferase; and
Y is another fragment of said enzyme, said enzyme preferably being luciferase, wherein, upon cleavage of L by the peptidase of said neurotoxin, a functional enzyme comprising X and Y, said functional enzyme preferably having luciferase activity, is formed.

12. The method, use or liposome of claim 11, wherein L comprises or consists of
(i) SNAP-25 or a fragment thereof, said fragment being cleavable by the peptidase comprised in said neurotoxin and preferably being selected from a sequence comprising or consisting of residues 93 to 206, 146 to 203, or 156 to 184 of SNAP-25;
(ii) VAMP-2, VAMP-1, VAMP-3 or a fragment thereof, said fragment being cleavable by the peptidase comprised in said neurotoxin and preferably being selected from a sequence comprising or consisting of residues 30 to 62, 30 to 86, 38 to 62, 47 to 96, or 62 to 86 of VAMP-2; and/or
(iii) Syntaxin-1, Syntaxin-2, Syntaxin-3, or a fragment thereof, said fragment being cleavable by the peptidase comprised in said neurotoxin and preferably being a sequence comprising or consisting of residues 196 to 259 of Syntaxin-1.

13. The method, use or liposome of claim 11 or 12, wherein said substrate is selected from SNAPtide, SNAP Etide, VAMPtide, and SYNTAXide.

14. The method, use or liposome of any one of claims 11 to 13, wherein
(a) said neurotoxin is Botulinum neurotoxin type A; said receptor comprises or consists of (i) GT1b and (ii) SV2C or said fragment thereof; and L comprises or consists of SNAP-25 or said fragment thereof, said fragment preferably consisting of residues 146 to 203 of SNAP-25;
(b) said neurotoxin is Botulinum neurotoxin type B; said receptor comprises or consists of (i) GT1b and (ii) synaptotagmin II or I or said fragment thereof; and L comprises or consists of VAMP-2, VAMP-1, VAMP-3 or said fragment of any of these, said fragment preferably consisting of residues 62 to 86 of VAMP-2;
(c) said neurotoxin is Botulinum neurotoxin type C1; said receptor comprises or consists of GT1b; and L comprises or consists of SNAP-25, said fragment thereof, said fragment thereof preferably consisting of residues 93 to 206 of SNAP-25, Syntaxin-1, or said fragment thereof, said fragment preferably consisting of residues 196 to 259 of Syntaxin-1;
(d) said neurotoxin is Botulinum neurotoxin type D; said receptor comprises or consists of (i) GT1b and (ii) SV2A, SV2B, SV2C or the fragment of any of these as defined above; and L comprises or consists of VAMP-2, VAMP, VAMP-3 or said fragment of any of these, said fragment preferably consisting of residues 38 to 62 of VAMP-2;
(e) said neurotoxin is Botulinum neurotoxin type E; said receptor comprises or consists of (i) GT1b and (ii) SV2A, SV2B, or the fragment of any of these as defined above; and L comprises or consists of SNAP-25 or said fragment thereof, said fragment preferably consisting of residues 156 to 184 of SNAP-25;
(f) said neurotoxin is Botulinum neurotoxin type F; said receptor comprises or consists of (i) GT1b and (ii) SV2A, SV2B, SV2C, or the fragment of any of these as defined above, said fragment of SV2A and SV2B preferably comprising or consisting of the three luminal domains of SV2A and SV2B, respectively; and L comprises or consists of VAMP-2, VAMP, VAMP-3 or said fragment of any of these, said fragment preferably consisting of residues 30 to 62 of VAMP-2;
(g) said neurotoxin is Botulinum neurotoxin type G; said receptor comprises or consists of (i) GT1b and (ii) synaptotagmin I or II or said fragment thereof; and L comprises or consists of VAMP-2, VAMP, VAMP-3 or said fragment of any of these, said fragment preferably consisting of residues 47 to 96 of VAMP-2 and/or
(h) said neurotoxin is Tetanus neurotoxin; said receptor comprises or consists of (i) GT1b and (ii) SV2A, SV2B, SV2C, the fragment of any of these as defined above, or a GPI-anchored glycoprotein such as Thy-1; and L comprises or consists of VAMP-2, VAMP, VAMP-3 or said fragment of any of these, said fragment preferably consisting of residues 30 to 86 of VAMP-2.

15. A kit comprising or consisting of
(a) at least one receptor, said receptor being capable of binding a clostridial neurotoxin and comprising (1) a glycolipid and (2) a peptide or protein;
(b) a substrate which (1) is cleavable by the peptidase comprised in said neurotoxin and (2) comprises a FRET pair, the donor of said FRET pair exhibiting increased fluorescence upon cleavage by said peptidase; and
(c) optionally one or more liposome-forming lipids.


SEQUENCE LISTING (21 sequences)

SEQ ID NO: 1 - protein, 376 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSFPDVIKPLQSDEYALLTRNVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKTGCQITFDDDYSAYWIYFVNFLGTLAVLPGNIVSALL

SEQ ID NO: 2 - protein, 150 aa, Homo sapiens
FPDVIKPLQSDEYALLTRNVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKTGCQITFDDDYSAYWIYFVNFLGTLAVLPGNIVSALL

SEQ ID NO: 3 - protein, 307 aa, Artificial sequence
  /note=“Description of artificial sequence fusion protein”
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSVSASHPEALAAPVTTVATLVPHNATEPASPGEGKEDAFSKLKQKFMNELHKIPLPPWALIAIAIVAVLLVVTCCFCVCKKC

SEQ ID NO: 4 - protein, 82 aa, Rattus norvegicus
MVSASHPEALAAPVTTVATLVPHNATEPASPGEGKEDAFSKLKQKFMNELHKIPLPPWALIAIAIVAVLLVVTCCFCVCKKC

SEQ ID NO: 5 - protein, 48 aa, Rattus norvegicus
KEDAFSKLKQKFMNELHKIPLPPWALIAIAIVAVLLVVTCCFCVCKKC

SEQ ID NO: 6 - protein, 316 aa, Artificial sequence
  /note=“Description of artificial sequence fusion protein”
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMRNIFKRNQEPIVAPATTTATMPLAPAAPADNSTESTGTGESQEDMFAKLKDKFFNEINKIPLPPWALIAMAVVAGLLLLTCCFCICKKC

SEQ ID NO: 7 - protein, 90 aa, Rattus norvegicus
MRNIFKRNQEPIVAPATTTATMPLAPAAPADNSTESTGTGESQEDMFAKLKDKFFNEINKIPLPPWALIAMAVVAGLLLLTCCFCICKKC

SEQ ID NO: 8 - protein, 47 aa, Rattus norvegicus
EDMFAKLKDKFFNEINKIPLPPWALIAMAVVAGLLLLTCCFCICKKC

SEQ ID NO: 9 - protein, 377 aa, Artificial sequence
  /note=“Description of artificial sequence fusion protein”
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPDMIRHLQAVDYAARTKVFPGERVEHVTFNFTLENQIHRGGQYFNDKFIGLRLKSVSFEDSLFEECYFEDVTSSNTFFRNCTFINTVFYNTDLFEYKFVNSRLVNSTFLHNKEGCPLDVTGTGEGAYMVYFVSFLGTLAVLPGNIVSALLM

SEQ ID NO: 10 - protein, 151 aa, Rattus norvegicus
PDMIRHLQAVDYAARTKVFPGERVEHVTFNFTLENQIHRGGQYFNDKFIGLRLKSVSFEDSLFEECYFEDVTSSNTFFRNCTFINTVFYNTDLFEYKFVNSRLVNSTFLHNKEGCPLDVTGTGEGAYMVYFVSFLGTLAVLPGNIVSALLM

SEQ ID NO: 11 - protein, 374 aa, Artificial sequence
  /note=“Description of artificial sequence fusion protein”
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDMIRYFQDEEYKSKMKVFFGEHVHGATINFTMENQIHQHGKLVNDKFIKMYFKHVLFEDTFFDKCYFEDVTSTDTYFKNCTIESTTFYNTDLYKHKFIDCRFINSTFLEQKEGCHMDFEEDNDFLIYLVSFLGSLSVLPGNIISALLM

SEQ ID NO: 12 - protein, 148 aa, Rattus norvegicus
DMIRYFQDEEYKSKMKVFFGEHVHGATINFTMENQIHQHGKLVNDKFIKMYFKHVLFEDTFFDKCYFEDVTSTDTYFKNCTIESTTFYNTDLYKHKFIDCRFINSTFLEQKEGCHMDFEEDNDFLIYLVSFLGSLSVLPGNIISALLM

SEQ ID NO: 13 - protein, 206 aa, Rattus norvegicus
MAEDADMRNELEEMQRRADQLADESLESTRRMLQLVEESKDAGIRTLVMLDEQGEQLDRVEEGMNHINQDMKEAEKNLKDLGKCCGLYICPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 14 - protein, 67 aa, Homo sapiens
DARENEMDENLEQVSGIIGNLRHMALDMGNEIDTQNRQIDRIMEKADSNKTRIDEANQRATKMLGSG

SEQ ID NO: 15 - protein, 116 aa, Homo sapiens
MSATAATAPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 16 - protein, 116 aa, Rattus norvegicus
MSATAATVPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 17 - protein, 118 aa, Rattus norvegicus
MSAPAQPPAEGTEGAAPGGGPPGPPPNTTSNRRLQQTQAQVEEVVDIMRVNVDKVLERDQKLSELDDRADALQAGASVFESSAAKLKRKYWWKNCKMMIMLGAICAIIVVVIVIYIFT

SEQ ID NO: 18 - protein, 103 aa, Rattus norvegicus
MSTGVPSGSSAATGSNRRLQQTQNQVDEVVDIMRVNVDKVLERDQKLSELDDRADALQAGASQFETSAAKLKRKYWWKNCKMWAIGISVLVIIVIIIIVWCVS

SEQ ID NO: 19 - protein, 288 aa, Homo sapiens
MKDRTQELRTAKDSDDDDDVAVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 20 - protein, 288 aa, Rattus norvegicus
MKDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLENSIRELHDMFMDMAMLVESQGEMIDRIEYNVEHAVDYVERAVSDTKKAVKYQSKARRKKIMIIICCVILGIIIASTIGGIFG

SEQ ID NO: 21 - protein, 288 aa, Mus musculus
MKDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLETSIRELHDMFMDMAMLVESQGEMIDRIEYNVEHAVDYVERAVSDTKKAVKYQSKARRKKIMIIICCVILGIIIASTIGGIFG